SEQUENCE LISTING (2 sequences)

SEQ ID NO: 1 - protein, 9 aa, Artificial Sequence
  AH1 peptide epitope commercially available through Sigma
SPSYVYHQF

SEQ ID NO: 2 - protein, 19 aa, Artificial Sequence
  19-mer peptide containing AH1 epitope commercially available through Sigma
RVTYHSPSYVYHQFERRAK

(19) United States
(12) Patent Application Publication
(10) Pub. No.: US 2005/0221395 A1
(43) Pub. Date: Oct. 6, 2005

(54) Methods and products based on oligomerization of stress proteins

(76) Inventors: James R. Zabrecky, Waltham, MA (US); Chuanling Liu, Haverhill, MA (US); Stephen A. Monks, Arlington, MA (US); Andrew Wasserman, North Andover, MA (US); Pramod K. Srivastava, Avon, CT (US)

Correspondence Address: JONES DAY, 222 EAST 41ST ST, NEW YORK, NY, 10017, US

(21) Appl. No.: 10/506,097

(22) PCT Filed: Feb. 28, 2003
(86) PCT No.: PCT/US03/06298

§ 371 Date: Mar. 14, 2005

(30) Foreign Application Priority Data
Feb. 28, 2002 (US) 60361257

(51) Int. Cl. G01N 33/53; G01N 33/542; C12Q 1/42; A61K 31/19

(52) U.S. Cl. 435/7.9; 435/21; 514/183; 514/575

(57) ABSTRACT

In one aspect, the invention provides methods for determining the biological activity of heat shock proteins or heat shock protein-peptide complexes based on the ATPase activity or the multimeric structure of the heat shock proteins or heal shock protein-peptide complexes, and methods for screening agents that modulate the biological activity of heat shock proteins or heat shock protein-peptide complexes. In another aspect, the invention provides complexes, compositions and methods for enhancing the immunogenicity of a heat shock protein or a complex comprising a heal shock protein and an antigenic molecule.

### 1. INTRODUCTION

The present invention relates to the areas of immunology, immunotherapy of diseases, stress protein mediated immune modulation and vaccine development. More particularly, the present invention relates to methods for determining the biological activity of heat shock protein-peptide complexes and methods for screening agents that modulate the biological activity of heat shock protein-peptide complexes. The present invention also relates to methods for enhancing the immunogenicity of immunotherapeutic moieties, for example, heat shock protein-peptide complexes, by promoting their oligomerization.

### 2. BACKGROUND OF THE INVENTION

In modern medicine, immunotherapy or vaccination has virtually eradicated diseases such as polio, tetanus, tuberculosis, chicken pox, measles, hepatitis, etc. The approach using vaccinations has exploited the ability of the immune system to prevent infectious diseases. Vaccination with non-live materials such as proteins generally leads to an antibody response or CD4+ helper T-cell response. Raychaudhuri and Morrow (1993) Immunology Today 14:344-348. On the other hand, vaccination or infection with live materials such as live cells or infectious viruses generally leads to a CD8+ cytotoxic T-lymphocyte (CTL) response. A CTL response is crucial for protection against cancers, infectious viruses and certain bacteria. This poses a practical problem, for the only way to achieve a CTL response is to use live agents, which are themselves pathogenic. The problem is generally circumvented by using attenuated viral and bacterial strains or by killing whole cells which can be used for vaccination. These strategies have worked well but the use of attenuated strains always carries the risk that the attenuated agent may recombine genetically with host DNA and turn into a virulent strain. Thus, there is need for methods that can lead to CD8+ CTL response by vaccination with non-live materials such as proteins in a specific manner. It has been discovered that heat shock protein-peptide complexes have particular utility as vaccines against cancers and infectious diseases. (Srivastava et al., (1994) Curr. Op. Immu. 6:728; Srivastava (1993) Adv. Cancer Res. 62:153).

Heat shock proteins (hsps), also referred to as stress proteins, were first identified as proteins synthesized by cells in response to heat shock. Hsps have been classified into several families, based on their molecular weight, for example, hsp90, hsp70, hsp60, sm hsp etc. and each family consists of approximately 1-5 closely related proteins. Srivastava, 2002, Annu. Rev. Immunol. 20:395-425. Even though members within a family are closely related, there is little or no obvious homology among the individual hsp families. Heat shock proteins are expressed in all cells in all forms of life and in a variety of intracellular locations. They are expressed in vast quantities under normal conditions and their expression can be powerfully induced to much higher levels as a result of heat shock or other forms of stress, including exposure to toxins, oxidative stress, glucose deprivation etc. (see Srivastava, 2002, Annu. Rev. Immunol. 20:395-425).

Studies on the cellular response to heat shock and other physiological stresses revealed that the hsps are involved not only in cellular protection against these adverse conditions, but also in essential biochemical and immunological processes in unstressed cells. Hsps accomplish different kinds of chaperoning functions. For example, members of the hsp70 family located in the cell cytoplasm, nucleus, mitochondria, or endoplasmic reticulum (Lindquist et al., 1988, Ann. Rev. Genetics 22:631-677), are involved in the presentation of antigens to the cells of the immune system, and are also involved in the transfer, folding and assembly of proteins in normal cells. Hsps are also capable of binding proteins or peptides, and releasing the bound proteins or peptides in the presence of adenosine triphosphate (ATP) or low pH.

Srivastava et al. have demonstrated an immune response to methylcholanthrene-induced sarcomas of inbred mice (Srivastava et al., 1988, Immunol. Today 9:78-83). In these studies, it was found that the molecules responsible for the individually distinct immunogenicity of these tumors were glycoproteins of 96 kDa (gp96) and intracellular proteins of 84 to 86 kDa (Srivastava et al., 1986, Proc. Natl. Acad. Sci. USA 83:3407-3411; Ullrich et al., 1986, Proc. Natl. Acad. Sci. USA 83:3121-3125). Immunization of mice with gp96 or hsp84/86 isolated from a particular tumor rendered the mice immune to that particular tumor, but not to antigenically distinct tumors. Isolation and characterization of genes encoding gp96 and hsp84/86 revealed significant homology between them, and showed that gp96 and p84/86 were, respectively, the endoplasmic reticular and cytosolic counterparts of the same heat shock proteins (Srivastava et al, 1988, Immunogenetics 28:205-207; Srivastava et al., 1991, Curr. Top. Microbiol. Immunol. 167:109-123). Further, hsp70 was shown to elicit immunity to the tumor from which it was isolated but not to antigenically distinct tumors. However, hsp70 depleted of peptides was found to lose its immunogenic activity (Udono and Srivastava, 1993, J. Exp. Med. 178:1391-1396). These observations suggested that the heat shock proteins are not immunogenic per se, but form noncovalent complexes with antigenic peptides, and the complexes can elicit specific immunity to the antigenic peptides (Srivastava, 1993, Adv. Cancer Res. 62:153-177; Udono et al., 1994, J. Immunol., 152:5398-5403; Suto et al., 1995, Science 269:1585-1588).

This phenomenon has been observed in both tumor and viral models with known and unknown antigens (Srivastava et al. (1998) Immunity 8:657; Ciupitu et al. (1998) J. Exp. Med. 5: 685; Arnold et al. (1995) J. Exp. Med 182:885). The presence of an antigenic peptide bound to gp96, hsc70, and hsp84/hsp86 has been structurally demonstrated in cells for which the antigenic peptide is known (Nieland, et al. (1996) Proc. Nat'l. Acad. Sci. USA 93:6135; Breloer et al. (1998) Eur. J. Immunol. 28:1016; Ishii et al. (1999) J. Immunol. 162:1303). Vaccination with heat shock protein-peptide complexes is applicable for both the prophylactic (Srivastava et al. (1986) Proc. Nat'l. Acad. Sci. USA 83:3407; Ullrich et al. (1986) Proc. Nat'l. Acad. Sci. USA 83:3121; Peng et al. (1997) J. I. Meth. 204:13; Basu and Srivastava (1999) J. Exp. Med. 189:797) and therapeutic treatment of cancer (Tamura et al. (1997) Science 278:117; Yedavelli et al. (1999) Int. J. Mol. Med 3:243) and for the prevention of infectious diseases (Ciupitu et al. (1998) J. Exp. Med 5:685). The translation of this approach to immunotherapy of human cancer is currently under investigation using either gp96 complex (Janetzki, et al. (1998) J. Immunother. 4:269; Amato, et al. (1999) ASCO meeting, abstract 1278; Lewis, et al. (1999) ASCO meeting abstract 1687) or hsp70 complex as an autologous vaccine and the individual patient's cancers as a source of the heat shock proteins (Ménoret and Chandawarkar (1998) Semin. in Oncology 25:654).

Several hsps, including hsp 104, hsp90, hsp70 and hsp60 have been shown to possess ATPase activity (Scheibel, et al. (1997) J. Biol. Chem. 272:18608-18613; Richter, et al. (2001) J. Biol. Chem 276:33689; Lopez-Buesa, et al. (1998) Proc. Nat'l. Acad. Sci. 95:15253; Schirmer, et al. (1998) J. Biol. Chem. 273:15546). For hsp90, it has also been observed that the ATP binding and hydrolysis are associated with its molecular chaperone function (Obermann, et al. (1998) J. Cell Biol. 143:901; Panaretou, et al. (1998) EMBO J. 17:4829). However, the role of ATPase activity and conformational change in facilitating the in vivo biological activities of hsp90 remains the subject of further research.

Gp96, which is a member of the hsp90 family of heat shock proteins, has also been reported to possess ATPase activity (Li et al. (1993) EMBO J. 12:3143). However, this observation has been challenged by Wearsch and Nicchitta who proposed that the peptide binding activity of gp96 is adenine nucleotide independent, and that ATP binding and hydrolysis are not inherent properties of gp96 ((1997) J. Biol. Chem. 272:5152). These researchers also showed that the very mild ATPase activity in gp96 preparations was due to the minute amount of contaminating casein kinase II. Wearsch, Voglino and Nicchitta further reported that peptide binding to gp96 was identical in the presence or absence of ATP or ADP, and that the binding was stimulated 2-fold or 4-fold respectively following chemical denaturation/renaturation or transient heat shock ((1998) Biochem. 37:5709).

Gp96 has been shown to exist as a dimer of noncovalently associated subunits (Wearsch and Nicchitta (1996) Prot. Exp. Pur. 7:114). It has been proposed that peptides are assembled with higher order gp96 complexes (Sastry and Linderoth, J. Biol. Chem. 274:12023). It has further been shown that heat shock produces a tertiary conformational change which increases solvent and peptide accessibility to a hydrophobic domain (Wassenberg, et al. (2000) J. Biol. Chem. 275:22806). However, Wassenberg, et al. (supra) remarked that evidence in support of intrinsic ATP binding and ATPase activities is controversial and a consensus regarding the molecular basis of an adenosine nucleotide-mediated regulation of gp96-substrate interactions has yet to emerge. Despite the biophysical and biochemical studies of gp96, the regulation of interactions between gp96 and peptide substrates is still not fully understood. Even less is known about the effect of nucleotide binding and conformational changes on the functions of gp96-peptide complexes in the major histocompatibility complex class I antigen processing and presentation pathways.

As immunotherapeutic agents are being developed, it is becoming important to understand the mechanisms of these interactions and how they affect the biological activities of these complexes. In one aspect, the present invention provides methods for detecting and measuring the biological activities of hsp and hsp-antigen complexes which can be applied to determine the potency of immunotherapeutic agents based on hsp-antigen complexes.

Hsps such as gp96, hsp90, hsp70, calreticulin, hsp110 and grp170, are also known to chaperone a wide array of peptides (for review, see Srivastava et al., 1998, Immunity 8:657-665; Srivastava, 2002, Annu. Rev. Immunol. 20:395-425). For example, tumor-derived gp96 carries tumor antigenic peptide, gp96 preparations from virus-infected cells carry viral epitopes (Suto and Srivastava, 1995, Science 269:1585-1588), and gp96 preparations from cells transfected with model antigens such as ovalbumin or β-galactosidase are associated with the corresponding epitopes (Arnold et al., 1995, J. Exp. Med. 182:885-889; Breloer et al., 1998, Eur. J. Immunol. 28:1016-1021). Hsp-peptide complexes, whether isolated from cells (Tamura et al., 1997, Science 278:117-120), or reconstituted in vitro (Blachere et al., 1997, J. Exp. Med. 186:1183-1406) are excellent immunogens and have been used extensively to elicit CD8⁺ T-cell responses specific for the hsp-chaperoned antigenic peptides (for review, see Srivastava, 2002, Annu. Rev. Immunol. 20:395-425).

Non-covalent complexes of hsps and peptide purified from cancer cells, can be used for the treatment and prevention of cancer and have been described in PCT publications WO 96/10411, dated Apr. 11, 1996, and WO 97/10001, dated Mar. 20, 1997 (U.S. Pat. No. 5,750,119 issued Apr. 12, 1998, and U.S. Pat. No. 5,837,251 issued Nov. 17, 1998, respectively, each of which is incorporated by reference herein in its entirety). The isolation and purification of stress protein-antigen complexes has been described, for example, from pathogen-infected cells, and used for the treatment and prevention of infection caused by the pathogen, such as viruses, and other intracellular pathogens, including bacteria, protozoa, fungi and parasites (see, e.g., PCT Publication WO-95/24923, dated Sep. 21, 1995). Immunogenic stress protein-antigen complexes can also be prepared by in vitro complexing of stress protein and antigenic peptides, and the uses of such complexes for the treatment and prevention of cancer and infectious diseases has been described in PCT publication WO 97/10000, dated Mar. 20, 1997 (U.S. Pat. No. 6,030,618 issued Feb. 29, 2000). The use of stress protein-antigen complexes for sensitizing antigen presenting cells in vitro for use in adoptive immunotherapy is described in PCT publication WO 97/10002, dated Mar. 20, 1997 (see also U.S. Pat. No. 5,985,270 issued Nov. 16, 1999).

Complexes of hsps and peptides that elicit a greater immune response would be useful in order to enhance immunotherapy of infectious diseases as well as of cancer. In another aspect, the present invention provides complexes, compositions and methods for enhancing the immunogenicity of a heat shock protein or a complex comprising a heat shock protein and an antigenic molecule.

Citation or discussion of a reference herein shall not be construed as an admission that such is prior art to the present invention.

### 3. SUMMARY OF THE INVENTION

In one aspect, the present invention provides methods for detecting the biological activity of heat shock protein-peptide complexes. The invention also encompasses uses of the methods in determining the activity of immunotherapeutic agents comprising heat shock protein-peptide complexes; in assessing a subject's response to a vaccine or immunotherapy; and in the screening of agents that modulate the activity of heat shock protein-peptide complexes.

The hsp for use in the invention can be selected from any hsp family, including by way of example and not limitation, the hsp70 family, the hsp90 family and the hsp60 family.

Moreover, the hsp for use in the invention can be selected from any hsp, including but not limited to hsp90, gp96 (grp94), hsp104, hsp 70 and hsp 60. In a preferred embodiment, the hsp used in the invention is gp96.

Biological activity which can be assessed by the invention includes but is not limited to immunological activity, e.g., presentation of peptide antigen to antigen presenting cells (antigen re-presentation) and T-cell activation. In one embodiment, the term biological activity is limited to immunological activity. The immunological activities of hsp-peptide complexes are directly related to their usefulness in prophylactic and therapeutic applications.

Many other biological activities are known in the art, and are encompassed, such as but not limited to, induction of production of biological response modifiers, such as but not limited to cytokines, e.g., human macrophage chemo-attractant protein-1 (MCP-1), and nitric oxide (NO); binding of receptors such as CD91 (alpha-2-macroglobulin receptor, α2MR) and/or CD36; binding and release of an antigenic molecule; and the ability to cause the regression of tumors in animals, prolongation of survival of tumor-bearing animals and elimination of infection. The biological activities may occur or be observed in vivo and/or in vitro.

In one embodiment, the invention provides a method for detecting the biological activity of heat shock protein or heat shock protein-peptide complex comprising determining the ATPase activity of the heat shock protein or heat shock protein-peptide complex and using the ATPase activity of the heat shock protein or heat shock protein-peptide complex as an indicator of the biological activity. Any methods known in the art for determining ATPase activity can be used such as but not limited to enzyme reactions that involve ATP, such as but not limited to assays based on light-emitting enzymes, and methods that detect the concentrations and/or quantities of ATP, ADP, AMP and/or inorganic phosphate, such as but not limited to ion exchange chromatography, bioluminescence assay, HPLC, radioisotopic assays or an immunoaffinity assay.

In another embodiment, the invention provides a method for detecting the biological activity of heat shock protein or heat shock protein-peptide complex comprising determining the amounts of the oligomeric form of the heat shock protein or heat shock protein-peptide complex and using the presence of the oligomeric form of heat shock protein or heat shock protein-peptide complex as an indicator of the biological activity of the heat shock protein or heat shock protein-peptide complex. Any methods known in the art for determining the size and/or conformation of the heat shock protein or heat shock protein-peptide complex can be used such as but not limited to size exclusion chromatography, gel electrophoresis, immunoassay, gradient centrifugation, a filter, light scattering assay or analytical ultracentrifugation. In various embodiments, the dimeric form of heat shock protein or heat shock protein-peptide complex are preferred.

The methods of the invention can be used in combination to determine the biological activity of a composition comprising heat shock protein or heat shock protein-peptide complex.

In another embodiment, the invention provides a method for screening a potentially therapeutic compound that modulates the biological activity of a heat shock protein or heat shock protein-peptide complex. The method comprises the steps of measuring the ATPase activity of the heat shock protein or heat shock protein-peptide complex in the absence of a compound; contacting the heat shock protein or heat shock protein-peptide complex with a compound; comparing the ATPase activity of the heat shock protein or heat shock protein-peptide complex not contacted with the compound with the ATPase activity of the heat shock protein or heat shock protein-peptide complex contacted with the compound; and using any difference in the ATPase activity of the heat shock protein or heat shock protein-peptide complex contacted with the compound and the heat shock protein or heat shock protein-peptide complex not contacted with the compound as an indicator that the compound modulates the biological activity. The method can further comprise determining the ATPase activity in the presence of geldanamycin or any other specific inhibitors of nucleotide binding to hsps, such as NECA, wherein the ATPase activity that is inhibited by the presence of geldanamycin, or any other specific inhibitors of nucleotide binding, is used as an indicator of the biological activity. This step is particularly useful when other types of ATPase may be present.

In yet another embodiment, a method for screening a potentially therapeutic compound is provided that comprises measuring the amount of oligomeric form of the heat shock protein or heat shock protein-peptide complex in the absence of a compound; contacting the heat shock protein or heat shock protein-peptide complex with a compound; comparing the amount of oligomeric form of the heat shock protein or heat shock protein-peptide complex not contacted with the compound with the amount of oligomeric form of the heat shock protein or heat shock protein-peptide complex contacted with the compound; and using any difference in the amount of oligomeric form of the heat shock protein or heat shock protein-peptide complex contacted with the compound and the heat shock protein or heat shock protein-peptide complex not contacted with the compound as an indicator that the compound modulates the biological activity. In various embodiments, the dimeric form of heat shock protein or heat shock protein-peptide complex are preferred.

In yet another embodiment, the invention provides a method for screening variants of heat shock protein and complexes for their biological activity comprising determining the ATPase activity of the variants or the presence of oligomeric structures formed by the variants, or variants and their normal counterparts.

In yet another embodiment, the invention provides a method for modulating the biological activity of heat shock protein and complexes thereof comprising contacting the heat shock protein and complexes with compounds that modulate the ATPase activity or the oligomerization of the heat shock protein and complexes. Such compounds can be used to modulate the immune functions of a subject.

In yet another embodiment, the invention provides a method for diagnosing a condition in a subject that is due in part to either the abnormal or subnormal functioning of the subject's immune system, said method comprising using the ATPase activity of heat shock protein or heat shock protein-peptide complex or the presence of the oligomeric form of heat shock protein or heat shock protein-peptide complex obtained from the subject as an indicator of a biological activity of the heat shock protein or heat shock protein-peptide complex, wherein the biological activity is associated with one or more immune functions in the subject, whereby a change in ATPase activity or the amounts of oligomeric form indicates a change in the condition. In various embodiments, the dimeric form of heat shock protein or heat shock protein-peptide complex are preferred.

In a further embodiment, the invention provides a method for determining the prognosis of a cancer or an infectious disease in a subject comprising using the ATPase activity of heat shock protein or heat shock protein-peptide complex or the presence of the oligomeric form of heat shock protein or heat shock protein-peptide complex obtained from the subject as an indicator of a biological activity of the heat shock protein or heat shock protein-peptide complex, wherein the biological activity is associated with one or more immune functions that is responsive to or target the cells of the cancer or the agents that cause the infectious disease, whereby a change in ATPase activity or the amounts of oligomeric form indicates a change in the prognosis. In various embodiments, the dimeric form of heat shock protein or heat shock protein-peptide complex are preferred.

In one embodiment, the hsp or hsp-peptide complex can be obtained from a sample from a subject, such as a tissue sample or a blood sample; the hsp or hsp-peptide complex can then be further isolated and/or purified.

The methods of the invention can be used to provide a specific activity on mass basis of the heat shock protein or heat shock protein-peptide complex. The information can be used for controlling the quality or potency of heat shock protein or heat shock protein-peptide complex that is to be used in diagnostic or therapeutic applications. The information can be used to devise more economical and effective formulations and dosages.

The invention further provides a kit comprising a composition comprising heat shock protein or heat shock protein-peptide complex wherein the ATPase activity or the amount of the oligomeric form of the heat shock protein or heat shock protein-peptide complex is used as an indicator of the biological activity of the heat shock protein or heat shock protein-peptide complex, and instruction for determining the ATPase activity or the amount of the oligomeric form of the heat shock protein or heat shock protein-peptide complex. In various embodiments, the dimeric form of heat shock protein or heat shock protein-peptide complex are preferred.

In another aspect, the present invention provides complexes, compositions and methods for enhancing the immunogenicity of a heat shock protein or a complex comprising a heat shock protein and an antigenic molecule. The present invention also relates to a means of improving antigen delivery into cells thereby providing a more efficient and thus more potent vaccine preparation.

In one embodiment, the present invention provides a purified complex comprising an oligomerized, immunoactive heat shock protein and an antigenic molecule; wherein the heat shock protein has been oligomerized by contact with an oligomerizing agent, with the proviso that the oligomerizing agent is not 4,4′-dianilino-1,1′-binaphthyl-5,5′-disulfonic acid (“bis-ANS”), glutaraldehyde or sulfosuccinimidyl (4-azidosalicylamido) hexanoate (“SASD”). In another embodiment, the present invention provides a purified complex comprising an oligomerized, immunoactive heat shock protein and an antigenic molecule; wherein the heat shock protein has been oligomerized by covalent binding to an oligomerizing agent, with the proviso that the oligomerizing agent is not bis-ANS, glutaraldehyde or SASD. In certain embodiments, the heat shock protein is non-covalently bound to the oligomerizing agent. In some embodiments, the oligomerizing agent is selected from the group consisting of a bispecific or multivalent antibody that can bind a heat shock protein, biotin/avidin, biotin/streptavidin and derivatized poly ethylene glycol (“PEG”). In certain embodiments, the heat shock protein is gp96 or hsp90. In a preferred embodiment, the heat shock protein and antigenic molecule are isolated as a complex from a cell lysate, preferably from a cancerous cell or a cell infected with an agent displaying the antigenicity of an infectious disease agent.

In another embodiment, the present invention provides a population of purified oligomerized complexes, each complex in said population comprising an immunoactive heat shock protein and an antigenic molecule, wherein the complex has been oligomerized by contact with an oligomerizing agent, with the proviso that the oligomerizing agent is not bis-ANS, glutaraldehyde or SASD, and wherein at least one complex in said population comprises an antigenic molecule that is different from the antigenic molecule of another complex in said population. In another embodiment, the present invention provides a population of purified oligomerized complexes, each complex in said population comprising an immunoactive heat shock protein and an antigenic molecule, wherein the complex has been oligomerized by covalent binding to an oligomerizing agent, with the proviso that the oligomerizing agent is not bis-ANS, glutaraldehyde or SASD, and wherein at least one complex in said population comprises an antigenic molecule that is different from the antigenic molecule of another complex in said population. In some embodiments, the heat shock protein is non-covalently bound to the oligomerizing agent. In certain embodiments, the heat shock protein is gp96 or hsp90. In a preferred embodiment, the heat shock protein and antigenic molecule are isolated as a complex from a cell lysate, preferably from a cancerous cell or a cell infected with an agent displaying the antigenicity of an infectious disease agent.

In another embodiment, the present invention provides a purified complex comprising an oligomerized, immunoactive heat shock protein; wherein the heat shock protein has been oligomerized by contact with an oligomerizing agent, with the proviso that the oligomerizing agent is not bis-ANS, glutaraldehyde or SASD. In specific embodiments, the purified complex comprises an oligomerized, immunoactive heat shock protein; wherein the heat shock protein has been oligomerized by covalent binding to an oligomerizing agent, with the proviso that the oligomerizing agent is not bis-ANS, glutaraldehyde or SASD. Preferably, the complex further comprises an antigenic molecule and more preferably, the heat shock protein is in a complex with the antigenic molecule prior to contact with the oligomerizing agent. Alternatively, the heat shock protein is initially free of the antigenic molecule, but is contacted with the antigenic molecule after its oligomerization.

In another embodiment, the present invention provides a pharmaceutical composition comprising a pharmaceutically acceptable carrier and a purified complex, said complex comprising an oligomerized, immunoactive heat shock protein and an antigenic molecule, wherein the heat shock protein has been oligomerized by contact with an oligomerizing agent, with the proviso that the oligomerizing agent is not bis-ANS, glutaraldehyde or SASD. In some embodiments, the pharmaceutical composition comprises a pharmaceutically acceptable carrier and a purified complex, said complex comprising an oligomerized, immunoactive heat shock protein and an antigenic molecule, wherein the heat shock protein has been oligomerized by covalent binding to an oligomerizing agent, with the proviso that the oligomerizing agent is not bis-ANS, glutaraldehyde or SASD. In some embodiments, the pharmaceutical composition comprises a pharmaceutically acceptable carrier and therapeutically effective dose of a purified complex, said complex comprising an oligomerized, immunoactive heat shock protein and an antigenic molecule, wherein the heat shock protein has been oligomerized by covalent binding to an oligomerizing agent. In other embodiments, the pharmaceutical composition comprises a pharmaceutically acceptable carrier and a purified complex, said complex comprising an oligomerized, immunoactive heat shock protein, an antigenic molecule and a pharmaceutically acceptable carrier, wherein the heat shock protein has been oligomerized by covalent binding to an oligomerizing agent and wherein the pharmaceutical composition is present in a syringe. In specific embodiments, the heat shock protein is non-covalently bound to the oligomerizing agent. In certain embodiments, the heat shock protein is gp96 or hsp90. In a preferred embodiment, the heat shock protein and antigenic molecule are isolated as a complex from a cell lysate, preferably from a cancerous cell or a cell infected with an agent displaying the antigenicity of an infectious disease agent. In another preferred embodiment, the complex in the pharmaceutical composition is present in an amount effective for treatment or prevention of cancer or an infectious disease.

In yet another embodiment, the present invention provides a kit comprising a purified complex comprising an oligomerized, immunoactive heat shock protein; wherein the heat shock protein has been oligomerized by contact with an oligomerizing agent, with the proviso that the oligomerizing agent is not bis-ANS, glutaraldehyde or SASD. In certain embodiments, the kit comprises a purified complex comprising an oligomerized, immunoactive heat shock protein; wherein the heat shock protein has been oligomerized by covalent binding to an oligomerizing agent, with the proviso that the oligomerizing agent is not bis-ANS, glutaraldehyde or SASD. In some embodiments, the heat shock protein is non-covalently bound to the oligomerizing agent. In some embodiments, the heat shock protein is gp96 or hsp90. Preferably, the kit comprises a complex that further comprises an antigenic molecule. In a preferred embodiment, the heat shock protein and antigenic molecule are isolated as a complex from a cell lysate, preferably from a cancerous cell or a cell infected with an agent displaying the antigenicity of an infectious disease agent.

In another embodiment, the present invention provides a method of enhancing the antigenicity or immunogenicity of a complex comprising an immunoactive heat shock protein and an antigenic molecule by contacting the complex with an amount of an oligomerizing agent sufficient to cause oligomerization of the complex, with the proviso that the oligomerizing agent is not bis-ANS, glutaraldehyde or SASD. In another embodiment, a method of enhancing the antigenicity or immunogenicity of a complex comprises contacting the complex, said complex comprising an immunoactive heat shock protein and an antigenic molecule, with an amount of an oligomerizing agent sufficient to cause oligomerization of the complex, wherein the heat shock protein is covalently bound to the oligomerizing agent. In a specific embodiment, the complex used in the method comprises an immunoactive heat shock protein complexed with the antigenic molecule via a non-covalent bond. In a preferred embodiment, the antigenic molecule is a peptide. In another preferred embodiment, the complex comprising the immunoactive heat shock protein and the antigenic molecule are isolated from a cell lysate, preferably from a cancerous cell or a cell infected with an agent displaying the antigenicity of an infectious disease agent. In specific embodiments, the heat shock protein is gp96 or hsp90.

In another embodiment, the present invention provides a method of enhancing the antigenicity or immunogenicity of an immunoactive hsp-peptide complex by first contacting the hsp with an amount of an oligomerizing agent sufficient to cause oligomerization of the hsp, with the proviso that the oligomerizing agent is not bis-ANS, glutaraldehyde or SASD; and then contacting the oligomerized hsp with an antigenic molecule. In certain embodiments, the method comprises contacting the hsp with an amount of an oligomerizing agent sufficient to cause oligomerization of the hsp, wherein the heat shock protein is covalently bound to the oligomerizing agent, with the proviso that the oligomerizing agent is not bis-ANS, glutaraldehyde or SASD; and then contacting the oligomerized hsp with an antigenic molecule.

In another embodiment, the present invention provides a method of treating or preventing a cancer or an infectious disease comprising administering to a subject in need of such treating or preventing, a therapeutically effective amount of a purified complex, wherein the complex comprises an oligomerized, immunoactive heat shock protein and an antigenic molecule; wherein the heat shock protein has been oligomerized by contact with an oligomerizing agent, with the proviso that the oligomerizing agent is not bis-ANS, glutaraldehyde or SASD, and wherein the antigenic molecule displays the antigenicity of a tumor-specific or tumor-associated antigen of said cancer or of an agent of said infectious disease, respectively. In certain embodiments, the complex used in the method comprises a heat shock protein that has been oligomerized by covalent binding to an oligomerizing agent. In some embodiments, the complex used in the method comprises a heat shock protein that has been oligomerized by covalent binding to an oligomerizing agent, with the proviso that the oligomerizing agent is not glutaraldehyde. In other embodiments, the oligomerizing agent is not bis-ANS or SASD. In certain embodiments, the heat shock protein is gp96 or hsp90. In a preferred embodiment, the heat shock protein and antigenic molecule are isolated as a complex from a cell lysate, preferably from a cancerous cell or a cell infected with an agent displaying the antigenicity of an infectious disease agent. In another preferred embodiment, the cell is obtained from the subject.

In another embodiment, the present invention provides a method of treating or preventing a cancer or an infectious disease comprising administering to a subject in need of such treating or preventing, a therapeutically effective amount of a pharmaceutical composition comprising a pharmaceutically acceptable carrier and a purified complex, said complex comprising an oligomerized, immunoactive heat shock protein and an antigenic molecule, wherein the heat shock protein has been oligomerized by contact with an oligomerizing agent, with the proviso that the oligomerizing agent is not bis-ANS, glutaraldehyde or SASD, and wherein the antigenic molecule displays the antigenicity of a tumor-specific or tumor-associated antigen of said cancer or of an agent of said infectious disease, respectively. In some embodiments, the method comprises administering a therapeutically effective amount of a pharmaceutical composition comprising a pharmaceutically acceptable carrier and a purified complex, said complex comprising an oligomerized, immunoactive heat shock protein and an antigenic molecule, wherein the heat shock protein has been oligomerized by covalent binding to an oligomerizing agent. In some embodiments, the complex used in the method comprises a heat shock protein that has been oligomerized by covalent binding to an oligomerizing agent, with the proviso that the oligomerizing agent is not glutaraldehyde. In other embodiments, the oligomerizing agent is not bis-ANS or SASD. In a specific embodiment, the method comprises administering a therapeutically effective amount of a pharmaceutical composition comprising a pharmaceutically acceptable carrier and a purified complex, said complex comprising an oligomerized, immunoactive heat shock protein and an antigenic molecule, wherein (a) the heat shock protein has been oligomerized by covalent binding to an oligomerizing agent, (b) the pharmaceutical composition is present in a syringe and (c) the antigenic molecule displays the antigenicity of a tumor-specific or tumor-associated antigen of a cancer or of an agent of an infectious disease. In certain embodiments, the heat shock protein is gp96 or hsp90. In a preferred embodiment, the complex comprising the immunoactive heat shock protein and the antigenic molecule are isolated from a cell lysate, preferably from a cancerous cell or a cell infected with an agent displaying the antigenicity of an infectious disease agent. In another preferred embodiment, the cell is obtained from the subject.

In another embodiment, the present invention provides a method of making a pharmaceutical composition comprising (a) contacting a complex with an amount of an oligomerizing agent sufficient to cause oligomerization of the complex, wherein the complex comprises an immunoactive heat shock protein and an antigenic molecule, with the proviso that the oligomerizing agent is not bis-ANS, glutaraldehyde or SASD; and (b) combining the oligomerized complex with a pharmaceutically acceptable carrier. In some embodiments, the method comprises (a) contacting a complex with an amount of an oligomerizing agent sufficient to cause oligomerization of the complex, wherein the complex comprises an immunoactive heat shock protein and an antigenic molecule, and wherein the oligomerizing agent is covalently bound to the oligomerizing agent, with the proviso that the oligomerizing agent is not bis-ANS, glutaraldehyde or SASD; and (b) combining the oligomerized complex with a pharmaceutically acceptable carrier. In certain embodiments, the heat shock protein is gp96 or hsp90. In a preferred embodiment, the heat shock protein and antigenic molecule are isolated as a complex from a cell lysate, preferably from a cancerous cell or a cell infected with an agent displaying the antigenicity of an infectious disease agent.

### 4. BRIEF DESCRIPTION OF THE FIGURES

FIG. 1. An SEC tracing indicating that when hsp-peptide complex is heated, the protein rapidly forms higher molecular weight aggregates.

FIG. 2. Graph A indicates that there is a decrease in dimer level after heating gp96 at 50° C. and 60° C. for 30 minutes, with the rate of decrease being faster when heated at 60° C. Neither sample contained dimers at 30 minutes. Graph B shows the decrease in the NECA binding ability of gp96 when heated to 50° C. and 60° C. for 30 minutes, with the rate of decrease being faster when heated at 60° C. Neither sample of gp96 could bind NECA at 30 minutes. Graphs C and D demonstrate the rapid drop in the ability of gp96 complex to stimulate murine APC secretion of MCP-1 and NO when the gp96 complex is heated to 50° C. and 60° C. for 30 minutes. Graphs E and F demonstrate the effect of acidic pH on levels of dimer and NECA binding ability of gp96, respectively. Both dimer levels and NECA binding ability are markedly reduced at pH 3.0. Graphs G and H demonstrate the effect of acidic pH on gp96 complex's ability to stimulate the secretion of MCP-1 and NO by murine APCs, respectively. Both MCP-1 and NO secretion are markedly reduced at pH 3.0.

FIG. 3. An SEC tracing of gp96 complex prepared from human ovarian tumor cells, where fractions 8-10 correspond to the dimeric form of the hsp-peptide complex.

FIG. 4. SEC tracing of individual fractions of gp96 complex prepared from human ovarian tumor cells, where fractions 8-10 correspond to the dimeric form of the hsp-peptide complex.

FIG. 5. A silver stained SDS-PAGE gel of the individual fractions represented in FIGS. 3 and 4. Fractions 2-5 contain mostly higher molecular weight aggregates of gp96, while fractions 8-10 represent the dimer.

FIG. 6. ATPase activity of the fractions represented in FIGS. 3-5. Fractions 8-10, representing the dimer, had the highest ATPase activity and the fractions representing the aggregates (fractions 2-5) and other species (fractions 11-15) had significantly less ATPase activity.

FIG. 7. Hybrid antigen re-presentation assay demonstrates that human derived-gp96 complexed with mouse antigen AH1 19mer causes a dose-dependent increase in antigen re-presentation activity, represented by IFN-γ secretion, as compared to either gp96 or AH1 19mer alone. The top panel shows IFN-γ levels for samples applied to both APCs and CTLs. The second and third panels are controls for CTLs and APCs alone. Each data set contains media alone, unloaded gp96 and AH1 19mer as negative controls and AH1 9mer peptide, which can exchange directly onto surface MHC 1 molecules, as the positive control. gp96/AH1 19mer complex was applied at 10 μg/mL or diluted with unloaded gp96 at 1:3, 1:10 or 1:30. The results show a dose dependent IFN-γ response and no stimulation by unloaded gp96. Controls indicate that the activity is specific for AH1 19mer-loaded gp96. When the protein-peptide complex samples were heated at 60° C. for 10 min, conditions known to cause complete aggregation of the protein, the ability to stimulate T-cells was abolished.

FIG. 8. Results of experiment where recombinant gp96 were incubated at the indicated temperatures for 10 minutes and then analyzed for % dimer and ATPase activity. The figure shows a direct correlation between temperature induced aggregation and loss of ATPase activity.

### 5. DETAILED DESCRIPTION OF THE INVENTION

In one aspect, the invention described herein provides methods for detecting and measuring the biological activities of heat shock proteins and heat shock protein-antigenic molecule complexes or heat shock protein-peptide complexes (“hsp-peptide complexes” or “hsp complexes”). The methods of the invention can be used to determine the prognosis for subjects with a disease, and a subject's response to treatment. The methods can also be used to screen for therapeutic agents that modulate the biological activity of hsp-peptide complexes; and to screen complexes formed with variant hsp with increased or decreased biological activity.

The invention is based in part on studies conducted to understand the relationship between the structure of gp96-peptide complex and its functional properties. The inventors have demonstrated that dimeric gp96 has ATPase activity, is capable of re-presentation of antigen to specific T lymphocytes, and can induce MCP-1 and nitric oxide (NO) production. Higher molecular weight aggregates can be generated by heat treatment, as exemplified herein, and this form of aggregated gp96 is inactive in all four of the biological assays. Data provided in section 6 also demonstrate the loss of antigen re-presentation, MCP-1 production and NO production concomitant with loss of dimeric gp96.

The results of the inventors' studies prove that ATPase activity is an intrinsic function of gp96; ATPase activity is associated with the dimeric form of gp96; dimeric gp96 is necessary for ATPase activity; ATPase activity is not associated with forms of gp96, e.g., higher molecular weight aggregates of gp96, that are believed to be inactive due to denaturation or other conformational changes. Based on these results, the inventors determined that ATPase activity is a relevant and reliable measure of biological activity and stability based on the correlations of dimeric gp96 with ATPase activity and dimeric gp96 with antigen re-presentation and T-cell activation.

With increasing research being conducted on the efficacy of hsps and hsp-peptide complexes in treating and diagnosing a variety of diseases, there is a need to quickly and cheaply determine the biological activity of hsp-peptide complexes. The invention provides methods for detecting biological activity based on ATPase activity and/or the presence of hsp-peptide complex oligomers, that are quantitative and sensitive.

In one embodiment, the invention provides a method for assessing the biological activity of hsp or hsp-peptide complex which is based on determining the ATPase activity of the hsp or hsp-peptide complex. The presence of ATPase activity would indicate that the hsp or hsp-peptide complex is biologically active, and the lack of ATPase activity would indicate that the hsp or hsp-peptide complex is biologically inactive.

As used herein the term “biological activity” includes but is not limited to immunological activity, e.g., presentation of peptide antigen to antigen presenting cells (antigen re-presentation) and T-cell activation. In one embodiment, the term biological activity is limited to immunological activity. The immunological activities of hsp-peptide complexes are directly related to their usefulness in prophylactic and therapeutic applications. Many other biological activities are known in the art, and are encompassed, such as but not limited to, induction of production of biological response modifiers, such as but not limited to cytokines, e.g., human macrophage chemo-attractant protein-1 (MCP-1), and nitric oxide (NO); binding of receptors such as CD91 (alpha-2-macroglobulin receptor, α2MR) and/or CD36; binding and release of an antigenic molecule; and the ability to cause the regression of tumors in animals, prolongation of survival of tumor-bearing animals and elimination of infection. The biological activities may occur or be observed in vivo and/or in vitro.

The term “hsp” indicates a heat shock protein that is not non-covalently bound to an antigenic molecule. The term “hsp-peptide complexes” indicates a complex comprising an antigenic molecule non-covalently bound to a heat shock protein. Preferably, the antigenic molecule is an antigenic protein or peptide. Heat shock proteins, which are also referred to interchangeably as stress proteins, useful in the practice of the instant invention can be selected from among any cellular protein that satisfies the following criteria: (1) it is a protein whose intracellular concentration increases when a cell is exposed to a stressful stimulus; (2) it is capable of binding other proteins or peptides; and (3) it is capable of releasing the bound proteins or peptides in the presence of adenosine triphosphate (ATP) or low pH, e.g., 1, 2, 3, 4, 5 or 6; or it is a protein showing at least 35% homology with any cellular protein having all of the above properties. Preferably, the hsps and hsp-peptide complexes are human hsps and hsp-peptide complexes.

Hsps have been categorized into hsp families, including, but not limited to: hsp90, hsp 70 and hsp 60. In one embodiment, the hsp and hsp-peptide complexes encompass members of a single hsp family, such as hsp70, hsp90, and hsp60. Species that have been demonstrated to have ATPase activity include, but are not limited to hsp90, gp96 (grp94), hsp104, hsp 70 and hsp 60. In certain embodiments, gp96, also known as grp94, is the preferred hsp, and gp96-antigenic molecule complex is the preferred hsp-peptide complex of the invention.

Many methods are available for detecting and/or measuring the ATPase activity of hsp or hsp-peptide complexes. Typically, such methods measure ATPase activity by determining the amount of ATP hydrolyzed, which can be determined either by loss of ATP, gain of ADP, gain of AMP and/or gain in inorganic phosphate. Non-limited examples include bioluminescence, HPLC and the immunoaffinity stripping technique involving [γ-³²P]-labeled ATP and thin layer chromatography. Details of such methods are described in section 5.2.

In another embodiment, the invention provides a method for assessing the biological activity of hsp or hsp-peptide complex which is based on determining the presence and/or concentration of oligomeric hsp or hsp-peptide complex. In a preferred embodiment, the oligomeric structure is a dimeric structure as observed in the case of gp96. Thus, without limitation and for illustration purpose only, the term dimeric or dimerization is used hereinbelow. The presence of dimeric hsp or hsp-peptide complex in the sample would indicate that the hsp or hsp-peptide complex in the sample is biologically active, and the lack of dimeric hsp or dimeric hsp-peptide complex would indicate that the biological activity of the hsp or hsp-peptide complex is diminished. Distinguishing the dimeric form from higher order aggregates, monomers and degradation products can be carried out by determining the size and/or shape of the hsp or hsp-peptide complex. The invention provides methods for detection of the dimeric form of an hsp by size, including, but not limited to: general chromatographic techniques, including but not limited to size exclusion chromatography and ion exchange chromatography; general electrophoretic techniques, including but not limited to one and two dimensional electrophoresis and capillary electrophoresis; mass spectroscopy; light scattering assay; analytical ultracentrifugation (AUC); and the use of a filter, including but not limited to a molecular weight cutoff filter. Details of such methods are described in section 5.3.

The invention also provides methods for detection of the dimeric form of an hsp by conformation, including, but not limited to the use of polyclonal or monoclonal antibodies directed specifically against particular conformations of proteins. One embodiment includes the use of antibodies directed against the monomeric and oligomeric conformations of hsp. In a preferred embodiment, such antibodies do not bind forms of hsp or hsp-peptide complexes other than the dimeric form of the hsp and/or hsp-peptide complex. Details of such methods are described in section 5.10.

In a specific embodiment the method for assessing the biological activity of hsp or hsp-peptide complex encompasses both detecting and/or measuring the ATPase activity and determining the presence and/or concentration of dimeric hsp or dimeric hsp-peptide complex.

The methods of the invention have many areas of applications. In one instance, the methods can be used in the commercial production of hsp-peptide complexes. The invention provides a quick and inexpensive means of controlling and monitoring the quality of hsp-peptide complex with respect to its biological activity as an alternative to conducting an expensive and time consuming cell-based biological assays. Details of this method are described in section 5.5.

In yet another embodiment, the invention provides methods for providing a diagnosis of a disease with an immune component, or the prognosis of cancer or an infectious disease in a subject. Details of such methods are described in section 5.6.

The invention can also be used to screen for therapeutic agents that modulate the biological activities of hsp or hsp-peptide complexes. Details of such methods are described in section 5.8.

In yet another embodiment, the methods of the invention can be used to screen variants, fragments, and derivatives of hsps and complexes thereof, that possess comparable biological activities of unmodified hsp or hsp-peptide complex. Preferably, the biological activity of variants, fragments, and derivatives of hsps and complexes thereof are higher relative to the unmodified hsp or hsp-peptide complex.

In yet another embodiment, as described in section 5.7, the invention provides methods of modulating biological activities of hsp or hsp-peptide complex by contacting the hsp or hsp-peptide complex with a compound that inhibits or promotes the ATPase activity of hsp or hsp-peptide complex, and/or that disrupts/destabilizes or stabilizes the dimeric structure of hsp or hsp-peptide complex.

In another aspect, the present invention relates to using an oligomerizing agent to promote oligomerization of heat shock proteins or an immunoactive complex comprising heat shock proteins. In a preferred embodiment, the oligomerizing agent will promote the formation of dimeric species of gp96 or a multiplicity thereof. In particular, the invention provides formulations of a complex comprising immunologically active moieties, for example, heat shock proteins, oligomerized with an oligomerizing agent. Methods of use of the formulation for the prevention and treatment of cancer and infectious diseases, and for eliciting an immune response in a subject, are also provided. The invention is useful in various situations, such as where it is desirable to increase the biological potency of an immunotherapeutic moiety; to improve vaccine delivery into antigen presenting cells by specific and/or alternate receptor or non-receptor mediated events; to improve delivery of an immunotherapeutic moiety; to improve the adjuvant capabilities of an immunotherapeutic moiety; or to capture secondary immunotherapeutic/immunoactive moieties and delivering them into an antigen presenting cell via specific receptor-mediated uptake.

The present invention provides a composition of a complex comprising immunologically active moieties, for example, heat shock proteins, oligomerized with an oligomerizing agent. The complex can further comprise one or more antigenic molecules, preferably peptides, that display antigenicity of an antigen of a cancer or of an agent of an infectious disease. In a preferred embodiment, the oligomerized heat shock proteins and hsp-antigenic molecule complexes of the invention have ATPase activity. In a specific embodiment, the oligomerized heat shock proteins of the invention are not denatured, e.g., heat denatured hsps. As used herein, “oligomer” includes “dimer,” “trimer,” and higher numbers of units, including polymers. In specific embodiment, an “oligomer” of gp96 is a dimer or a multiplicity of dimers.

As used herein, with respect to a complex, the term “purified” refers to preparations of the complex that are at least 60% complex by weight of total protein. In one embodiment, it refers to at least 70% by weight of total protein. In another embodiment, it refers to at least 80% by weight of total protein. In another embodiment, it refers to at least 90% by weight of total protein. In another embodiment, it refers to at least 95% by weight of total protein and in another embodiment, it refers to at least 99% by weight of total protein. As used herein, with respect to a heat shock protein, the term “purified” refers to preparations of the hsp that are at least 60% hsp by weight of total protein. In one embodiment, it refers to at least 70% by weight of total protein. In another embodiment, it refers to at least 80% by weight of total protein. In another embodiment, it refers to at least 90% by weight of total protein. In another embodiment, it refers to at least 95% by weight of total protein and in another embodiment, it refers to at least 99% by weight of total protein. In some embodiments, purity refers to homogeneity as assayed by appearance on an SDS-PAGE gel. In preferred embodiments, the hsp is non-covalently bound to antigenic molecules, e.g., peptides.

The present invention also provides a pharmaceutical composition comprising an amount of a complex effective for treatment or prevention of an infectious disease or cancer, and a pharmaceutically acceptable carrier, wherein said complex comprises a heat shock protein oligomerized with an oligomerizing agent. The complex can further comprises one or more antigenic molecules, preferably peptides, that display antigenicity of an antigen of a type of cancer or of an antigen of an agent of an infectious disease.

As used herein, unless otherwise indicated, the term “immunoactive hsp” refers to the ability of a hsp to modulate, preferably stimulate or enhance, an immune response, preferably an immune response directed against an antigenic molecule to which the hsp is complexed.

Many oligomerizing agents are known in the art. The term “oligomerizing agent” refers to a compound that promotes the oligomerization of other molecules, for example, the oligomerization of heat shock proteins. An oligomerizing agent can bind covalently or non-covalently with the molecule, e.g., a heat shock protein. In one embodiment, the oligomerizing agent can bind covalently to two or more heat shock proteins and thereby cause oligomerization of the heat shock proteins. In another embodiment, the oligomerizing agent can bind non-covalently to two or more heat shock proteins and thereby cause oligomerization of the heat shock proteins. In another embodiment, the oligomerizing agent can bind covalently to one heat shock protein molecule and promote oligomerization by binding non-covalently to a similar or different oligomerizing agent on another heat shock protein molecule. In certain embodiments, the two bound heat shock proteins are identical. In preferred embodiments, the oligomerized heat shock proteins are bound non-covalently to antigenic molecules, e.g., peptides.

As used herein, unless otherwise indicated, the terms “molecule,” “complex,” “heat shock protein,” “stress protein,” “antigenic molecule” and “oligomerizing agent,” when used in singular, also encompasses a plurarity of the molecules, and may refer to a population of the referred molecules.

In some embodiments of the invention, the heat shock protein displays antigenicity of an antigen of a cancer or of an agent of an infectious disease. As used herein, the terms “antigenicity” and “immunogenicity” refer to the ability of a molecule to bind antibody or major histocompatibility complex (“MHC”) molecules and generate an immune response, respectively. As used herein, “a type of cancer” refers to the cell type of the tissue of origin, e.g., breast, lung, ovarian. In one embodiment, the antigenic molecule displays the antigenicity of an antigen of an infectious agent. In another embodiment, the antigenic molecule displays the antigenicity of an antigen overexpressed in a cancer cell relative to its expression in a noncancerous cell of said cell type. For example, the antigenic molecule can be a tumor specific antigen or a tumor-associated antigen. In one embodiment, a tumor-associated antigen is an antigen that is expressed at a higher level in a tumor cell relative to a normal cell; a tumor-specific antigen is an antigen that is expressed only in a tumor cell and not in a normal cell.

The present invention further provides a purified population of complexes in which each complex comprises a heat shock protein oligomerized with an oligomerizing agent. The complex can further comprise one or more antigenic molecules, e.g., peptides, that display antigenicity of an antigen of a type of cancer or of an antigen of an agent of an infectious disease. In one embodiment, the heat shock proteins include, but are not limited to, hsp70, hsp90, gp96, calreticulin, hsp110, grp170, or a combination thereof.

The present invention provides a method of making a complex which is immunogenic against a cancer or an agent of infectious disease, the method comprising contacting a heat shock protein or a hsp-antigenic molecule complex with an amount of an oligomerizing agent sufficient to promote oligomerization of the heat shock protein. In one embodiment, the oligomerized complex comprises a heat shock protein covalently bound to an oligomerizing agent. In another embodiment, antigenic molecule displays antigenicity of an antigen of a cancer or of an agent of an infectious disease. In another embodiment, the oligomerized complex comprises an immunoactive heat shock protein. The invention further provides a composition made by the methods described herein. In a specific embodiment, the oligomerizing agent is not a lectin.

In one embodiment, the antigenic molecules are peptides complexed to hsps in vivo, and the complexes can be isolated from cells. Alternatively, the complexes can be produced in vitro from purified preparations of hsp and antigenic molecules. In another embodiment, antigens of cancers or infectious agents can be obtained by purification from natural sources, by chemical synthesis, or recombinantly, and through in vitro procedures such as those described herein.

In one embodiment, the present invention provides a method of making an oligomerized complex, wherein the complex comprises an immunoactive hsp and an antigenic molecule, by contacting the complex with an amount of an oligomerizing agent sufficient to promote oligomerization of the complex. In another embodiment, the oligomerized complex is prepared by first oligomerizing the immunoactive hsp by contacting the hsp with an amount of an oligomerizing agent sufficient to promote oligomerization of the hsp; and contacting the oligomerized hsp with antigenic molecules of interest.

In another embodiment, the present invention provides a method of making an oligomerized complex, comprising isolating an immunoactive hsp-antigenic molecule complex from a cell; removing the endogenous antigenic molecule, e.g., a peptide, from the hsp-antigenic molecule complex; contacting the hsp with an exogenous antigenic molecule of interest, e.g., a different peptide, wherein the hsp now forms a complex with the exogenous antigenic molecule; and contacting the newly formed complex with an amount of an oligomerizing agent sufficient to promote oligomerization of the newly formed complex. In another embodiment, the present invention provides a method of making an oligomerized complex, comprising isolating an immunoactive hsp-antigenic molecule complex from a cell; removing the endogenous antigenic molecule, e.g., a peptide, from the hsp-antigenic molecule complex; contacting the hsp with an amount of an oligomerizing agent sufficient to promote oligomerization of the hsp; and contacting the oligomerized hsp with an exogenous antigenic molecule of interest, e.g., a different peptide, wherein the oligomerized hsp now forms a complex with the exogenous antigenic molecule.

The present invention further provides a method of enhancing the immunogenicity of a complex, wherein the complex comprises a heat shock protein, comprising binding an oligomerizing agent covalently or noncovalently to the complex. In one embodiment, the complex comprises a heat shock protein associated with an antigenic molecule.

The compositions and methods of the present invention can be used in various situations. For example, the composition of the invention can be used to elicit an immune response in an individual or a subject in whom the treatment or prevention of a cancer or an infectious disease is desired. The individual or subject in whom treatment or prevention of a cancer or an infectious diseases is desired is an animal, preferably a mammal, a non-human primate, and most preferably human. The term “animal” as used herein includes, but is not limited to, companion animals (e.g., cats and dogs), zoo animals, wild animals, including deer, foxes and racoons, farm animals, livestock and fowl, including horses, cattle, sheep, pigs, turkeys, ducks, chicken, and rodents.

According to the invention, an administration of oligomerized immunoactive complexes to a subject results in modulating an immune response, e.g., eliciting, stimulating, enhancing, and/or sustaining an immune response in the subject against antigenic peptides specific to an antigen source of interest. The oligomerized complex may be administered as a single dose, or multiple doses. The immunogenic dose may differ for different subjects and different therapeutic or prophylactic applications.

In one embodiment, the invention provides for a method of inducing an immune response by a sub-immunogenic amount of a vaccine composition, wherein the oligomerization facilitates the induction of an immune response by an amount of vaccine composition which is otherwise insufficient for inducing the immune response when used without oligomerization.

The present invention can also be used to increase biological activity of an immunotherapeutic moiety by contacting the moiety with an oligomerizing agent to promote the oligomerization of the immunotherapeutic moiety. As used herein, the term “immunotherapeutic moiety” refers to a molecule that is part of an immunotherapeutic complex. As used herein, the term “oligomerization” refers to the process by which a polymer or polymer intermediate is formed. In one embodiment, the immunotherapeutic moiety forms a dimer or a multiplicity of dimers. In another embodiment, the immunotherapeutic moiety forms higher-order species, i.e., an oligomer with more than two subunits.

The present invention can also be used to increase vaccine uptake into antigen presenting cells (APCs) by receptor mediated events. The present invention can also be used to increase vaccine uptake into antigen presenting cells by non-receptor mediated events. For example, heat shock protein-associated antigenic peptides can be taken up by antigen presenting cells by non-receptor mediated events including, but not limited to, pinocytosis, phagocytosis and non-specific interactions with cell surface components and subsequent insertion and/or translocation of hsp-peptide complexes across the cell membrane. Oligomerization of heat shock proteins can increase such an uptake.

The present invention can further be used to improve the adjuvant capabilities of an immunotherapeutic moiety. As used herein, the term “adjuvant capabilities” refers to the ability of a nonantigenic substance that, in combination with an antigen, enhances immune response by, e.g., inducing an inflammatory response, which leads to a local influx of immunoactive cells, such as antibody-forming cells or T lymphocytes. Immunotherapeutic moieties with adjuvant capabilities can be used therapeutically in the preparation of vaccines, because they increase the production of antibodies or T lymphocytes against small quantities of antigen and lengthen the period of antibody production or T-cell activation. Oligomerization of the immunotherapeutic moiety can increase their adjuvant capabilities. As used herein, the immunotherapeutic moiety refers to a stress protein, e.g., a heat shock protein. In one embodiment, the heat shock protein is hsp70, hsp90, gp96, calreticulin, hsp110, grp170 or combinations thereof.

The present invention can further be used to enhance antigenicity or immunogenicity of an immunotherapeutic moiety such as a heat shock protein by, for example, contacting it with an oligomerizing agent in sufficient amounts so as to promote its oligomerization. Enhancement of antigenicity or immunogenicity can be demonstrated in several ways as discussed in Section 5.15. In a preferred embodiment, an assay for demonstrating enhanced antigenicity or immunogenicity includes, but is not limited to, an antigen re-presentation assay, for example, as described in Section 8.

As used herein, “modulate” refers to an alteration in the properties, for example, antigenicity or immunogenicity, of an immunotherapeutic moiety. In one embodiment, it refers to an increase, enhancement or stimulation of antigenicity or immunogenicity. In another embodiment, it refers to a decrease or suppression of antigenicity or immunogenicity. A “modulator,” as used herein, therefore refers to a compound that “modulates” the properties of another.

The present invention can also be used to improve delivery of an immunotherapeutic moiety.

The present invention further provides a method of capturing secondary immunotherapeutic moieties and delivering said moieties to an antigen presenting cell via receptor mediated uptake in a subject, comprising administering to a subject a composition of a complex comprising a first protein and a second protein in the presence of an oligomerizing agent. The complex can further comprise one or more molecules, preferably peptides, that display antigenicity of an antigen of a cancer or of an agent of an infectious disease. In one embodiment, the second protein is different from the first protein. In another embodiment, the antigenic molecules associated with the first protein can be taken up by an antigen presenting cell, but those associated with the second protein can not normally be taken up by an antigen presenting cell. The oligomerization of the first protein to the second enables the antigenic molecules associated with the second protein to be taken up by the antigen presenting cell.

In one embodiment, the first and the second proteins are both heat shock proteins. In another embodiment, the first protein is a heat shock protein including, but not limited to, hsp70, hsp90, gp96, calreticulin, hsp110, grp170 or combinations thereof, but the second protein is not a heat shock protein. In another embodiment, the first protein is not a heat shock protein, but the second is. In one embodiment, the first protein is further associated with an antigenic molecule that displays the antigenicity of an antigen of a cancer or of an antigen of an agent of an infectious disease. In another embodiment, the second protein is further associated with an antigenic molecule that displays the antigenicity of an antigen of a cancer or of an antigen of an agent of an infectious disease.

The present invention further provides a method of treating or preventing a cancer or an infectious disease comprising administering to a subject a composition of the invention. In one embodiment, the composition comprises a complex comprising a heat shock protein, an antigenic molecule, and an oligomerizing agent, wherein the antigenic molecule displays the antigenicity of an antigen of said cancer or of an agent of said infectious disease.

In another embodiment, the composition is a pharmaceutical composition comprising the above-described complex and a pharmaceutically acceptable carrier. In another embodiment, the pharmaceutical composition comprising the above-described complex and a pharmaceutically acceptable carrier is present in a container, for example, a vial or a syringe.

In yet another embodiment, the method of treating or preventing a type of cancer or an infectious disease comprises administering to a subject (a) one or more complexes of a heat shock protein, an oligomerizing agent and a first antigenic molecule, wherein the first antigenic molecule displays the antigenicity of an antigen of a cancer or of an antigen of an agent of an infectious disease, and (b) before, concurrently, or after administration of the complex, administering to the subject a composition comprising antigen presenting cells sensitized in vitro with a sensitizing amount of a second complex of a heat shock protein associated with an oligomerizing agent and a second antigenic molecule, wherein said second antigenic molecule displays the antigenicity of a second antigen of said cancer or of an agent of said infectious disease. The APC can be selected from among those antigen presenting cells known in the art, including but not limited to macrophages, dendritic cells, B lymphocytes, and a combination thereof, and are preferably macrophages. In one embodiment, the first complex is the same as the second complex used to sensitize the APCs. In another embodiment, the first complex is different from the second complex used to sensitize the APCs. In a specific embodiment wherein the APCs and the compositions of the invention are administered concurrently, the APCs and composition of the invention can be present in the same composition (comprising APCs and the complexes) or different composition. Adoptive immunotherapy (using sensitized APCs) according to the invention allows activation of immune antigen presenting cells by incubation with oligomerized molecule complexes. The reactivity against the tumor or infectious agent in vitro can be measured prior to use of the cells in vivo. This in vitro boost followed by clonal selection and/or expansion, and patient administration constitutes a useful therapeutic and/or prophylactic strategy.

In one embodiment, the immunoactive moiety of the complex, e.g. a heat shock protein complexed to a peptide that displays antigenicity of an antigen of a cancer or of an agent of an infectious disease, is autologous to the subject; that is, it is isolated from the cells of the subject himself (e.g., prepared from tumor biopsies of the patient when the treatment of cancer is desired). Alternatively, the complex can be allogeneic to the subject to whom a composition of the molecular complex of the invention is administered. In one embodiment, the complex is prepared in vitro, e.g., from cultured cells that recombinantly express a heat shock protein.

Exogenous antigens and fragments and derivatives thereof for use in complexing with heat shock proteins to generate the specific complexes can be selected from among those known in the art, as well as those readily identified by standard immunoassays known in the art by the ability to bind antibody or MHC molecules or generate immune response. Specific complexes of heat shock proteins and antigenic molecules can be isolated from cancer or precancerous tissue of a patient, or from a cancer cell line, or can be produced in vitro (as is necessary in the embodiment in which an exogenous antigen is used as the antigenic molecule).

The present invention further provides kits comprising a pharmaceutical formulation or composition comprising a complex of the invention. The invention also provides kits comprising a container comprising an immunoactive heat shock protein or a complex thereof, and an oligomerizing agent. Optionally, instructions for formulating the oligomerized complexes according to the methods of the invention can be included in the kits.

In a specific embodiment, the present invention relates to methods and compositions for prevention and treatment of primary and metastatic neoplastic diseases.

The therapeutic regimens and pharmaceutical compositions of the invention can be used with additional immune response heat shock proteins, therapeutic agents, or biological response modifiers including, but not limited to, cytokines, chemotherapeutic agents, immunotherapeutics, anti-angiogenic agents, hormones, antibodies, polynucleotides, radiation and photodynamic therapeutic agents, antibiotics, anti-virals, anti-protozoal compounds and anti-fungal compounds. In another embodiment, the compositions of the invention are administered with radiotherapy or one or more chemotherapeutic agents for the treatment of cancer. In another embodiment, the compositions of the invention are administered with anti-bacterial, anti-viral or anti-fungal agents for the treatment of infectious diseases.

In addition to cancer therapy, the compositions of the invention can be utilized for the prevention of a variety of cancers, e.g., in subjects who are predisposed as a result of familial history or in subjects with an enhanced risk to cancer due to environmental factors.

Specific therapeutic regimens, pharmaceutical compositions, and kits are also provided by the invention.

5.1 Heat Shock Protein Preparations

Heat shock proteins, which are also referred to interchangeably as stress proteins, useful in the practice of the instant invention can be selected from among any cellular protein that satisfies the following criteria: (1) it is a protein whose intracellular concentration increases when a cell is exposed to a stressful stimulus; (2) it is capable of binding other proteins or peptides; and (3) it is capable of releasing the bound proteins or peptides in the presence of adenosine triphosphate (ATP) or low pH, e.g., 1, 2, 3, 4, 5 or 6; or it is a protein showing at least 35% homology with any cellular protein having all of the above properties.

Where hsp-peptide complexes are used in conjunction with administration of a non-vaccine treatment modality, preferably, the peptides are antigenic or relevant to the condition. In particular preferred embodiments, it is contemplated that the therapeutic outcome of a treatment modality administered to a subject with a particular type of cancer is improved by the administration of a hsp-peptide complex wherein the peptide displays the antigenicity of an antigen of that type of cancer.

In the present invention, an hsp preparation can include but not be limited to unbound hsp70, hsp90, gp96, calreticulin, hsp110 or grp170 or noncovalent or covalent complexes thereof complexed to a peptide.

In one embodiment, covalent or non-covalent complexes of hsp70, hsp90, gp96, calreticulin, hsp110 or grp170 with peptides can be prepared and purified postoperatively from tumor cells obtained from the cancer patient for use as specific complexes in the compositions of the invention.

In accordance with the methods described herein, immunogenic or antigenic peptides that are endogenously complexed to hsps or MHC antigens can be used as specific antigenic molecules. For example, such peptides can be prepared that stimulate cytotoxic T-cell responses against different tumor antigens (e.g., tyrosinase, gp100, melan-A, gp75, mucins, etc.) and viral proteins including, but not limited to, proteins of immunodeficiency virus. type I (HIV-I), human immunodeficiency virus type II (HV-II), hepatitis type A, hepatitis type B, hepatitis type C, influenza, Varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus and polio virus. In the embodiment wherein the antigenic molecules are peptides complexed to Hsps in vivo, the complexes can be isolated from cells, or alternatively, produced in vitro from purified preparations each of hsps and antigenic molecules. In some embodiments, the antigenic molecules are exogenous antigens and fragments and derivatives thereof.

In another embodiment, antigens of cancers (e.g., tumors) or infectious agents (e.g., viral antigen, bacterial antigens, etc.) can be obtained by purification from natural sources, by chemical synthesis, or recombinantly, and, through in vitro procedures such as that described below, complexed to hsps.

In an embodiment wherein the specific hsp-antigenic molecule complex to be used is a complex that is produced in vivo in cells, exemplary purification procedures such as those described below can be employed. Alternatively, in an embodiment wherein one wishes to use antigenic molecules by complexing to Hsps in vitro, Hsps can be purified for such use from the endogenous hsp-peptide complexes in the presence of ATP or low pH, e.g., 1, 2, 3, 4, 5 or 6. Hsps can also be chemically synthesized or recombinantly produced. The protocols described herein may be used to isolate specific Hsp-peptide complexes or the Hsps alone, from any eukaryotic cells, for example, tissues, isolated cells, or immortalized eukaryote cell lines infected with a preselected intracellular pathogen, tumor cells or tumor cell lines.

5.1.1 Sources of Hsp-Peptide Complexes

The source from which hsp-peptide complexes are recovered may be selected on the basis of the intended use of the resulting hsp-peptide complexes. Since hsp-peptide complexes are found in all cells, any tissue or cell sample can be used as a source. Hsp-peptide complexes can also be released from cells, by necrotic cell death, into the cells' surroundings; thus body fluids, secretions, culture supernatant, fermentation broth, and the like can be a source from which hsp-peptide complexes are recovered.

Hsp-peptide complexes can be recovered from cancerous or infected cells. Infected and cancerous cells can be prepared in vitro from noncancerous or uninfected cells (e.g., normal cells), as appropriate by methods known in the art. (See for example U.S. Pat. No. 6,017,540, which is incorporated by reference herein in its entirety.)

For applications relating to treatment and prevention of infectious diseases, the antigenic hsp-peptide complexes can be recovered from any infected cell, including; whole tissues, isolated cells, and immortalized cell lines infected or transformed with an intracellular pathogen. The antigenic hsp-peptide complexes can be recovered from cells infected with an infectious agent, and in particular with an intracellular pathogen. It has been demonstrated that a vaccine containing antigenic hsp-peptide complexes isolated from cells infected with an intracellular pathogen and then administered to a mammal can effectively stimulate cellular immune responses against cells infected with the same pathogen. Specifically, the immune response is mediated through the cytotoxic T-cell cascade which targets and destroys cells containing intracellular pathogens. Though not limited to intracellular pathogens, an intracellular pathogen as used herein encompases any viable organism, including, but not limited to, viruses, bacteria, fungi, protozoa and intracellular parasites, capable of existing within a mammalian cell and causing a disease in the mammal.

Hsp-peptide complexes can be recovered from cells infected with viruses including, but not limited to, hepatitis type A, hepatitis type B, hepatitis type C, influenza, varicella, adenovirus, HSV-I, HSV-II, rinderpest rhinovirus, echovirus, rotavirus, respiratory synctial virus, papilloma virus, papova virus, cytomegalovirus, echinovirus, arbovirus, huntavirus, coxsachie virus, mumps virus, measles virus, rubella virus, polio virus, HIV-I, and HIV-II. In addition, antigenic hsp-peptide complexes can also be collected from cells transfected with a viral gene.

Hsp-peptide complexes can be recovered from bacteria-infected cells including, but not limited to, cells infected with bacteria causing tuberculosis, gonorrhea, typhoid, meningitis, osteomyelitis, meningococcal septicemia, endometritis, conjunctivitis, peritonitis, pyelonephritis, pharyngitis, septic arghritis, cellulitis, epiglottitis, salpingitis, otitis media, shigella dysentery, gastroenteritis, etc. In preferred embodiments, the hsps or hsp-peptide complexes may also be recovered from cells infected with intracellular bacteria, including, but not limited to, Mycobacteria, Rickettsia, Mycoplasma, Neisseria and Legionella.

Hsp-peptide complexes can also be recovered from cells infected with intracellular protoza, including, but not limited to, Leishmania, Kokzidioa, and Trypanosoma. Furthermore, hsps or hsp-peptide complexes can be recovered from cells infected with intracellular parasites including, but not limited to, Chlamydia and Rickettsia. Hsp-peptide complexes can also be recovered from cell lines infected with bacteria.

Tissues, or cells isolated from a cancer, including cancer that has metastasized to multiple sites, can be used as a source of hsps or hsp-peptide complexes in the present method. For example, leukemic cells circulating in blood, lymph or other body fluids can also be used, solid tumor tissue (e.g., primary tissue from a biopsy) can be used.

Hsp-peptide complexes may be recovered from tumor cells, including, but not limited to, for example, tumors that are mesenchymal in origin (sarcomas) i.e., fibrosarcomas; myxosarcomas; liposarcomas; chondrosarcomas; osteogenic sarcomas; angiosarcomas; endotheliosarcomas; lymphangiosarcomas; synoviosarcomas; mesotheliosarcomas; Ewing's tumors; myelogenous leukemias; monocytic leukemias; malignant lymphomas; lymphocytic leukemias; plasmacytomas; leiomyosarcomas and rhabdomyosarcoma. In addition, it is contemplated that this method can be used in the recovery of hsps or hsp-peptide complexes from tumor cells from tumors that are epithelial in origin (carcinomas) i.e., squamous cell or epidermal carcinomas; basal cell carcinomas; sweat gland carcinomas; sebaceous gland carcinomas; adenocarcinomas; papillary carcinomas; papillary adenocarcinomas; cystadenocarcinomas; medullary carcinomas; undifferentiated carcinomas (simplex carcinomas); bronchogenic carcinomas; bronchial carcinomas; melanocarcinomas; renal cell carcinomas; hepatocellular carcinomas; bile duct carcinomas; papillary carcinomas; transitional cell carcinomas; squamous cell carcinomas; choriocarcinomas; seminomas; embryonal carcinomas malignant teratomas and teratocarcinomas. Hsps or hsp-peptide complexes can also be recovered from cells of leukemia, e.g., acute lymphocytic leukemia and acute myelocytic leukemia (myeloblastic, promyelocytic, myelomonocytic, inonocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. hsp-peptide complexes may be recovered from tumor cells from tumors induced by chemical carcinogens or radiation. Chemical carcinogens include carcinogens associated with cigarette smoking, such as hydrocarbons and carcinogenic air, food, cosmetics or other pollutants. hsp-peptide complexes may be recovered from tumor cell lines.

For applications relating to diagnosis, prognosis of a disease, or to gauging the response to a treatment, particularly with immunotherapeutics or vacccines, hsp-peptide complexes may be recovered from body fluids, blood, lymph, etc.

5.1.2 Preparation and Purification of Hsp70-Peptide Complexes

The purification of hsp70-peptide complexes has been described previously, see, for example, Udono et al., 1993, J. Exp. Med. 178:1391-1396. A procedure that may be used, presented by way of example but not limitation, is as follows:

Initially, tumor cells are suspended in 3 volumes of 1× Lysis buffer consisting of 30 mM sodium bicarbonate pH 7.5, and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose equilibrated with phosphate buffered saline (PBS) containing 2 mM Ca²⁺ and 2 mM Mg²⁺. When the cells are lysed by mechanical shearing the supernatant is diluted with an equal volume of 2× lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against 10 mM Tris-Acetate pH 7.5, 0.1 mM EDTA, 10 nM NaCl, 1 mM PMSF. Then the dialyzate is centrifuged at 17,000 rpm (Sorvall SS34 rotor) for 20 minutes. Then the resulting supernatant is harvested and applied to a Mono Q FPLC column equilibrated in 20 mM Tris-Acetate pH 7.5, 20 mM NaCl, 0.1 mM EDTA and 15 mM 2-mercaptoethanol. The column is then developed with a 20 mM to 500 nM NaCl gradient and then eluted fractions fractionated by sodium dodecyl sulfate-polyacrylamide gel electrophoresis (SDS-PAGE) and characterized by immunoblotting using an appropriate anti-hsp70 antibody (such as from clone N27F3-4, from StressGen).

Fractions strongly immunoreactive with the anti-hsp70 antibody are pooled and the hsp70-peptide complexes precipitated with ammonium sulfate; specifically with a 50%-70% ammonium sulfate cut. The resulting precipitate is then harvested by centrifugation at 17,000 rpm (SS34 Sorvall rotor) and washed with 70% ammonium sulfate. The washed precipitate is then solubilized and any residual ammonium sulfate removed by gel filtration on a Sephadex® G25 column (Pharmacia). If necessary the hsp70 preparation thus obtained can be repurified through the Mono Q FPLC Column as described above.

The hsp70-peptide complex can be purified to apparent homogeneity using this method. Typically 1 mg of hsp70-peptide complex can be purified from 1 g of cells/tissue.

An improved method for purification of hsp70-peptide complexes comprises contacting cellular proteins with ADP or a nonhydrolyzable analog of ATP affixed to a solid substrate, such that hsp70 in the lysate can bind to the ADP or nonhydrolyzable ATP analog, and eluting the bound hsp70. A preferred method uses column chromatography with ADP affixed to a solid substratum (e.g., ADP-agarose). See, e.g., Peng, 1997, J. Immuno. Meth. 204:13-21. The resulting hsp70 preparations are higher in purity. The hsp70 yields are also increased significantly by about more than 10 fold. Alternatively, chromatography with nonhydrolyzable analogs of ATP, instead of ADP, can be used for purification of hsp70-peptide complexes. By way of example but not limitation, purification of hsp70-peptide complexes by ADP-agarose chromatography can be carried out as follows:

Meth A sarcoma cells (500 million cells) are homogenized in hypotonic buffer and the lysate is centrifuged at 100,000 g for 90 minutes at 4° C. The supernatant is applied to an ADP-agarose column. The column is washed in buffer and is eluted with S column volumes of 3 mM ADP. The hsp70-peptide complexes elute in fractions 2 through 10 of the total 15 fractions which elute. The eluted fractions are analyzed by SDS-PAGE. The hsp70-peptide complexes can be purified to apparent homogeneity using this procedure.

5.1.3 Preparation and Purification of Hsp90-Peptide Complexes

A procedure that can be used, presented by way of example and not limitation, is as follows:

Initially, tumor cells are suspended in 3 volumes of 1× Lysis buffer consisting of 30 mM sodium bicarbonate pH 7.5, and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate pH 7.5, 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose equilibrated with PBS containing 2 mM Ca²⁺ and 2 mM Mg²⁺. When the cells are lysed by mechanical shearing the supernatant is diluted with an equal volume of 2× Lysis buffer prior to mixing with Con A Sepharose. The supernatant is then allowed to bind to the Con A Sepharose for 2-3 hours at 4° C. The material that fails to bind is harvested and dialyzed for 36 hours (three times, 100 volumes each time) against pH 7.4, 1.0 mM EDTA, 250 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC column equilibrated with dialysis buffer. The proteins are then eluted with a salt gradient of 200 mM to 600 mM NaCl.

The eluted fractions are fractionated by SDS-PAGE and fractions containing the Hsp90-peptide complexes identified by immunoblotting using an anti-hsp90 antibody such as 3 G3 (Affinity Bioreagents). Hsp90-peptide complexes can be purified to apparent homogeneity using this procedure. Typically, 150-200 μg of Hsp90-peptide complex can be purified from 1 g of cells/tissue.

5.1.4 Preparation and Purification of Gp96-Peptide Complexes

A procedure that can be used, presented by way of example and not limitation, is as follows:

A pellet of tumors is resuspended in 3 volumes of buffer consisting of 30 mM sodium bicarbonate buffer (pH 7.5) and 1 mM PMSF and the cells allowed to swell on ice 20 minutes. The cell pellet is then homogenized in a Dounce homogenizer (the appropriate clearance of the homogenizer will vary according to each cell type) on ice until >95% cells are lysed.

The lysate is centrifuged at 1,000 g for 10 minutes to remove unbroken cells, nuclei and other debris. The supernatant from this centrifugation step is then recentrifuged at 100,000 g for 90 minutes. The gp96-peptide complex can be purified either from the 100,000 pellet or from the supernatant.

When purified from the supernatant, the supernatant is diluted with an equal volume of 2× lysis buffer and the supernatant mixed for 2-3 hours at 4° C. with Con A Sepharose equilibrated with PBS containing 2 mM Ca²⁺ and 2 mM Mg²⁺. Then, the slurry is packed into a column and washed with 1× lysis buffer until the OD₂₈₀ drops to baseline. Then, the column is washed with ⅓ column bed volume of 10% α-methyl mannoside (α-MM) dissolved in PBS containing 2 mM Ca²⁺ and 2 mM Mg²⁺, the column sealed with a piece of parafilm, and incubated at 37° C. for 15 minutes. Then the column is cooled to room temperature and the parafilm removed from the bottom of the column. Five column volumes of the α-MM buffer are applied to the column and the eluate analyzed by SDS-PAGE. Typically the resulting material is about 60-95% pure, however this depends upon the cell type and the tissue-to-lysis buffer ratio used. Then the sample is applied to a Mono Q FPLC column (Pharmacia) equilibrated with a buffer containing 5 mM sodium phosphate, pH 7. The proteins are then eluted from the column with a 0-1M NaCl gradient and the gp96 fraction elutes between 400 mM and 550 mM NaCl.

The procedure, however, may be modified by two additional steps, used either alone or in combination, to consistently produce apparently homogeneous gp96-peptide complexes. One optional step involves an ammonium sulfate precipitation prior to the Con A purification step and the other optional step involves DEAE-Sepharose purification after the Con A purification step in lieu of the Mono Q FPLC step.

In the first optional step, described by way of example as follows, the supernatant resulting from the 100,000 g centrifugation step is brought to a final concentration of 50% ammonium sulfate by the addition of ammonium sulfate. The ammonium sulfate is added slowly while gently stirring the solution in a beaker placed in a tray of ice water. The solution is stirred from about ½ to 12 hours at 4° C. and the resulting solution centrifuged at 6,000 rpm (Sorvall SS34 rotor). The supernatant resulting from this step is removed, brought to 70% ammonium sulfate saturation by the addition of ammonium sulfate solution, and centrifuged at 6,000 rpm (Sorvall SS34 rotor). The resulting pellet from this step is harvested and suspended in PBS containing 70% ammonium sulfate in order to rinse the pellet. This mixture is centrifuged at 6,000 rpm (Sorvall SS34 rotor) and the pellet dissolved in PBS containing 2 mM Ca²⁺ and Mg²⁺. Undissolved material is removed by a brief centrifugation at 15,000 rpm (Sorvall SS34 rotor). Then, the solution is mixed with Con A Sepharose and the procedure followed as before.

In the second optional step, described by way of example as follows, the gp96 containing fractions eluted from the Con A column are pooled and the buffer exchanged for 5 mM sodium phosphate buffer, pH 7, 300 mM NaCl by dialysis, or preferably by buffer exchange on a Sephadex G25 column. After buffer exchange, the solution is mixed with DEAE-Sepharose previously equilibrated with 5 mM sodium phosphate buffer, pH 7, 300 mM NaCl. The protein solution and the beads are mixed gently for 1 hour and poured into a column. Then, the column is washed with 5 mM sodium phosphate buffer, pH 7, 300 mM NaCl, until the absorbance at 280 nm drops to baseline. Then, the bound protein is eluted from the column with five volumes of 5 mM sodium phosphate buffer, pH 7, 700 mM NaCl. Protein containing fractions are pooled and diluted with 5 mM sodium phosphate buffer, pH 7 in order to lower the salt concentration to 175 mM. The resulting material then is applied to the Mono Q FPLC column (Pharmacia) equilibrated with 5 mM sodium phosphate buffer, pH 7 and the protein that binds to the Mono Q FPLC column (Pharmacia) is eluted as described before.

It is appreciated, however, that one skilled in the art may assess, by routine experimentation, the benefit of incorporating the second optional step into the purification protocol. In addition, it is appreciated also that the benefit of adding each of the optional steps will depend upon the source of the starting material.

When the gp96 fraction is isolated from the 100,000 g pellet, the pellet is suspended in 5 volumes of PBS containing either 1% sodium deoxycholate or 1% oxtyl glucopyranoside (but without the Mg²⁺ and Ca²⁺) and incubated on ice for 1 hour. The suspension is centrifuged at 20,000 g for 30 minutes and the resulting supernatant dialyzed against several changes of PBS (also without the M²⁺ and Ca²⁺) to remove the detergent. The dialysate is centrifuged at 100,000 g for 90 minutes, the supernatant harvested, and calcium and magnesium are added to the supernatant to give final concentrations of 2 mM, respectively. Then the sample is purified by either the unmodified or the modified method for isolating gp96-peptide complex from the 100,000 g supernatant, see above.

The gp96-peptide complexes can be purified to apparent homogeneity using this procedure. About 10-20 μg of gp96 can be isolated from 1 g cells/tissue.

Separation of the hsp from an gp96-peptide complex can be performed in the presence of ATP or low pH. These two methods may be used to elute the peptide from an gp96-peptide complex. The first approach involves incubating an gp96-peptide complex preparation in the presence of ATP. The other approach involves incubating an gp96-peptide complex preparation in a low pH buffer. These methods and any others known in the art may be applied to separate the hsp and peptide from an hsp-peptide complex.

5.1.5 Preparation and Purification of Hsp110-Peptide Complexes

A procedure, described by Wang et al., 2001, J. Immunol. 166 (1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCl, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose unbound material is first dialyzed against a solution of 20 mM Tris-HCl, pH 7.5; 100 mM NaCl; and 15 mM 2-ME, and then applied to a DEAE-Sepharose column and eluted by salt gradient from 100 to 500 mM NaCl. Fractions containing hsp110 are collected, dialyzed, and loaded onto a Mono Q (Pharmacia) 10/10 column equilibrated with 20 mM Tris-HCl, pH 7.5; 200 mM NaCl; and 15 mM 2-ME. The bound proteins are eluted with a 200-500 mM NaCl gradient. Fractions are analyzed by SDS-PAGE followed by immunoblotting with an Ab for hsp 110, as described by Wang et al., 1999, J. Immunol. 162:3378. Pooled fractions containing hsp110 are concentrated by Centriplus (Amicon, Beverly, Mass.) and applied to a Superose 12 column (Pharmacia). Proteins are eluted by 40 mM Tris-HCl, pH 8.0; 150 mM NaCl; and 15 mM 2-ME with a flow rate of 0.2 ml/min.

5.1.6 Preparation and Purification of Grp-170-Peptide Complexes

A procedure, described by Wang et al., 2001, J. Immunol. 166 (1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCl, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose-bound material is first dialyzed against 20 mM Tris-HCl, pH 7.5, and 150 mM NaCl and then applied to a Mono Q column and eluted by a 150 to 400 mM NaCl gradient. Pooled fractions are concentrated and applied on the Superose 12 column (Pharmacia). Fractions containing homogeneous grp170 are collected.

5.1.7 Recombinant Expression of HSPs

Methods known in the art can be utilized to recombinantly produce hsps. A nucleic acid sequence encoding a heat shock protein can be inserted into an expression vector for propagation and expression in host cells.

An expression construct, as used herein, refers to a nucleotide sequence encoding an hsp operably associated with one or more regulatory regions which enables expression of the hsp in an appropriate host cell. “Operably-associated” refers to an association in which the regulatory regions and the hsp sequence to be expressed are joined and positioned in such a way as to permit transcription, and ultimately, translation.

The regulatory regions necessary for transcription of the hsp can be provided by the expression vector. A translation initiation codon (ATG) may also be provided if the hsp gene sequence lacking its cognate initiation codon is to be expressed. In a compatible host-construct system, cellular transcriptional factors, such as RNA polymerase, will bind to the regulatory regions on the expression construct to effect transcription of the modified hsp sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to the hsp gene sequence or to insert the hsp gene sequence into the cloning site of a vector, linkers or adapters providing the appropriate compatible restriction sites may be ligated to the ends of the cDNAs by techniques well known in the art (Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising an hsp sequence operably associated with regulatory regions can be directly introduced into appropriate host cells for expression and production of hsp-peptide complexes without further cloning. See, for example, U.S. Pat. No. 5,580,859. The expression constructs can also contain DNA sequences that facilitate integration of the hsp sequence into the genome of the host cell, e.g., via homologous recombination. In this instance, it is not necessary to employ an expression vector comprising a replication origin suitable for appropriate host cells in order to propagate and express the hsp in the host cells.

A variety of expression vectors may be used including, but not limited to, plasmids, cosmids, phage, phagemids or modified viruses. Typically, such expression vectors comprise a functional origin of replication for propagation of the vector in an appropriate host cell, one or more restriction endonuclease sites for insertion of the hsp gene sequence, and one or more selection markers. The expression vector must be used with a compatible host cell which may be derived from a prokaryotic or an eukaryotic organism including but not limited to bacteria, yeasts, insects, mammals and humans.

For long term, high yield production of properly processed hsp or hsp-peptide complexes, stable expression in mammalian cells is preferred. Cell lines that stably express hsp or hsp-peptide complexes may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while hsp is expressed continuously.

The recombinant cells may be cultured under standard conditions of temperature, incubation time, optical density and media composition. However, conditions for growth of recombinant cells may be different from those for expression of hsps and antigenic proteins. Modified culture conditions and media may also be used to enhance production of the hsp. For example, recombinant cells containing hsps with their cognate promoters may be exposed to heat or other environmental stress, or chemical stress. Any techniques known in the art may be applied to establish the optimal conditions for producing hsp or hsp-peptide complexes.

5.1.8 Peptide Synthesis

An alternative to producing hsp by recombinant techniques is peptide synthesis. For example, an entire hsp, or a peptide corresponding to a portion of an hsp can be synthesized by use of a peptide synthesizer. Conventional peptide synthesis or other synthetic protocols well known in the art may be used.

Peptides having the amino acid sequence of a hsp or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting hsp is accomplished using conventional procedures, such as preparative HPLC using gel permeation, partition and/or ion exchange chromatography. The choice of appropriate matrices and buffers are well known in the art and so are not described in detail herein.

5.2 Method of Determining ATPase Activity

According to the invention, the ATPase activity of a hsp or hsp-peptide complex can be used to correlate and thus determine the biological activity of the hsp or hsp-peptide complexes. The methods of the invention can be used to detect in a sample the presence or absence of hsp or hsp-peptide complex that is biologically active. The method can be quantitative such that when the mass of hsp or hsp-peptide complex in a sample is known, a specific biological activity can be derived. The specific biological activity of a sample of hsp or hsp-peptide complex can be used to estimate the therapeutic or prophylactic amount of a sample. The specific activity can also be used to compare the biological activities of samples containing hsp or hsp-peptide complex. The method can further comprise determining the ATPase activity in the presence of geldanamycin or any other specific inhibitors of nucleotide binding to hsps (e.g., the adenosine analog 5′-N-ethylcarboaxamido-adenosine (NECA)), wherein the ATPase activity that is inhibited by the presence of geldanamycin, or any other specific inhibitors of nucleotide binding, is correlated with the biological activity. This step is particularly useful when other types of ATPase may be present in a sample.

Any method that can measure ATPase activity can be used. Non-limiting examples include enzyme reactions that involve the generation or consumption of ATP, ADP, and/or AMP, such as but not limited to assays based on light-emitting enzymes, and methods that detect the concentrations and/or quantities of ATP, ADP, AMP and/or inorganic phosphate, such as but not limited to ion exchange chromatography.

In one particular embodiment, a bioluminsecence ATP assay can be used. By way of example, and not limitation, the assay may be performed as follows:

Protein samples are concentrated using Millipore BIOMAX spin columns (10 Kda MWCO) to a protein concentration of 500 μg/ml. Gp96 (1 μg) is mixed with a solution of ATP (10-30 fold molar excess) containing either 20% DMSO or 20% DMSO containing 400 μM of the specific inhibitor geldanamycin. The mixture is then incubated at 37° C. for a period of 4 hours.

Subsequently, individual samples are diluted to 100 μL with PBS. The ATP contained in 50 μL of each sample is quantified by comparison to an ATP standard curve using a Molecular Devices Lmax luminometer and ROCHE CLII bioluminescence kit. The differential in ATP concentration between DMSO and DMSO/Geldanamycin measurements represents the amount of ATP hydrolysis specific to gp96. As an alternative, geldanamycin can be replaced with NECA in this assay.

In another embodiment, an ion exchange chromatography method can be used. By way of example, and not limitation, the assay may be performed as follows:

Gp96 samples at 0.5 mg/mL are incubated with a 10× molar ration of ATP at 37° C. in the presence of 5 mM MgCl₂ and 25 mM KCL in PBS. Aliquots are taken at various time points and the remaining ATP is determined using an ion exchange HPLC system. For geldanamycin inhibition experiments, geldanamycin is added to the mixture at a ratio to ATP of 1:1, 2:1, 5:1, and 10:1. Aliquots are taken at various time points for ATP analysis.

ATP hydrolysis by casein kinase II is tested under the following conditions: 0.5 U/mL casein kinase II is mixed with 20 μg/mL ATP, 2 mg/mL casein with or without geldanamycin (geldanamycin to ATP ratios of 1:1, 1:2, 1:5 and 1:10) and incubated at 37° C. in 40 mM HEPES containing 10 mM MgCl₂, 130 mM KCL and 5 mM DTT. Aliquots are taken at various time points and analyzed for ATP concentration.

ATP concentration is measured by ion exchange HPLC using a Partisil SAX column (2.1×250 mm, 10 μm, Alltech) and elution is carried out at 0.25 mL/min using a linear gradient from 0.3-1 M ammonium phosphate in 25 minutes. All chromatography is performed on a Waters Alliance HPLC system with absorbance detection at 215 nm. The ATP is completely separated form ADP and AMP, and quantification is based on peak area using an ATP standard curve. The specific ATPase activity for gp96 is expressed as the amount of ATP hydrolyzed per mg of gp96 protein per hour (nmol/mg/hr). The specific ATPase activity of casein kinase II is expressed as the amount of ATP hydrolyzed per unit of protein per hour (nmol/U/hr). All rate values are corrected for background hydrolyis as determined for samples containing ATP alone without added gp96 or casein kinase II.

ATPase activity can also be measured using the immunoaffinity stripping technique, which uses [α-³²P]-labeled ATP and thin layer chromatography, presented by way of example and not limitation, is as follows. (Li and Srivastava (1993) EMBO J. 12:3143; Wearsch and Nicchitta (1997) J. Biol. Chem. 272:5152):

Typically, 1 μg of purified gp96 or hsp70 is incubated with 20 μM [γ-³²P]ATP in a reaction volume of 20 μl containing 20 mM HEPES, pH 7.2, 20 mM NaCL and 2 mM MgCl₂ at 37° C. for 1 h. 1 μl of the reaction mixture is then spotted onto a polyethyleneimine (PEI) cellulose plate. Thin layer chromatography is performed against 1:1 ratio of 1M LiCl and 1M HCOOH.

The plate is then dried, exposed to film and corresponding radioactive spots are excised and counted. ATPase activity is determined from the amount of [α-³²P]ADP and [α-³²P]AMP generated from [γ-³²P]ATP, i.e. the percentage of ATP hydrolyzed calculated as [ADP+AMP]/[ATP+AMP+ADP]×100%. Background ATP hydrolysis lacking purified gp96 or hsp70 is subtracted.

ATPase activity can also be measured using HPLC. The following is presented by way of example and not limitation:

Gp96 (2 μg; 200 μg/ml in PBS) is added to an equal volume of PBS containing ATP (160 μM), 10 mM MgCl₂ and 20% DMSO. In control experiments, geldanamycin (160 μM) dissolved in DMSO replaces DMSO alone. Samples are mixed and incubated for 4 hours at 37° C. Following this, 80 μL of 100 mM KH₂PO₄ (pH 4.0) is added to quench the reaction and the samples are loaded into an HPLC auto sampler for injection. ADP is separated from ATP using an Aquasil C18 column (250 mm×4.6 mm) equilibrated in 60 mM KH₂PO₄ (pH 6.0) containing 1% Acetonitrile. Flow rate is 0.5 ml/min. The amount of ADP is quantified following automated peak integration and comparison to an ADP standard curve.

Radioisotopic assays known in the art can also be used to determine the ATPase activity of hsp or hsp-peptide-complex.

5.3 Method of Determining Oligomeric Structure by Size

According to the invention, the oligomeric structure of an hsp or hsp-peptide complex can be used to determine the biological activity of the hsp or hsp-peptide complexes. The methods of the invention can be used to detect in a sample the presence or absence of hsp or hsp-peptide complex that is biologically active. The method can be quantitative such that when the mass of hsp or hsp-peptide complex in a sample is known, a specific biological activity can be derived. The specific biological activity of a sample of hsp or hsp-peptide complex can be used to estimate the therapeutic or prophylactic amount of a sample. The specific activity can also be used to compare the biological activities of samples containing hsp or hsp-peptide complex.

Any method known in the art for detecting or measuring the presence of oligomeric structure can be used. Preferably, the method is designed to be used with hsp. Methods that monitor the progress of oligomerization can also be used.

In one particular embodiment, size exclusion chromatography can be used. A procedure, described by Chadli et al., (1999) J. Biol. Chem. 274:4133-4139, presented by way of example and not limitation, is as follows:

Size exclusion chromatography, using a FPLC system (Amersham Pharmacia Biotech), is carried out at 4° C. on superose 6 or Superose 12 10/30 columns equilibrated with 50 mM Tris-HCL, pH 8.65. Elution is performed using the same buffer at a flow rate of 0.2 ml/min. The eluted proteins are detected by the UV absorbance at 280 nm. The colums are calibrated with high and low molecular mass calibration kit from Amersham Pharmacia Biotech. The standards used are thyroglobulin (669 kDa), ferritin (440 kDa), catalase (232 kDa), aldolase (158 kDa), bovine serum albumin (67 kDa) and chymotrypsinogen (25 kDa). Blue dextran and potassium bichromate are used to determine the void volume and the total volume, respectively.

In another embodiment, SDS and Native PAGE, can be used. A procedure described by Chadli, et al. (1999) J. Biol. Chem. 274:4133-4139, presented by way of example and not limitation, is as follows:

SDS and Native PAGE and silver staining are performed on a Phast system (Amersham Pharmacia Biotech). The molecular mass calibration kit (Amersham Pharmacia Biotech) includes phosphorylase b (94 kDa), bovine serum albumin (67 kDa), ovalubumin (43 kDa), carbonic anhydrase (30 kDa), soybean trypsin inhibitor (20 kDa) and α-lactalbumin (14.4 kDa).

Native PAGE is performed at 4° C. on Multipore II (Amersham Pharmacia Biotech) using 10% polyacrylamide Clean gel, 375 mM Tris buffer, pH 8.9. A calibration kit (Amersham Pharmacia Biotech) containing thyrglobulin (669 kDa), ferritin (440 kDa), catalase (232 kDa), lactate dehygrogenase (140 kDa) and bovine serum albumin (67 kDa) is used to estimate the apparent molecular mass of proteins accroding to the manufacturer's recommendations. Scanning of silver stained bands is performed on an Omni media scanner apparatus using Bio-image software (Millipore).

Hsp samples resolved on native PAGE are electrotransferred to nitrocellulose. Air-dried filters are incubated 2 h in 10% nonfat dry milk in PBS, 0.05% Tween 20 (PBST), washed with PBST, incubated for 2 h with anti-hsp90 antibodies Ab119 (37) (1:4000) or d7a (28) (1:4000), washed with PBST, incubated for 1 h with anti-rabbit or anti-mouse antibodies, washed with PBST, and revealed utilizing ECL (Amersham Pharmacia Biotech).

In another embodiment, gel filtration chromatography, can be used. A procedure described by Wearsch and Nicchitta (1996) Biochem. 35:16760-9, presented by way of example and not limitation, is as follows:

A TSK-GEL g3000 SW analytical gel filtration column (Tosohaus, Montgomeryville, Pa.) is equilibrated in either PBS (phosphate buffered saline) or buffer A (110 mM KOAc, 25 nM K-HEPES, pH 7.2, 20 mM NaCl, 1 mM Mg(OAc)₂, 0.1 mM CaCl₂). Samples in a volume of 125 μL are injected onto the colums at a lfow rate of 0.6 mL/min. Fractions of 0.5 mL are collected and analyzed by SDS-PAGE. The Stokes radius (Rₛ) for each sample is determined by comparison with standards (thryroglobulin, Rₛ=8.5 nm, 660 kDa; apoferritin, Rₛ=3.5 nm, 66 kDa; ovalbumin, Rₛ=3.1 nm, 43 kDa). Where indicated, grp94 is incubated with 4 mM Zwittergent 3-12 for 45 min at RT in 125 μL of buffer A or 1×PBS prior to analysis.

In yet another embodiment, two-dimensional electrophoresis, can be used. A procedure described by Wearsch and Nicchitta (1996) Prot. Exp. Pur. 7:114-121, presented by way of example and not limitation, is as follows:

For the first (non-reducing) dimension, samples are placed in 0.5 M Tris, 5% SDS, 0.01% bromophenol blue and heated for 10 min at 55° C. Two 1-μg samples of purified grp94 are prepared, one which inludes 50 mM DTT in the sample buffer. Five micrograms of high-molecular weight standards and IgG are included as controls. Samples are loaded on 7.5% gels cast in capillary tubes and run at 200 V in the Mini-Protean II 2-D Cell (Bio-Rad). The tube gels are extracted, incubated in 0.5 M Tris, 5% SDS, 0.1 M DTT for 15 min at 55° C., overlaid on 7.5% preparative slab gels, and run in the second dimension. Proteins are visualized by Coomasie blue staining.

Velocity sedimentation can also be used to determine the multimeric structure of an hsp or hsp-peptide complex. The following assay, as performed by Wearsch and Nicchitta (1996) Biochem. 35:16760-9, is presented by way of example, and not limitation:

For the determination of sedimentation coefficients (s), samples are analyzed on sucrose gradients in parallel with known standards. Native grp94, elastase-digested grp94, and in vitro synthesized grp94-AC (see Construct Preparation) are analyzed on gradients of 15-30% sucrose supplemented with buffer A. In vitro synthesized grp94-Cexp translation products and grp94-MBP fusion proteins are analyzed on 10-25% sucrose gradients supplemented with PBS. Gradients of 11.5 mL are prepared and harvested with an Auto Densi-Flow IIc (Buchler Instruments, Lenexa, Kans.). Samples in a volume of 200 μL are loaded onto gradients and centrifuged in the Beckman SW41 rotor at 40,000 rpm for 20 h at RT. Catalase (11.3 S), yeast alcohol dehydrogenase (7.4 S), BSA (4.3 S), ovalbumin (3.5 S), and chymotrypsinogen A (2.6 S) are employed as standards. Fractions of 0.5 mL are collected from gradients and analyzed by SDS-PAGE (grp94 samples) or by the absorbance at 280 nm (protein standards).

Mass spectroscopy can also be used by anyone in the art to determine the oligomeric structure of gp96 and/or gp 96 complex.

In another embodiment, given by way of example and not limitation, a filter can be used to determine the presence and/or quantity of gp96 dimer and/or gp96 complex dimer. In a preferred embodiment, such a filter is a molecular weight cutoff filter, which allows molecules of a certain size to pass while larger molecules are trapped.

Another embodiment of the invention uses a light scattering assay to determine the presence or concentration of the oligomeric form of the gp96 and/or gp96 complex.

In yet another embodiment, analytical ultracentrifugation can be used to determine the presence and/or quantity of gp96 dimer and/or gp96 complex dimer. This technique is well known to the skilled artisan.

In another embodiment, gradient centrifugation can be used to determine the presence and/or quantity of gp96 dimer and/or gp96 complex dimer. This technique is well known to the skilled artisan.

5.4 Methods of Determining Biological Activity

According to the invention, the oligomeric structure or the ATPase activity of an hsp and/or hsp-peptide complex can be used to as a proxy to detect or measure the biological activity of the hsp or hsp-peptide complexes. Biological activity of an hsp and/or hsp-peptide complex that is of particular interest includes, but is not limited to immunological activity, e.g., presentation of peptide antigen to antigen presenting cells (antigen re-presentation) and T-cell activation; induction of production of biological response modifiers, such as but not limited to cytokines, e.g., human macrophage chemo-attractant protein-1 (MCP-1), and nitric oxide (NO); binding of receptors such as CD91 (alpha-2-macroglobulin receptor, α2MR) and/or CD36; binding and release of an antigenic molecule; and the ability to cause the regression of tumors in animals, prolongation of survival of tumor-bearing animals and elimination of infection. The biological activities may occur or be observed in vivo and/or in vitro.

The ability of hsps to bind peptides is apparent, but the mechanism for this peptide loading is not well understood. Gp96 is a protein found in the endoplasmic reticulum and binds a multitude of peptides in vitro and in vivo with no apparent amino acid specificity. Indeed, in order for gp96 to play its role in immune response, peptide must first be bound to the hsp (Sastry and Linderoth (1999) J. Biol. Chem. 274:12023-12035).

Sastry and Linderoth (supra) have described an assay to detect the binding of a peptide to gp96. Briefly, peptide-pyrene is incubated with a molar excess of gp96 for 10 min at room temperature in low salt buffer (20 mM HEPES, pH 7.9, 20 mM NaCl, 2 mM MgCl₂). The mixture is dialyzed using a 70,000 molecular weight cutoff membrane for 2 h to remove any unbound peptide-pyr. The flouresence of the retentate is monitored by excitation at 340 nm. At this wavelength, the protein chromophores (Trp and Tyr) do not absorb UV light, and only the pyrene is excited.

Re-presentation of antigenic peptides by hsps is another biological activity of significance. The inventors have demonstrated that loss of dimeric structure of gp96 correlates with a loss of its antigen re-presentation activity. Immune response mediated by RAW264.7 APCs and CTLs specific to the peptide of the gp96 complex was measured by IFN-γ levels secreted by the CTLs. IFN-γ is a cytokine that is known in the art to be an indicator of general immune status; it also plays a major role in tumor recognition and rejection. Dimeric gp96 was able to stimulate secretion of IFN-γ in a dose-dependent manner, while aggregated gp96 did not stimulate secretion of IFN-γ. A re-presentation assay is described in section 8, by way of example and not limitation.

In response to being presented with hsp or hsp-peptide complexes, antigen presenting cells secrete MCP-1 and produce NO. MCP-1 plays an important role in the inflammatory response of blood monocytes and tissue macrophages. NO has also been shown to be a significant modulator of immune response (Wei et al., (1995) Nature 375:408-411).

The CD91/α2M receptor is a cell surface receptor for heat shock proteins. The CD91/α2M receptor plays a role in endocytosis of a diversity of ligands. In addition to α2M, other ligands of α2MR include lipoprotein-peptide complexes, lactoferrin, tissue-type plasminogen activator (tPA), urokinase-type plasminogen activator (uPA), and exotoxins. Thus, the α2M receptor plays roles in a variety of cellular processes, including endocytosis, antigen presentation, cholesterol regulation, ApoE-containing lipoprotein clearance, and chylomicron remnant removal. Thus, the methods of the invention that may allow the detection or measurement of the ability of hsp or hsp-peptide complex to bind or block binding of other molecules to CD91/α2M receptor.

CD36 has also been shown to act as a receptor for heat shock proteins. CD36 is a member of the class B scavenger receptor family and is primarily expressed in capillary endothelial cells, mammary secretory epithelial cells, differentiated adipose cells, B cells, macrophages, and several types of tumor cells. CD36 is believed to play a role in platelet adhesion and aggregation, phagocytosis of apoptotic cells, and in the metabolism of long-chain fatty acids. In particular, the heat shock protein gp96 has been shown to bind to CD36 and stimulate the production of nitric oxide and chemokine in these cells. (Panjwani et. al., 2000, Cell Stress & Chaperones 5 (4):373-397; U.S. Provisional Application No. 60/238,865, filed on Oct. 6, 2000). Thus, the methods of the invention may allow the detection or measurement of the ability of hsp or hsp-peptide complex to bind or block binding of other molecules to CD36.

5.5 Uses in Quality Control and Standardization

According to the invention, the oligomeric structure or the ATPase activity of an hsp or hsp-peptide complex can be used to determine the biological activity of the hsp or hsp-peptide complexes. The methods of the invention can be used to detect in a sample the presence or absence of hsp or hsp-peptide complex that is biological active. The method can be quantitative such that when the mass of hsp or hsp-peptide complex in a sample is known, a specific biological activity can be derived. The specific biological activity of a sample of hsp or hsp-peptide complex can be used to estimate the therapeutic or prophylactic amount of a sample. The specific activity can also be used to compare the biological activities of samples containing hsp or hsp-peptide complex.

The methods of the invention can be used in the quality control of a preparation of hsp-peptide complexes during their commercial production and during storage. The quality aspects of a preparation of hsp-peptide complexes encompass the potency of the preparation which can be related to its specific activity; the stability of the preparation which relates to storage requirements, storage history and its impact on potency. The methods of the invention can also be used to determine or predict the number of doses that can be derived from a given preparation of hsp or hsp-peptide complexes, especially in the context of patient-specific preparations of hsp-peptide complexes.

Hsps and complexes thereof have many commercial uses. The use of hsp-peptide complexes for the treatment and prevention of cancer and infectious diseases has been described in U.S. Pat. Nos. 6,030,618; 5,935,576; 5,750,119; 5,961,979; 6,048,530; 5,837,251; and 6,017,540. The use of stress protein-antigen complexes for sensitizing antigen presenting cells in vitro for use in adoptive immunotherapy is described in PCT publication WO 97/10002, dated Mar. 20, 1997 (see also U.S. Pat. No. 5,985,270 issued Nov. 16, 1999). The methods of the invention are also useful in methods for improving formulations and efficacies of treatment comprising hsp or hsp-peptide complexes, such as that described in U.S. Provisional Application No. 60/232,779; and U.S. patent application Ser. No. 09/693,643, which are incorporated herein by reference in their entireties.

The methods of the invention can be used to replace the relatively expensive and cumbersome biological assays that are described in section 5.5. The methods of the invention can be used in monitoring the compliance with regulatory requirements in a manufacturing process.

5.6 Uses in Diagnosis and Prognosis

In one embodiment, the invention provides a method for diagnosing a condition in a subject that is due in part to the functions of the subject's immune system, said method comprising correlating the ATPase activity of heat shock protein or heat shock protein-peptide complex or the presence of the dimeric form of heat shock protein or heat shock protein-peptide complex obtained from the subject with a biological activity of the heat shock protein or heat shock protein-peptide complex. Since the biological activity is associated with one or more immune functions in the subject, a change in ATPase activity or the amounts of dimeric form indicates a change in the condition. The methods of the invention provide a convenient way to monitor the general state of health of the immune system, the non-antigenic specific aspects of the immune system, and/or the immune responsiveness of a subject.

In another embodiment, the invention provides a method for determining the prognosis of a cancer or an infectious disease in a subject comprising correlating the ATPase activity of heat shock protein or heat shock protein-peptide complex or the presence of the dimeric form of heat shock protein or heat shock protein-peptide complex obtained from the subject with a biological activity of the heat shock protein or heat shock protein-peptide complex, wherein the biological activity is associated with one or more immune functions that is responsive to or target the cells of the cancer or the agents that cause the infectious disease. Such immune functions relate to antigen presentation, amplification of the response to an antigen, as well as effector cell functions in both specific and non-specific aspects. Thus, a change in ATPase activity or the amounts of dimeric form indicates a change in the prognosis.

In yet another embodiment of the invention, the prognosis of cancer or an infectious disease can be established by measuring the ATPase activity and/or the multimeric structure of hsp and/or hsp-peptide complex isolated from the cancer cells or infected cells from a subject. The biological activity of hsp or hsp-peptide complex produced by such cells reflects the in vivo immunogenicity of the cancer cells or infected cells, and can be used to provide the prognosis of cancer or an infectious disease.

In various embodiments, the methods of the invention can also be used to monitor a subject's immune status after receiving treatment such as a vaccine, chemotherapy, radiation, or immunotherapy.

5.7 Modulating Bioactivity of HSPs and Hsp-Peptide Complexes

In yet another embodiment, the invention provides a method for modulating the biological activity of heat shock protein and complexes thereof comprising contacting the heat shock protein and complexes with compounds that modulate the ATPase activity or the oligomerization of the heat shock protein and complexes. Such compounds can be used to modulate the immune functions of a subject, and can be identified by the methods of the invention as described below.

The modulation of the bioactivity of hsp or hsp-peptide complex can be used to treat either immunodeficient conditions (such as but not limited to use of immunosuppressive drugs, chemotherapy and radiation exposure), or conditions associated with an overactive immune system (such as but not limited to allergy, transplantation rejection, and autoimmune diseases).

5.8 Drug Screening

The invention can also be used to screen for therapeutic agents that modulate the biological activities of hsp or hsp-peptide complexes. In one embodiment, an agent can be screened by contacting it with a composition comprising an hsp or hsp-peptide complex, and then determining the ATPase activity or the dimerization of the hsp or hsp-peptide complex in the composition.

An increase in ATPase activity or dimers relative to the uncontacted composition would identify the test compound as a therapeutic agent that increases biological activities.

A decrease in ATPase or dimers, compared to the uncontacted composition would identify the test compound as a therapeutic agent that decreases biological activities.

Examples of therapeutic agents include but are not limited to peptides, small molecules, peptide mimetics, antibodies, lipids, carbohydrates, antibiotics, including geldanamycin analogs (e.g., 17-ally-amino-geldanamycin); and nucleotide analogs (see Rosser et al., J. Bio. Chem. 2000, 275:22789).

Therapeutic agents can also be tested to see if they increase or decrease the resistance of the hsp to agents or environmental factors which increase or decrease the bioactivity of the hsp. This embodiment involves first, measuring the bioactivity of an hsp in a composition by ATPase activity or detection of dimers, second, contacting the agent to be screened with the composition comprising the hsp and then measuring biological activities again, and finally contacting the composition with an agent or changing an environmental factor, for example pH or temperature, which is known in the art to inhibit or promote biological activities, and then measuring biological activities for a third time. In this way it can be determined if the screened agent modulates the biological activities of an hsp by increasing or decreasing the effect of a known inhibitor or promoter of hsp biological activities.

The methods of the invention can also be used to screen for ways to modulate hsp biological activity. Compounds can be added and removed from compositions containing an hsp-peptide complex, with the bioactivity of the hsp-peptide complex measured both before and after the addition or the compound. Compounds which can be added or subtracted from the composition include, but are not limited to ions, acids, bases, salts, metals, gases, liquids, solids, enzymes, proteins, lipids, carbohydrates, peptides and nucleotides. Compounds can also be tested to determine if they increase or decrease the resistance of the hsp-peptide complex to agents or environmental factors which are known in the art to increase or decrease the bioactivity of the hsp-peptide complex.

5.9 Kits for Determining Biological Activity

The invention also provides kits for carrying out the methods and/or therapeutic regimens of the invention. One embodiment, given by way of example and not limitation, would be a kit comprising a container of an hsp or hsp-peptide complex of known specific activity as a positive control, a container of ATP and a container of geldanamycin. Another embodiment, given by way of example and not limitation, would be a kit comprising a container of an hsp or hsp-peptide complex of known specific activity as a positive control and conformation-specific antibodies to gp96. In another embodiment, given by way of example and not limitations, is a kit comprising a container of an hsp or hsp-peptide complex of known specific activity as a positive control and a molecular weight cutoff filter. Also included in the kits of the invention is instruction for determining the ATPase activity or the amount of the dimeric form of the heat shock protein or heat shock protein-peptide complex.

5.10 Rational Design of Recombinant HSPs

The methods of the invention can also be used to screen variants hsps for increased or decreased bioactivity. Variants can be created by one skilled in the art using recombinant DNA techniques including mutagenesis, error prone PCR, gene shuffling. Once the recombinant hsp is obtained, the recombinant hsp and complexes can be measured for bioactivity using the methods of the invention.

5.11 Conformation-Specific Antibodies Against HSP

In yet another embodiment, the invention provides methods for detection of the oligomeric form of an hsp or a specific conformation of an hsp or hsp-peptide complex by the use of conformation-specific polyclonal or monoclonal antibodies, including but not limited to recombinant antibodies, fragments and derivatives thereof. In one embodiment, such antibodies only bind hsp and/or hsp-peptide complex of a particular conformation, e.g., dimeric form, and do not bind other forms of hsp and/or hsp-peptide complex. The bound antibodies can be quantified by techniques known in the art, e.g., ELISA. In specific embodiments, the antibodies bind monomeric and oligomeric (non-dimer) hsp and/or hsp-peptide complex, but do not bind the dimeric form. In yet another embodiment, the antibodies bind an epitope which is present on the monomeric form of hsp or hsp-peptide complex, wherein said epitope is masked when the hsp or hsp-peptide complex oligomerizes or dimerizes.

Polyclonal and/or monoclonal antibodies that are conformation specific can be created by techniques well known in the art. In this example, the antibodies are specific to the dimeric form of the hsp or hsp-peptide complex to be measured.

The conformation-specific antibodies can also be used to isolate or concentrate hsp or hsp-peptide complex that display biological activity. Any method known in the art, such as affinity purification, can be used. This allows the preparation of compositions comprising highly potent hsp or hsp-peptide complex.

Any method known in the art for detection conformational changes in hsps or hsp-peptide complexes can be used, including by way of example and not limitation, intrinsic fluorescence, circular dichroism, calorimetry or assays that determine ligand binding that differs with protein conformation.

5.12 Antigenic Molecules

The following subsections provide an overview of peptides that are useful as antigenic/immunogenic components of the hsp-peptide complexes of the invention, and how such peptides can be identified, e.g., for use in recombinant expression of the peptides for in vitro complexing of hsps and antigenic molecules. However, in the practice of the present invention, the identity of the antigenic molecule(s) of the hsp/peptide-complex need not be known, for example when the hsp/peptide complexes can be purified as a population of complexes directly from a cancerous cell or from a tissue infected with a pathogen.

In one embodiment, the antigenic peptides can be derived from cancer cells or cells infected by a pathogen or infectious agent that causes the infectious disease. Examples of pathogens or infectious agents, include, but are not limited to, a virus, bacterium, fungus, protozoan, parasite, etc. Preferably, the pathogen is one that infects humans. The antigenic peptides can be generated by proteolytic digestion of proteins (e.g., cytosolic and/or membrane-derived proteins) obtained from cancer cells, infected cells or antigenic cells that share antigenic determinants with or display similar antigenicity as the cancer cells, infected cells, or the pathogens including viral particles. The antigenic peptides can also be generated by exposing the proteins to ATP, guanidium hydrochloride, and/or acid. The antigenic peptides can also be generated from antigenic cells that display the antigenicity of an agent (pathogen) that causes the infectious disease, or a variant of such agent.

Since whole cancer cells, infected cells or other antigenic cells are used in the present methods, it is not necessary to isolate or characterize or even know the identities of these antigenic peptides in advance of using the present methods. The source of the antigenic cells may be selected, depending on the nature of the disease with which the antigens are associated. In one embodiment of the invention, any tissues, or cells isolated from a cancer, including cancer that has metastasized to multiple sites, can be used as an antigenic cell in the present method. For example, leukemic cells circulating in blood, lymph or other body fluids can also be used, solid tumor tissue (e.g., primary tissue from a biopsy) can be used. A non-limiting list of cancers, the cells of which can be used herein is provided in Section 5.18 below.

In another embodiment of the invention, any cell that is infected with a pathogen or infectious agent, i.e., an infected cell, can be used as an antigenic cell for the preparation of antigenic peptides. In particular, cells infected by an intracellular pathogen, such as a virus, bacterium, fungus, parasite, or protozoan, is preferred. An exemplary list of infectious agents that can infect cells which can be used herein is provided in Section 5.18.

In yet another embodiment, any pathogen or infectious agent that can cause an infectious disease can be used as antigenic cell for the preparation of antigenic peptides. Variants of a pathogen or infectious agent, such as but limited to replication-defective variants, non-pathogenic or attenuated variants, non-infectious variants, can also be used as an antigenic cell for this purpose. For example, many viruses, bacteria, fungi, parasites and protozoans that can be cultured in vitro or isolated from infected materials can serve as a source of antigenic cells. Methods known in the art for propagating such pathogens including viral particles can be used. An exemplary list of pathogens or infectious agents that can be used as antigenic cells is provided in Section 5.18.

Cell lines derived from cancer tissues, cancer cells, or infected cells can also be used as antigenic cells. Cancer or infected tissues, cells, or cell lines of human origin are preferred. Cancer cells, infected cells, or antigenic cells can be identified and isolated by any method known in the art. For example, cancer cells or infected cells can be identified by morphology, enzyme assays, proliferation assays, or the presence of pathogens or cancer-causing viruses. If the characteristics of the antigens of interest are known, antigenic cells can also be identified or isolated by any biochemical or immunological methods known in the art. For example, cancer cells or infected cells can be isolated by surgery, endoscopy, other biopsy techniques, isolation from body fluids (such as blood), affinity chromatography, and fluorescence activated cell sorting (e.g., with fluorescently tagged antibody against an antigen express by the cells). Antigenic cells that display similar antigenicity have one or more antigenic determinants in common against which an immune response in a subject is desired (e.g., for therapeutic or prophylactic purposes).

Preferably, when used to treat or prevent cancer, known tumor-specific (i.e., expressed in tumor cells) or tumor associated antigens (i.e., relatively overexpressed in tumor cells) or fragments or derivatives thereof are used. For example, such tumor specific or tumor-associated antigens include but are not limited to KS 1/4 pan-carcinoma antigen (Perez and Walker, 1990, J. Immunol. 142:3662-3667; Bumal, 1988, Hybridoma 7 (4):407-415); ovarian carcinoma antigen (CA125) (Yu, et al., 1991, Cancer Res. 51 (2):468-475); prostatic acid phosphate (Tailer, et al., 1990, Nucl. Acids Res. 18 (16):4928); prostate specific antigen (Henttu and Vihko, 1989, Biochem. Biophys. Res. Comm. 160 (2):903-910; Israeli, et al., 1993, Cancer Res. 53:227-230); melanoma-associated antigen p97 (Estin, et al., 1989, J. Natl. Cancer Inst. 81 (6):445-446); melanoma antigen gp75 (Vijayasardahl, et al., 1990, J. Exp. Med. 171 (4):1375-1380); high molecular weight melanoma antigen (Natali, et al., 1987, Cancer 59:55-63) and prostate specific membrane antigen. Other exogenous antigens that may be complexed to hsps include portions or proteins that are mutated at a high frequency in cancer cells, such as oncogenes (e.g., ras, in particular mutants of ras with activating mutations, which only occur in four amino acid residues (12, 13, 59 or 61) (Gedde-Dahl et al., 1994, Eur. J. Immunol. 24 (2):410-414)) and tumor suppressor genes (e.g., p53, for which a variety of mutant or polymorphic p53 peptide antigens capable of stimulating a cytotoxic T-cell response have been identified (Gnjatic et al., 1995, Eur. J. Immunol. 25 (6):1638-1642).

Preferably, when used to treat or prevent viral diseases, suitable proteins and peptides comprising epitopes of known viruses can be expressed in the appropriate cells. For example, such antigenic epitopes from viruses include, but not limited to, hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, smallpox virus, rubella virus, polio virus, human immunodeficiency virus type I (HIV-I), and human immunodeficiency virus type II (HIV-II).

Preferably, when used to treat or prevent bacterial infections, suitable proteins and peptides comprising epitopes of known bacteria can be expressed in the appropriate cells. For example, such bacterial epitopes may be derived from various bacteria including, but not limited to, Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease, and Leptospira), anaerobic bacteria (e.g., Actinomyces and Clostridium species including C. tetani, C. botulinum, C. perfringens), Gram positive and negative coccal bacteria, Streptococcus species, Pneumococcus species, Staphylococcus species (e.g., S. aureus and S. pneumonia), Neisseria species (e.g., N. meningitidis).

Preferably, when used to treat or prevent fungal infections, suitable proteins and peptides comprising epitopes of known fungi can be expressed in the appropriate cells. For example, such antigenic epitopes may be derived from various fingi including, Aspergillus (e.g., Aspergillus fumigatus), Cryptococcus (e.g., Cryptococcus neoformans), Sporotrix, Coccidioides, Paracoccidioides, Histoplasma, Blastomyces, Candida (e.g., Candida albicans), Rhizopus, Rhizomucor, Absidia, and Basidiobolus species.

Preferably, when used to treat or prevent parasitic infections, suitable proteins and peptides comprising epitopes of known protozoa, nematodes, or helminths can be expressed in the appropriate cells. For example, such antigenic epitopes may be derived from various protozoa including, but not limited to, Entoamoeba, Plasmodium, Leishmania, Eimeria, Cryptosporidium, Giardiasis, Toxoplasma, and Trypanosoma species.

5.12.1 Preparation of Antigenic Proteins

In one embodiment of the invention, a protein preparation is provided which is derived from a cancer cell, infected cell, or pathogen. For example, for the treatment of cancer, the protein preparations are prepared, postoperatively, from tumor cells obtained from a cancer patient. In another embodiment of the present invention, one or more antigenic proteins of interest are synthesized in cell lines modified by the introduction of recombinant expression systems that encode such antigens, and such cells are used to prepare the proteins. The proteins can be obtained from one or more cellular fraction(s), for example, the cytosol of the antigenic cells, or they can be extracted or solubilized from the membranes or cell walls of the antigenic cells. Any technique known in the art for cell lysis, fractionation of cellular contents, and protein enrichment or isolation can be used. See, for example, Current Protocols in Immunology, vol. 2, chapter 8, Coligan et al. (ed.), John Wiley & Sons, Inc.; Pathogenic and Clinical Microbiology: A Laboratory Manual by Rowland et al., Little Brown & Co., June 1994; which are incorporated herein by reference in their entireties. Depending on the techniques used to fractionate the cellular contents, a cellular fraction comprises at least 20, 50, 100, 500, 1,000, 5,000, 10,000, or 20,000 different proteins.

As used herein, the term “protein preparation” refers to a mixture of proteins obtained from antigenic cells, a cellular fraction of antigenic cells, or virus particles. The proteins can be obtained from a cellular fraction, such as the cytosol. The proteins can also be non-cytosolic proteins (e.g., those from cell walls, cell membranes or organelles), or both. Cellular fractions can include, but are not limited to, cytosolic fractions, membrane fractions, and organelle fractions, such as nuclear, mitochondrial, lysosomal, and endoplasmic reticulum-derived fractions. The protein preparations can be obtained from non-recombinant or recombinant cells.

In a specific embodiment, the protein preparations have not been subjected to any method of preparation that selectively removes or retains one or more particular protein(s) from the other proteins in the antigenic cells.

In a specific embodiment, the protein preparation is the total cell lysate which is not fractionated and/or purified, and may contain other non-proteinaceous materials of the cells.

In another specific embodiment, the protein preparation is the total protein in a cellular fraction, which has not been subjected to further fractionation or purification, and may contain other non-proteinaceous materials of the cells.

In yet another embodiment, the protein preparation is the total protein in a preparation of viral particles.

In specific embodiments, the protein preparation comprises total cellular protein, total cytosolic proteins, or total membrane-bound proteins of antigenic cell(s).

In various embodiments, the protein preparation comprises at least 20, 50, 100, 500, 1,000, 5,000, 10,000, or 20,000 different proteins. A plurality of different antigenic proteins are present in a protein preparation of antigenic cells. Moreover, the proteins in the protein preparation may be subjected to a step of protease digestion prior to in vitro complexing to hsps. Alternatively, the proteins in the protein preparation are not subjected to a step of protease digestion prior to in vitro complexing to hsps.

To make a protein preparation of antigenic cells or virus particles, the lysing of antigenic cells or disruption of cell walls, cell membranes, or viral particle structure can be performed using standard protocols known in the art. In various embodiments, the antigenic cells can be lysed, for example, by mechanical shearing, sonication, freezing and thawing, adjusting the osmolarity of the medium surrounding the cells, or a combination of techniques. In other embodiments, the antigenic cells can be lysed by chemicals, such as detergents.

Once the cells are lysed, cellular debris, materials that are non-proteinaceous or materials that do not contain cytosolic, and/or membrane-derived proteins (including proteins in the membranes of organelles) can be removed. Removal of these components can be accomplished by techniques such as low speed centrifugation or filtration. After removing cellular debris and intact cells, a high speed centrifugation step can be used to separate the cytosolic proteins which are in the supernatant, and the membrane-derived proteins which are collected in the pellet. Standard procedures commonly known in the art allows the further isolation of the membrane-derived proteins from the pellet. Standard techniques commonly known in the art can be used to extract viral proteins from viral particles. These separation methods act on the basis of the general and overall size, density, and/or charge of the molecules that are present in the antigenic cell, in the cytosol or in the membranes. These separation methods do not or are not designed to selectively remove or retain any one or more particular protein(s) from other proteins.

In various embodiments, the proteins from the antigenic cells can be optionally separated by their general biochemical and/or biophysical properties, such as size, density, charge, cellular location or combinations thereof. Many techniques known in the art can be used to perform the separation.

An exemplary, but not limiting, method that may be used to make a protein preparation comprising cytosolic proteins is as follows:

Cells, which may be tumor cells derived from a biopsy of the patient or tumor cells cultivated in vitro, or cell infected with a pathogenic agent, are suspended in 3 volumes of 1× Lysis buffer comprising 30 mM sodium bicarbonate pH 7.5, 1 mM PMSF, incubated on ice for 20 minutes and then the hypotonically-swollen cells are homogenized in a dounce homogenizer until >95% cells are lysed. As an alternative to shearing, cells can be sonicated, on ice, until >99% cells are lysed as determined by microscopic examination. When sonication is used, cells are suspended in a buffer such as phosphate buffered saline (PBS) which may comprises 1 mM PMSF, before sonication.

The lysate is centrifuged at 1,000×g for 10 minutes to remove intact cells, nuclei and other cellular debris. The resulting supernatant is recentrifuged at about 100,000×g for about one hour, and the supernatant recovered. The 100,000×g supernatant may be dialyzed for 36 hours at 4° C. (three times, 100 times volumes each time) against PBS or other suitable buffer, to provide the soluble cytosolic proteins of the present invention. If necessary, insoluble material in the preparation may be removed by filtration or low-speed centrifugation.

An exemplary, but not limiting, method that may be used to make a protein preparation comprising membrane-derived proteins is as follows:

Cells, which may be tumor cells derived from a biopsy of the patient or tumor cells cultivated in vitro, or cells infected with a pathogenic agent, are suspended in 3 volumes of 1× Lysis buffer comprising 30 mM sodium bicarbonate pH 7.5, 1 mM PMSF, incubated on ice for 20 minutes and then the hypotonically-swollen cells are homogenized in a dounce homogenizer until >95% cells are lysed. As an alternative to shearing, cells can be sonicated, on ice, until >99% cells are lysed as determined by microscopic examination. When sonication is used, cells are suspended in a buffer such as phosphate buffered saline (PBS) which may comprises 1 mM PMSF, before sonication.

The lysate is then centrifuged at 100,000×g for 10 minutes to collect the cell membranes. Membrane-derived proteins can be dislodged from the lipid bilayer and isolated from the 100,000 g pellet (where the membrane-derived proteins are located) by resuspending the pellet in 5 volumes of PBS containing 1% sodium deoxycholate (without Ca²⁺ and Mg²⁺) and incubated on ice for 1 h. The resulting suspension is centrifuged for 30 min at 20,000 g and the resulting supernatant harvested and dialyzed against several changes of PBS (without Ca²⁺ and Mg²⁺) to remove the detergent. The resulting dialysate is centrifuged for 90 min at 100,000 g and the supernatant purified further. Then calcium and magnesium are both added to the supernatant to give final concentrations of 2 mM. If necessary, insoluble material in the preparation may be removed by filtration or low-speed centrifugation.

In a specific embodiment, the population of cytosolic and/or membrane-derived proteins obtained from antigenic cells can be complexed to hsp directly without protease treatment or any further extraction or selection processes. Alternatively, the proteins can be subjected to protease treatment prior to complexing.

5.12.2 Preparation of Antigenic Peptides

According to the invention, the cytosolic and membrane-derived proteins obtained from antigenic cells can be optionally digested to generate antigenic peptides. In one embodiment, either the cytosolic or the membrane-derived proteins are used in the digestion. In another embodiment, the cytosolic and membrane-derived proteins are combined in the digestion reaction to generate antigenic peptides. In preferred embodiments, the protein preparations that are used in the protease digestion have not been subjected to any method(s) of preparation that selectively remove or retain one or more particular protein(s) from the other proteins in the antigenic cells, or the cytosol or membranes of the antigenic cells.

Various proteases or proteolytic enzymes can be used in the invention to produce from a protein preparation of antigenic cells a population of peptides which comprises antigenic peptides. The enzymatic digestions can be performed either individually or in suitable combinations with any of the proteolytic enzymes that are well known in the art including, but not limited to, trypsin, Staphylococcal peptidase I (also known as protease V8), chymotrypsin, pepsin, cathepsin G, thermolysin, elastase, and papain. Trypsin is a highly specific serine proteinase that cleaves on the carboxyl-terminal side of lysines and arginines. Due to the limited number of cleavage sites, it is expected to leave many MHC-binding epitopes intact. Staphylococcal peptidase I, a serine proteinase, has specificity for cleavage after glutamic and aspartic acid residues. A digestion can be carried out with a single protease or a mixture of proteases. The proteases or proteolytic enzymes used are incubated under conditions suitable for the particular enzyme. Preferably, the enzyme is purified. Non-enzymatic methods, such as cyanogen bromide cleavage, can also be used for generating peptides. The protein preparation to be digested can be aliquoted into a plurality of reactions each using a different enzyme, and the resulting peptides may optionally be pooled together for use. It may not be necessary to completely digest the proteins in the enzymatic reactions. These reactions results in the generation of a diverse and different set of peptides for each protein that is present in the protein preparation. The production of different peptide sets allows for a greater probability of generating antigenic peptides that are capable of inducing an immune response to the antigens in the protein preparation when they are complexed to hsps. In a preferred embodiment, the protein preparation to be digested is aliquoted into two separate reactions and two different proteolytic enzymes are used to produce two different sets of peptides of the proteins present in the protein preparation. Depending on the proteins, enzymes and reaction conditions, undigested proteins may remain in the reactions. In a preferred embodiment, trypsin and Staphylococcal peptidase I are used separately to digest the protein preparation.

In another preferred embodiment, the enzymatic digestion is terminated before the peptides are complexed to hsps. In one embodiment of the invention, inhibitors can be used for terminating an enzymatic digestion. Enzymatic inhibitors that can be used in the invention include, but are not limited to, PMSF, bestatin, amastatin, leupeptin, and cystatin, depending on which enzymes are used in the protein digestion. Inhibitors for most proteases are well known in the art. Alternatively, another method of terminating an enzymatic digestion is by physical removal of the enzyme from the reaction. This can be done by attaching the enzyme of choice to a solid phase, such as a resin or a material that can easily be removed from the reaction by well known methods such as centrifugation or filtration. The protein preparation is allowed to contact or flow across the solid phase for a period of time. Such immobilized enzymes can be purchased commercially or can be produced by procedures for immobilizing enzymes that are well known in the art.

At the end of the digestion reaction, the peptides can optionally be separated from low molecular weight materials, such as dipeptides, or single amino acid residues, in the preparation. Optionally, the peptides can be separated by their general biochemical and/or biophysical properties, such as size, charge, or combinations thereof. Any techniques known in the art can be used to perform the separation.

In another embodiment of the invention, peptides that are endogenously present in antigenic cells can be used in the invention either alone or in combination with the peptides generated by the proteolytic digestion of the cytosolic and membrane-derived proteins. Peptides that are endogenously present in antigenic cells include peptides that are complexed in vivo to hsp and/or MHC class I and II molecules. According to the invention, such peptides that are isolated directly from a protein preparation of antigenic cells can be complexed to hsps.

In one embodiment, antigenic peptides can be eluted from hsp-peptide complexes either in the presence of ATP or low pH. These experimental conditions can be used to isolate peptides from cells which may contain potentially useful antigenic determinants. Once isolated, the amino acid sequence of each antigenic peptide can be determined using conventional amino acid sequencing methodologies. Such antigenic molecules can then be produced by chemical synthesis or recombinant methods, purified, and complexed to hsps in vitro.

Exemplary, but not limiting, methods that may be used to elute a peptide from a hsp-peptide complex is as follows:

The hsp-peptide complex of interest is centrifuged through a Centricon 10 assembly (Millipore) to remove any low molecular weight material loosely associated with the complex. The large molecular weight fraction may be removed and analyzed by SDS-PAGE while the low molecular weight may be analyzed by HPLC as described below. In one exemplary method, the hsp-peptide complex in the large molecular weight fraction is incubated with 10 mM ATP for 30 minutes at room temperature. In another exemplary method, acetic acid or trifluoroacetic acid (TFA) is added to the stress protein-peptide complex to give a final concentration of 10% (vol/vol) and the mixture incubated at room temperature or in a boiling water bath or any temperature in between, for 10 minutes (See, Van Bleek, et al., 1990, Nature 348:213-216; and Li, et al., 1993, EMBO Journal 12:3143-3151).

The resulting samples are centrifuged through a Centricon 10 assembly as mentioned previously. The high and low molecular weight fractions are recovered. The remaining large molecular weight stress protein-peptide complexes can be reincubated with ATP or low pH to remove any remaining peptides.

The resulting lower molecular weight fractions are pooled, concentrated by evaporation and dissolved in 0.1% TFA. The dissolved material is then fractionated by reverse phase high pressure liquid chromatography (HPLC) using for example a VYDAC C18 reverse phase column equilibrated with 0.1% TFA. The bound material is then eluted at a flow rate of about 0.8 ml/min by developing the column with a linear gradient of 0 to 80% acetonitrile in 0.1% TFA. The elution of the peptides can be monitored by OD210 and the fractions containing the peptides collected.

In another embodiment, antigenic peptides can be isolated from MHC-peptide complexes. The isolation of potentially immunogenic peptides from MHC molecules is well known in the art. See, for example, Falk, et al., 1990, Nature 348:248-251; Rotzsche, et al., 1990, Nature 348:252-254; Elliott, et al., 1990, Nature 348:191-197; Falk, et al., 1991, Nature 351:290-296; Demotz, et al., 1989, Nature 343:682-684; Rotzsche, et al., 1990, Science 249:283-287, the disclosures of which are incorporated herein by reference.

In some embodiments, MHC-peptide complexes can be isolated by a conventional immunoaffinity procedure. The peptides can then be eluted from the MHC-peptide complex by incubating the complexes in the presence of about 0.1% TFA in acetonitrile. The eluted peptides can be fractionated and purified by reverse phase HPLC. The amino acid sequences of the eluted peptides can be determined either by manual or automated amino acid sequencing techniques well known in the art. Once the amino acid sequence of a potentially protective peptide has been determined the peptide can be synthesized in any amount using conventional peptide synthesis or other protocols well known in the art.

Peptides having the same amino acid sequence as those isolated above can be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides can be synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. Commonly used N-α-protecting groups include Boc which is acid labile and Fmoc which is base labile.

In one embodiment, the C-terminal N-α-protected amino acid is first attached to the polystyrene beads. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art (see, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting peptides is accomplished using conventional procedures, such as preparative HPLC using gel permeation, partition and/or ion exchange chromatography. The choice of appropriate matrices and buffers are well known in the art and so are not described in detail herein.

5.12.3 Exogenous Antigenic Molecules

Molecules that display the antigenicity of a known antigen of a pathogen or of a tumor-specific or tumor-associated antigen of a cancer, e.g., antigens or antigenic portions thereof, can be selected for use as antigenic molecules, for complexing to heat shock proteins. Several assays known in the art can be used to determine immunogenicity or antigenicity including, but not limited to, competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are envisioned for use. In one embodiment for detecting immunogenicity, T-cell-mediated responses can be assayed by standard methods, e.g., in vitro cytoxicity assays or in vivo delayed-type hypersensitivity assays.

Potentially useful antigens or derivatives thereof for use as antigenic molecules can also be identified by various criteria, such as the antigen's involvement in neutralization of a pathogen's infectivity (wherein it is desired to treat or prevent infection by such a pathogen) (Norrby, 1985, Summary, in Vaccines 85, Lerner, et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specific for the antigen. In addition, where it is desired to treat or prevent a disease caused by pathogen, the antigen's encoded epitope should preferably display a small or no degree of antigenic variation in time or amongst different isolates of the same pathogen.

5.13 In Vitro Production of HSP/Antigenic Molecule Complexes

In an embodiment in which specific complexes of hsps and the peptides with which they are endogenously associated in vivo are not employed, complexes of hsps and antigenic molecules are produced in vitro. As will be appreciated by those skilled in the art, the peptides either isolated by the aforementioned procedures or chemically synthesized or recombinantly produced may be reconstituted with a variety of purified natural or recombinant stress proteins in vitro to generate immunogenic non-covalent stress protein-antigenic molecule complexes. Alternatively, exogenous antigens or antigenic or immunogenic fragments or derivatives thereof can be complexed to stress proteins for use in the immunotherapeutic or prophylactic vaccines of the invention. A preferred, exemplary protocol for complexing a stress protein and an antigenic molecule in vitro is discussed below.

Prior to complexing, the hsps are pretreated with ATP or low pH to remove any peptides that may be associated with the hsp of interest. When the ATP procedure is used, excess ATP is removed from the preparation by the addition of apyranase as described by Levy, et al., 1991, Cell 67:265-274. When the low pH procedure is used, the buffer is readjusted to neutral pH by the addition of pH modifying reagents.

The antigenic molecules (1 μg) and the pretreated hsp (9 μg) are admixed to give an approximately 5 antigenic molecule: 1 stress protein molar ratio. Then, the mixture is incubated for 15 minutes to 3 hours at 4° to 45° C. in a suitable binding buffer such as one containing 20 mM sodium phosphate, pH 7.2, 350 mM NaCl, 3 mM MgCl₂ and 1 mM phenyl methyl sulfonyl fluoride (PMSF). The preparations are centrifuged through a Centricon 10 assembly (Millipore) to remove any unbound peptide. The association of the peptides with the stress proteins can be assayed by SDS-PAGE. This is the preferred method for in vitro complexing of peptides isolated from MHC-peptide complexes of peptides disassociated from endogenous hsp-peptide complexes.

In an alternative embodiment of the invention, by way of example but not limitation, and preferred for producing complexes of hsp70 to exogenous antigenic molecules such as proteins, 5-10 micrograms of purified hsp is incubated with equimolar quantities of the antigenic molecule in 20 mM sodium phosphate buffer pH 7.5, 0.5M NaCl, 3 mM MgCl₂ and 1 mM ADP in a volume of 100 microliter at 37° C. for 1 hr. This incubation mixture is further diluted to 1 ml in phosphate-buffered saline.

In an alternative embodiment of the invention, by way of example but not limitation, and preferred for producing complexes of gp96 or hsp90 to peptides, 5-10 micrograms of purified gp96 or hsp90 is incubated with equimolar or excess quantities of the antigenic peptide in a suitable buffer such as one containing 20 mM sodium phosphate buffer pH 7.5, 0.5M NaCl, 3 nM MgCl2 at 50-65° C. for 5-20 min. This incubation mixture is allowed to cool to room temperature and centrifuged one or more times if necessary, through a Centricon 10 assembly (Millipore) to remove any unbound peptide.

Following complexing, the immunogenic stress protein-antigenic molecule complexes can optionally be assayed in vitro using for example the mixed lymphocyte target cell assay (MLTC) described below. Once immunogenic complexes have been isolated they can be optionally characterized further in animal models using the preferred administration protocols and excipients discussed below.

5.14 Oligomerization of HSPs and Antigenic Molecules

The oligomerizing agent of the invention can be any compound known in the art for promoting oligomerization of 2 or more hsps, or complexes of hsps and antigenic molecules. Oligomerizing agents that can be used include molecules that promote oligomerization by covalent binding and molecules that promote oligomerization by non-covalent binding to the heat shock proteins or hsp-antigenic molecule complexes. Typically, an oligomerizing agent that promotes oligomerization between 2 or more moieties by covalently binding to them is referred to as a “cross-linker” or a “cross-linking agent.” Cross-linkers are compounds that can react, typically with defined chemical groups, to allow conjugation of two or more moieties to yield oligomers.

Cross-linkers can be bifunctional; i.e., they contain two reactive groups that react with and covalently link two different moieties or two regions in the same moiety, or multi-functiona, e.g., trifunctional. In addition, cross-linkers can be homo-functional (e.g., homobifunctional) or hetero-functional (e.g., heterobifunctional). In homobifunctional cross-linking agents, the reactive groups are identical and these reagents couple like functional groups. In heterobifunctional cross-linking agents, the reactive groups have dissimilar chemistry, allowing the formation of cross-links between unlike functional groups. Several exemplary homobifunctional and heterobifunctional cross-linking agents are discussed below.

As used herein, the term “oligomerizing agent” also includes reagents that promote oligomerization of immunotherapeutic moieties, for example, heat shock proteins, via non-covalent binding. An example of such an oligomerizing agent includes, but is not limited to, bivalent (or bispecific) antibodies. Biotinylation and haptenylation reagents and their cognate binding partners could be considered to be either cross-linking agents or non-covalent oligomerizing agents. These reagents cross-link or covalently bind a biotin or hapten moiety to a target that is to be oligomerized. The biotin or hapten moieties on the covalently modified targets then interact non-covalently with avidin, streptavidin (biotin) or immunoglobulin G (IgG) that can be attached to other target molecules. Avidin, streptavidin, NeutrAvidin biotin-binding protein and CaptAvidin biotin-binding protein can tightly bind up to four molecules of a biotinylated target and IgG can bind up to two haptens.

The present invention provides chemical cross-linking agents for coupling a first heat shock protein molecule to a second heat shock protein molecule, wherein the first and second heat shock protein molecules can, but need not, be identical. In one embodiment, the cross-linking agents are homobifunctional crosslinking agents and typically couple an amine or a thiol group on the first heat shock protein molecule to an amine or a thiol group, respectively, on the second heat shock protein molecule. Homobifunctional crosslinking agents include, for example, homobifunctional amine crosslinkers such as glutaraldehyde, bis(imido esters), bis(succinimidyl esters), diisocyanates and diacid chlorides. Examples of homobifunctional cross-linkers include, but are not limited to, p-Azidophenyl glyoxal monohydrate (APG); 1,8-bis-Maleimidotriethyleneglycol (BM[PEO]₃); 3,3′-Dithiobis[sulfosuccinimidylpropionate]; (DTSSP); Bis[2-(Sulfosuccinimidooxycarbonyloxy)ethyl]sulfone (Sulfo-BSOCOES); Disuccinimidyl suberate (DSS); Ethylene glycol bis[sulfosuccinimidylsuccinate] (Sulfo-EGS); N,N′-bis(3-maleimidpropionyl)-2-hydroxy-1,3-propanediamine; PEG bis(p-nitro-phenyl carbonate) and Dimethyl suberimidate dihydrochloride (DMS). The above, as well as other homobifunctional cross-linkers are available commercially at, e.g., Sigma (St. Louis, Mo.), Pierce Biotechnology, Inc. (Rockford, Ill.) or Molecular Probes (Eugene, Oreg.). Based on the disclosure herein, one of skill in the art will be able to select or design other cross-linkers for use with the invention.

In another embodiment, the cross-linking agent is polyethylene glycol (“PEG”), preferably with derivitization to coupling or activating moieties (e.g., with thiol, triflate, tresylate, aziridune, oxirane, or preferably maleimide). Compounds such as maleimido monomethoxy PEG are exemplary activated PEG compounds. Other examples include, monomethoxy poly(ethylene glycol)-maleimide (mPEG-MAL) or NHS-poly(ethylene glycol)-maleimide (PEG-MAL). The present invention encompasses cross-linking a first and second heat shock protein molecule using a PEG linker.

In another embodiment, the cross-linking agents are heterobifunctional crosslinking agents and typically couple an amine group on the first heat shock protein molecule to a thiol group on the second heat shock protein molecule or vice versa. Examples of heterobifunctional cross-linkers include, but are not limited to, N-succinimidyl-5-acetyl-thioacetate (SATA), N-succinimidyl-3-(2-pyridyldithio)propionate (SPDP), succinimidyl 4-(N-maleimidomethyl) cyclohexane-1-carboxylate (SMCC), sulfosuccinimidyl 4-(N-maleimidomethyl) cyclohexane-1-carboxylate (sSMCC), 2-Iminothiolane (Traut's agent); N-[α-Maleimidoacetoxy]succinimide ester (AMAS); N-1-Maleimidopropionic acid (BMPA); N-[β-Maleimidopropionic acid]hydrazide.TFA (BMPH); 1-Ethyl-3-[3-dimethylaminopropyl]carbodiimide Hydrochloride (EDC); [N-e-Maleimidocaproyloxy]succinimide ester (EMCS); N-[g-Maleimidobutyryloxy]succinimide ester (GMBS); m-Maleimidobenzoyl-N-hydroxysuccinimide ester (MB S) and N-Succinimidyl iodoacetate (SIA). The above, as well as other homobifunctional cross-linkers are available commercially at, e.g., Sigma (St. Louis, Mo.), Pierce Biotechnology, Inc. (Rockford, Ill.) or Molecular Probes (Eugene, Oreg.). Based on the disclosure herein, one of skill in the art will be able to select or design other cross-linkers for use with the invention.

In another embodiment, the cross-linking agents are trifunctional. Trifunctional cross-linkers possess three different reactive or complexing groups per molecule and can oligomeize 3 different biological molecules; e.g., heat shock proteins. Examples of trifunctional cross-linkers include, but are not limited to, β-[Tris(hydroxymethyl) phosphino] propionic acid (THPP) and Tris-succinimidyl aminotriacetate (TSAT). The above, as well as other homobifunctional cross-linkers are available commercially at, e.g., Sigma (St. Louis, Mo.), Pierce Biotechnology, Inc. (Rockford, Ill.) or Molecular Probes (Eugene, Oreg.). Based on the disclosure herein, one of skill in the art will be able to select or design other cross-linkers for use with the invention.

Cross-linking agents of the invention refers to any compound that can promote oligomerization between two or more heat shock protein molecules. In one embodiment, cross-linking agents of the invention are linear or branched substituted aliphatic compounds. In another embodiment, cross-linking agents of the invention are cyclo alkyl, cycloheteroalkyl, substituted cyclo alkyl or substituted cycloheteroalkyl. In one embodiment, the cross-linking agent is photoreactive.

In one embodiment, the oligomerizing agent of the invention is a streptavidin-biotin linker. In this embodiment, the biotin can be conjugated with a first heat shock protein while avidin or streptavidin can be conjugated with a second heat shock protein. The strong affinity of biotin for avidin and streptavidin results in a stable biotin-avidin or biotin-streptavidin bond that in turn can promote oligomerization of the heat shock protein molecules conjugated to the biotin and avidin/streptavidin. In some embodiments, the first and second heat shock protein molecules are identical.

In another embodiment, the oligomerizing agent is a multivalent, for example, a bivalent (also called bispecific) antibody. A bispecific antibody binds non-covalently to a first and a second protein, e.g., a heat shock protein, for which the antibody is specific, thereby promoting oligomerization of the first and second protein. In some embodiments, the first and second proteins are identical.

According to the invention, oligomerizing agents are used that do not destroy the immunomodulatory activity of the heat shock protein moieties, e.g., the hsp-peptide cmplexes. In one embodiment, the oligomerizing agent does not disrupt the binding of antigenic molecules, e.g., peptides, to the heat shock proteins. In another embodiment, the oligomerizing agent does not disrupt the binding of the hsp to its receptor. In another embodiment, the oligomerizing agent is active and functional at neutral pH.

Based on the above disclosure, other oligomerizing agents for promoting oligomerization of heat shock proteins will be apparent to one of skill in the art. The above disclosed, as well as other cross-linkers are available commercially from several companies, such as Sigma (St. Louis, Mo.; see Sigma catalog, 2002-2003, pages 573-580), Pierce Biotechnology, Inc. (Rockford, Ill.; see Pierce Biotechnology, Inc. catalog, 2001-2002, pages 294-334) or Molecular Probes (Eugene, Oreg.; see Molecular Probes Handbook, 9^{th} ed., 2002, chapter 5), each of which is incorporated herein by reference in its entirety.

In specific embodiments of the invention, certain compounds are not used as oligomerizing agents. In one embodiment, an oligomerizing agent of the invention is not a lectin. In another embodiment, an oligomerizing agent of the invention is a lectin. In another embodiment, an oligomerizing agent of the invention is not glutaraldehyde. In another embodiment, an oligomerizing agent of the invention is not sulfosuccinimidyl (4-azidosalicylamido) hexanoate (“SASD”). In yet another embodiment, an oligomerizing agent of the invention is not a member of the BAG family proteins (see Takayama and Reed, 2001, Nature Cell Biology 3:E237-E241).

In another embodiment, the oligomerizing agent of the invention is not 4,4′-dianilino-1,1′-binaphthyl-5,5′-disulfonic acid (“bis-ANS”), 1,8-anilinonaphthalene sulfonate (“8-ANS”) or N-ethylcarboxamidoadenosine (“NECA”). In another embodiment, an oligomerizing agent of the invention is not a compound comprising an adenosine moiety or a structural mimetic thereof. Structural mimetics of an adenosine moiety include those molecules that have any variety of substitutions at the 2′, 3′ and 5′ positions of adenosine (see Gewirth et al., U.S. Patent Application Publication No. 2002/0160496).

5.15 Determination of Immunogenicity of HSP-Peptide Complexes

Optionally, the specific complexes and the diluted complexes of the invention can be assayed for immunogenicity using any method known in the art. By way of example but not limitation, one of the following procedures can be used.

5.15.1 The MLTC Assay

Briefly, mice are injected with an amount of the specific or diluted hsp-peptide complex, using any convenient route of administration. As a negative control, other mice are injected with, e.g., hsp-peptide complexes that are to be used as non-specific or diluent complexes. Cells known to contain specific antigens, e.g. tumor cells or cells infected with an agent of an infectious disease, may act as a positive control for the assay. The mice are injected twice, 7-10 days apart. Ten days after the last immunization, the spleens are removed and the lymphocytes released. The released lymphocytes may be re-stimulated subsequently in vitro by the addition of dead cells that expressed the antigen of interest.

For example, 8×10⁶ immune spleen cells may be stimulated with 4×10⁴ mitomycin C treated or γ-irradiated (5-10,000 rads) cells containing the antigen of interest (or cells transfected with an appropriate gene, as the case may be) in 3 ml RPMI medium containing 10% fetal calf serum. In certain cases 33% secondary mixed lymphocyte culture supernatant may be included in the culture medium as a source of T-cell growth factors (See, Glasebrook, et al., 1980, J. Exp. Med. 151:876). To test the primary cytotoxic T-cell response after immunization, spleen cells may be cultured without stimulation. In some experiments spleen cells of the immunized mice may also be re-stimulated with antigenically distinct cells, to determine the specificity of the cytotoxic T-cell response.

Six days later the cultures are tested for cytotoxicity in a 4 hour ⁵¹Cr-release assay (See, Palladino, et al., 1987, Cancer Res. 47:5074-5079 and Blachere, at al., 1993, J. Immunotherapy 14:352-356). In this assay, the mixed lymphocyte culture is added to a target cell suspension to give different effector:target (E:T) ratios (usually 1:1 to 40:1). The target cells are prelabelled by incubating 1×10⁶ target cells in culture medium containing 20 mCi ⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelletted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

5.15.2 CD4⁺ T-Cell Proliferation Assay

Primary T-cells are obtained from spleen, fresh blood, or CSF and purified by centrifugation using FICOLL-PAQUE PLUS (Pharmacia, Upsalla, Sweden) essentially as described by Kruse and Sebald, 1992, EMBO J. 11: 3237-3244. The peripheral blood mononuclear cells are incubated for 7-10 days with a lysate of cells expressing an antigenic molecule. Antigen presenting cells may, optionally be added to the culture 24 to 48 hours prior to the assay, in order to process and present the antigen in the lysate. The cells are then harvested by centrifugation, and washed in RPMI 1640 media (GibcoBRL, Gaithersburg, Md.). 5×10⁴ activated T-cells/well (PHA-blasts) are in RPMI 1640 media containing 10% fetal bovine serum, 10 mM HEPES, pH 7.5, 2 mM L-glutamine, 100 units/ml penicillin G, and 100 μg/ml streptomycin sulphate in 96 well plates for 72 hrs at 37° C., pulsed with 1 μCi ³H-thymidine (DuPont NEN, Boston, Mass.)/well for 6 hrs, harvested, and radioactivity measured in a TOPCOUNT scintillation counter (Packard Instrument Co., Meriden, Conn.).

5.15.3 Antibody Response Assay

In a certain embodiment of the invention, the immunogenicity of an hsp-peptide complex is determined by measuring antibodies produced in response to the vaccination with the complex. In one mode of the embodiment, microtitre plates (96-well Immuno Plate II, Nunc) are coated with 50 μl/well of a 0.75 μg/ml solution of a purified, non-hsp-complexed form of the peptide used in the vaccine (e.g. Aβ42) in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or CSF from a vaccinated animal (such as a model mouse or a human patient) is applied at 20° C. for 1 hour, and the plates are washed 3 times with PBS-T. The anti-peptide antibody activity is then measured calorimetrically after incubating at 20° C. for 1 hour with 50 μl/well of sheep anti-mouse or anti-human immunoglobulin, as appropriate, conjugated with horseradish peroxidase (Amersham) diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a Kontron SLT-210 photometer (SLT Lab-instr., Zurich, Switzerland) at 492 nm (ref. 620 nm).

5.15.4 Cytokine Detection Assay

The CD4⁺ T-cell proliferative response to hsp-peptide complexes of the invention may be measured by detection and quantitation of the levels of specific cytokines. In one embodiment, for example, intracellular cytokines may be measured using an IFN-γ detection assay to test for immunogenicity of a complex of the invention. In an example of this method, peripheral blood mononuclear cells from a subject treated with a hsp-peptide complex are stimulated with peptide antigens of a given tumor or with peptide antigens of an agent of infectious disease. Cells are then stained with T-cell-specific labeled antibodies detectable by flow cytometry, for example FITC-conjugated anti-CD8 and PerCP-labeled anti-CD4 antibodies. After washing, cells are fixed, permeabilized, and reacted with dye-labeled antibodies reactive with human IFN-γ (PE-anti-IFN-γ). Samples are analyzed by flow cytometry using standard techniques.

Alternatively, a filter immunoassay, the enzyme-linked immunospot assay (ELISPOT) assay, may be used to detect specific cytokines surrounding a T-cell. In one embodiment, for example, a nitrocellulose-backed microtiter plate is coated with a purified cytokine-specific primary antibody, i.e., anti-IFN-γ, and the plate is blocked to avoid background due to nonspecific binding of other proteins. A sample of mononuclear blood cells containing cytokine-secreting cells obtained from a subject treated with a hsp-peptide complex is diluted onto the wells of the microtitre plate. A labeled, e.g., biotin-labeled, secondary anti-cytokine antibody is added. The antibody cytokine complex can then be detected. The enzyme-conjugated streptavidin—cytokine-secreting cells will appear as “spots” by visual, microscopic, or electronic detection methods.

5.15.5 Tetramer Assay

In another embodiment, the “tetramer staining” assay (Altman et al., 1996, Science 274: 94-96) can be used to identify antigen-specific T-cells. For example, in one embodiment, an MHC molecule containing a specific peptide antigen, such as a tumor-specific antigen, can be multimerized to make soluble peptide tetramers and labeled, for example, by complexing to streptavidin. The MHC-peptide antigen complex is then mixed with a population of T-cells obtained from a subject treated with a hsp-peptide complex. Biotin can be used to stain T-cells which express the antigen of interest, i.e., the tumor-specific antigen.

5.16 Combination with Adoptive Immunotherapy

Adoptive immunotherapy refers to a therapeutic approach for treating cancer or infectious diseases in which immune cells are administered to a host with the aim that the cells mediate either directly or indirectly specific immunity to tumor cells and/or antigenic components or regression of the tumor or treatment of infectious diseases, as the case may be. (See U.S. Pat. No. 5,985,270, issued Nov. 16, 1999, which is incorporated by reference herein in its entirety.) As an optional step, in accordance with the methods described herein, APC are sensitized with hsps complexed with antigenic (or immunogenic) molecules and used in adoptive immunotherapy.

In a specific embodiment, therapy by administration of diluted complexes, using any route of administration, may optionally be combined with adoptive immunotherapy using APC sensitized with hsp-antigenic molecule complexes. The hsp-peptide complex-sensitized APC can be administered alone, in combination with the diluted hsp-peptide complexes, or before or after administration of the diluted hsp-peptide complexes. Furthermore, the mode of administration can be varied, including but not limited to, e.g., subcutaneously, intravenously or intramuscularly, although intradermally or mucosally is preferred.

5.16.1 Obtaining Macrophages and Antigen Presenting Cells

The antigen-presenting cells, including but not limited to macrophages, dendritic cells and B-cells, are preferably obtained by production in vitro from stem and progenitor cells from human peripheral blood or bone marrow as described by Inaba, K., et al., 1992, J. Exp. Med., 176:1693-1702.

APC can be obtained by any of various methods known in the art. In a preferred aspect human macrophages are used, obtained from human blood cells. By way of example but not limitation, macrophages can be obtained as follows:

Mononuclear cells are isolated from peripheral blood of a patient (preferably the patient to be treated), by Ficoll-Hypaque gradient centrifugation and are seeded on tissue culture dishes which are pre-coated with the patient's own serum or with other AB+ human serum. The cells are incubated at 37° C. for 1 hour, then non-adherent cells are removed by pipetting. To the adherent cells left in the dish, is added cold (4° C.) 1 mM EDTA in phosphate-buffered saline and the dishes are left at room temperature for 15 minutes. The cells are harvested, washed with RPMI buffer and suspended in RPMI buffer. Increased numbers of macrophages may be obtained by incubating at 37° C. with macrophage-colony stimulating factor (M-CSF); increased numbers of dendritic cells may be obtained by incubating with granulocyte-macrophage-colony stimulating factor (GM-CSF) as described in detail by Inaba, K., et al., 1992, J. Exp. Med., 176:1693-1702.

5.16.2 Sensitizing Macrophages and APCs with Hsp-Peptide Complexes

APCs can be sensitized with hsp bound to antigenic molecules preferably by incubating the cells in vitro with the complexes. The APC are sensitized with complexes of hsps and antigenic molecules by incubating in vitro with the hsp-complex at 37° C. for 15 minutes to 24 hours. By way of example but not limitation, 4×10⁷ macrophages can be incubated with 10 microgram gp96-peptide complexes per ml or 100 microgram hsp90-peptide complexes per ml at 37° C. for 15 minutes-24 hours in 1 ml plain RPMI medium. The cells are washed three times and resuspended in a physiological medium preferably sterile, at a convenient concentration (e.g., 1×10⁷/ml) for injection in a patient. Preferably, the patient into which the sensitized APCs are injected is the patient from which the APC were originally isolated (autologous embodiment).

Optionally, the ability of sensitized APCs to stimulate, for example, the antigen-specific, class I-restricted cytotoxic T-lymphocytes (CTL) can be monitored by their ability to stimulate CTLs to release tumor necrosis factor, and by their ability to act as targets of such CTLs.

5.16.3 Reinfusion of Sensitized APC

The hsp-antigenic molecule-sensitized APCs are reinfused into the patient systemically, preferably intravenously, by conventional clinical procedures. These activated cells are reinfused, preferentially by systemic administration into the autologous patient. Patients generally receive from about 10⁶ to about 10¹² sensitized macrophages, depending on the condition of the patient. In some regimens, patients may optionally receive in addition a suitable dosage of a biological response modifier including but not limited to the cytokines IFN-α, IFN-γ, IL-2, IL-4, IL-6, TNF or other cytokine growth factor.

5.17 Passive Immunotherapy

The compositions of the invention can also be used for passive immunotherapy against cancers and infectious diseases. Passive immunity is the short-term protection of a host, achieved by the administration of pre-formed antibody directed against a heterologous organism. For example, compositions of the invention comprising diluted hsp-peptide complexes obtained from cells infected with an infectious organism may be used to elicit an immune response in a subject, the sera removed from the subject and used for treatment or prevention of a disease caused the infectious organism in another subject.

5.18 Prevention and Treatment of Diseases

In accordance with the invention, a composition of the invention, which comprises complexes of antigenic peptides, e.g. peptides derived from digested cytosolic and/or membrane-derived proteins of antigenic cells or viral particle, and a hsp, is administered to a subject with cancer or an infectious disease. In one embodiment, “treatment” or “treating” refers to an amelioration of cancer or an infectious disease, or at least one discernible symptom thereof. In another embodiment, “treatment” or “treating” refers to an amelioration of at least one measurable physical parameter associated with cancer or an infectious disease, not necessarily discernible by the subject. In yet another embodiment, “treatment” or “treating” refers to inhibiting the progression of a cancer or an infectious disease, either physically, e.g., stabilization of a discernible symptom, or physiologically, e.g., stabilization of a physical parameter, or both.

In certain embodiments, the compositions of the present invention are administered to a subject as a preventative measure against such cancer or an infectious disease. As used herein, “prevention” or “preventing” refers to a reduction of the risk of acquiring a given cancer or infectious disease. In one mode of the embodiment, the compositions of the present invention are administered as a preventative measure to a subject having a genetic predisposition to a cancer. In another mode of the embodiment, the compositions of the present invention are administered as a preventive measure to a subject facing exposure to carcinogens including but not limited to chemicals, radiation, cigarette smoke, or combinations thereof, or to a subject facing exposure to an agent of an infectious disease. In a specific embodiment, the compositions of the present invention are administered as a preventative measure to a subject environmentally exposed to carcinogens, for example cigarette smoke.

For example, in certain embodiments, administration of the compositions of the invention leads to an inhibition or reduction of the growth of cancerous cells or infectious agents by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth in absence of said composition.

The compositions prepared by methods of the invention comprise complexes of heat shock protein(s) with a population of antigenic peptides. The compositions appear to induce an inflammatory reaction at the tumor site and can ultimately cause a regression of the tumor burden in the cancer patients treated. The compositions prepared by the methods of the invention can enhance the immunocompetence of the subject and elicit specific immunity against infectious agents or specific immunity against preneoplastic and neoplastic cells. These compositions have the capacity to prevent the onset and progression of infectious diseases, and to inhibit the growth and progression of tumor cells.

Combination therapy refers to the use of hsp-peptide complexes or compositions of the invention with another modality to prevent or treat cancer and infectious diseases. The administration of the complexes of the invention can augment the effect of anti-cancer agents or anti-infectives, and vice versa. Preferably, this additional form of modality is a non-hsp based modality, i.e., this modality does not comprise heat shock proteins as a component. This approach is commonly termed combination therapy, adjunctive therapy or conjunctive therapy (the terms are used interchangeably herein). With combination therapy, additive potency or additive therapeutic effect can be observed. Synergistic outcomes where the therapeutic efficacy is greater than additive can also be expected. The use of combination therapy can also provide better therapeutic profiles than the administration of either the treatment modality, or the hsp-peptide complexes alone. The additive or synergistic effect may allow the dosage and/or dosing frequency of either or both modalities be adjusted to reduce or avoid unwanted or adverse effects.

In various specific embodiments, the combination therapy comprises the administration of hsp-peptide complexes to a subject treated with a treatment modality wherein the treatment modality administered alone is not clinically adequate to treat the subject such that the subject needs additional effective therapy, e.g., a subject is unresponsive to a treatment modality without administering hsp-peptide complexes. Included in such embodiments are methods comprising administering hsp-peptide complexes to a subject receiving a treatment modality wherein said subject has responded to therapy yet suffers from side effects, relapse, develops resistance, etc. Such a subject might be non-responsive or refractory to treatment with the treatment modality alone, i.e., at least some significant portion of cancer cells or pathogens are not killed or their cell division is not arrested. The embodiments provide that the methods of the invention comprising administration of hsp-peptide complexes to a subject refractory to a treatment modality alone can improve the therapeutic effectiveness of the treatment modality when administered as contemplated by the methods of the invention. The determination of the effectiveness of a treatment modality can be assayed in vivo or in vitro using methods known in the art. Art-accepted meanings of refractory are well known in the context of cancer. In one embodiment, a cancer or infectious disease is refractory or non-responsive where respectively, the number of cancer cells or pathogens has not been significantly reduced, or has increased. Among these subjects being treated are those receiving chemotherapy or radiation therapy.

According to the invention, complexes of the invention can be used in combination with many different types of treatment modalities. Some of such modalities are particularly useful for a specific type of cancer or infectious disease. Many other modalities have an effect on the functioning of the immune system and are applicable generally to both neoplastic and infectious diseases.

In one embodiment, complexes of the invention are used in combination with one or more biological response modifiers to treat cancer or infectious disease. One group of biological response modifiers is the cytokines. In one such embodiment, a cytokine is administered to a subject receiving hsp-peptide complexes. In another such embodiment, hsp complexes are administered to a subject receiving a chemotherapeutic agent in combination with a cytokine. In various embodiments, one or more cytokine(s) can be used and are selected from the group consisting of IL-1α, IL-1β, IL-2, IL-3, IL-4, IL-5, IL-6, IL-7, IL-8, IL-9, IL-10, IL-11, IL-12, IFNα, IFNβ, IFNγ, TNFα, TNF, G-CSF, GM-CSF, TGF-β, IL-15, IL-18, GM-CSF, INF-γ, INF-α, SLC, endothelial monocyte activating protein-2 (EMAP2), MIP-3α, MIP-3β, or an MHC gene, such as HLA-B7. Addtionally, other exemplary cytokines include other members of the TNF family, including but not limited to TNF-α-related apoptosis-inducing ligand (TRAIL), TNF-α-related activation-induced cytokine (TRANCE), TNF-α-related weak inducer of apoptosis (TWEAK), CD40 ligand (CD40L), lymphotoxin alpha (LT-α), lymphotoxin beta (LT-β), OX40 ligand (OX40L), Fas ligand (FasL), CD27 ligand (CD27L), CD30 ligand (CD30L), 41BB ligand (41BBL), APRIL, LIGHT, TL1, TNFSF16, TNFSF17, and AITR-L, or a functional portion thereof. See, e.g., Kwon et al., 1999, Curr. Opin. Immunol. 11:340-345 for a general review of the TNF family. Preferably, the hsp-peptide complex is administered prior to the treatment modalities. In a specific embodiment, one or more complexes of the invention are administered to a subject receiving cyclophosphamide in combination with IL-12 for treatment of cancer.

In another embodiment, complexes of the invention are used in combination with one or more biological response modifiers which are agonists or antagonists of various ligands, receptors and signal transduction molecules of the immune system. For examples, the biological response modifiers include but are not limited to agonists of Toll-like receptors (TLR-2, TLR-7, TLR-8 and TLR-9; LPS; agonists of 41BB ligand, OX40 ligand, ICOS, and CD40; and antagonists of Fas ligand, PD1, and CTLA4. These agonists and antagonists can be antibodies, antibody fragments, peptides, peptidomimetic compounds, and polysaccharides.

In yet another embodiment, complexes of the invention are used in combination with one or more biological response modifiers which are immunostimulatory nucleic acids. Such nucleic acids, many of which are oligonucleotides comprising an unmethylated CpG motif, are mitogenic to vertebrate lymphocytes, and are known to enhance the immune response. See Woolridge, et al., 1997, Blood 89:2994-2998. Such oligonucleotides are described in International Patent Publication Nos. WO 01/22972, WO 01/51083, WO 98/40100 and WO 99/61056, each of which is incorporated herein in its entirety, as well as U.S. Pat. Nos. 6,207,646, 6,194,388, 6,218,371, 6,239,116, 6,429,199, and 6,406,705, each of which is incorporated herein in its entirety. Other kinds of immunostimulatory oligonucleotides such as phosphorothioate oligodeoxynucleotides containing YpG- and CpR-motifs have been described by Kandimalla et al. in “Effect of Chemical Modifications of Cytosine and Guanine in a CpG-Motif of Oligonucleotides: Structure-Immunostimulatory Activity Relationships.” Bioorganic & Medicinal Chemistry 9:807-813 (2001), incorporated herein by reference in its entirety. Also encompassed are immunostimulatory oligonucleotides that lack CpG dinucleotides which when administered by mucosal routes (including low dose administration) or at high doses through parenteral routes, augment antibody responses, often as much as did the CpG nucleic acids, however the response was Th2-biased (IgG1>>IgG2a). See U.S. Patent Publication No. 20010044416 A1, which is incorporated herein by reference in its entirety. Methods of determining the activity of immunostimulatory oligonucleotides can be performed as described in the aforementioned patents and publications. Moreover, immunostimulatory oligonucleotides can be modified within the phosphate backbone, sugar, nucleobase and internucleotide linkages in order to modulate the activity. Such modifications are known to those of skill in the art.

In yet another embodiment, complexes of the invention are used in combination with one or more adjuvants. The adjuvant(s) can be administered separately or present in a composition in admixture with complexes of the invention. A systemic adjuvant is an adjuvant that can be delivered parenterally. Systemic adjuvants include adjuvants that creates a depot effect, adjuvants that stimulate the immune system and adjuvants that do both. An adjuvant that creates a depot effect as used herein is an adjuvant that causes the antigen to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

Other adjuvants stimulate the immune system, for instance, cause an immune cell to produce and secrete cytokines or IgG. This class of adjuvants includes but is not limited to immunostimulatory nucleic acids, such as CpG oligonucleotides; saponins purified from the bark of the Q. saponaria tree, such as QS21; poly[di(carboxylatophen-oxy)phosphazene (PCPP polymer; Virus Research Institute, USA); derivatives of lipopolysaccharides (LPS) such as monophosphoryl lipid A (MPL; Ribi ImmunoChem Research, Inc., Hamilton, Mont.), muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.), aminoalkyl glucosaminide phosphates (AGP; Corixa Corporation, Seattle, Wash.).

Other systemic adjuvants are adjuvants that create a depot effect and stimulate the immune system. These compounds are those compounds which have both of the above-identified functions of systemic adjuvants. This class of adjuvants includes but is not limited to ISCOMs (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The mucosal adjuvants useful according to the invention are adjuvants that are capable of inducing a mucosal immune response in a subject when administered to a mucosal surface in conjunction with complexes of the invention. Mucosal adjuvants include but are not limited to CpG nucleic acids (e.g. PCT published patent application WO 99/61056), Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192 g (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); bacterial outer membrane proteins (e.g., outer surface protein A (OspA) lipoprotein of Borrelia burgdorferi, outer membrane protine of Neisseria meningitidis)(Marinaro et al., 1999, Van de Verg et al., 1996); oil-in-water emulsions (e.g., MF59) (Barchfield et al., 1999, Verschoor et al., 1999, O'Hagan, 1998); aluminum salts (Isaka et al., 1998, 1999); and Saponins (e.g., QS21) Aquila Biopharmaceuticals, Inc., Worster, Me.) (Sasaki et al., 1998, MacNeal et al., 1998), ISCOMs, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

5.18.1 Treatment of Cancers

In one embodiment, combination therapy encompasses, in addition to the administration of the complexes of the invention, the adjunctive uses of one or more modalities that aid in the prevention or treatment of cancer. Examples of modalities include, but are not limited to, chemotherapeutic agents, immunotherapeutics, anti-angiogenic agents, cytokines, hormones, antibodies, polynucleotides, radiation and photodynamic therapeutic agents. In specific embodiments, combination therapy can be used to prevent the recurrence of cancer, inhibit metastasis, or inhibit the growth and/or spread of cancer or metastasis.

Types of cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease.

In another embodiment, a patient having a cancer is immunosuppressed by reason of having undergone anti-cancer therapy (e.g., chemotherapy radiation) prior to administration of the hsp-peptide complexes or administration of the hsp-sensitized APC.

There are many reasons why immunotherapy as provided by the present invention is desired for use in cancer patients. First, surgery with anesthesia may lead to immunosuppression. With appropriate immunotherapy in the preoperative period, this immunosuppression may be prevented or reversed. This could lead to fewer infectious complications and to accelerated wound healing. Second, tumor bulk is minimal following surgery and immunotherapy is most likely to be effective in this situation. A third reason is the possibility that tumor cells are shed into the circulation at surgery and effective immunotherapy applied at this time can eliminate these cells.

The preventive and therapeutic methods of the invention are directed at enhancing the immunocompetence of the cancer patient either before, during, or after surgery, and to induce tumor-specific immunity to cancer cells, with the objective being inhibition of cancer, and with the ultimate clinical objective being total cancer regression and eradication. The methods of the invention can also be used in individuals with enhanced risk of a particular type of cancer, e.g., due to familial history or environmental risk factors.

In various embodiments, one or more anti-cancer agent, in addition to the complexes of the invention, are administered to treat a cancer patient. An anti-cancer agent refers to any molecule or compound that assists in the treatment of tumors or cancers. Examples of anti-cancer agents that can be used in the methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs that can be used include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacteriun cell wall sk; mopidamol; multiple drug resistance gene inhibitor; multiple tumor suppressor 1-based therapy; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

An anti-cancer agent can be a chemotherapeutic agents which include but are not limited to, the following groups of compounds: cytotoxic antibiotics, antimetabolities, anti-mitotic agents, alkylating agents, platinum compounds, arsenic compounds, DNA topoisomerase inhibitors, taxanes, nucleoside analogues, plant alkaloids, and toxins; and synthetic derivatives thereof. Table 1 lists exemplary compounds of the groups:

TABLE 1

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| Platinum containing compounds: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxol |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| mitomycins: | Mitomycin C |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Floxuridine |
| | Doxifluridine |
| | Ratitrexed |
| Cytosine analogs: | Cytarabine (ara C) |
| | Cytosine arabinoside |
| | Fludarabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | pyrazoloimidazole |
| Antimitotic agents: | allocolchicine |
| | Halichondrin B |
| | colchicine |
| | colchicine derivative |
| | dolstatin 10 |
| | maytansine |
| | rhizoxin |
| | thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ATPase inhibitors: | Thapsigargin |

Compositions comprising one or more chemotherapeutic agents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone. Each of the foregoing lists is illustrative, and is not intended to be limiting.

In one embodiment, breast cancer can be treated with a pharmaceutical composition comprising complexes of the invention in combination with 5-fluorouracil, cisplatin, docetaxel, doxorubicin, HERCEPTIN®, gemcitabine, IL-2, paclitaxel, and/or VP-16 (etoposide).

In another embodiment, prostate cancer can be treated with a pharmaceutical composition comprising complexes of the invention in combination with paclitaxel, docetaxel, mitoxantrone, and/or an androgen receptor antagonist (e.g., flutamide).

In another embodiment, leukemia can be treated with a pharmaceutical composition comprising complexes of the invention in combination with fludarabine, cytosine arabinoside, gemtuzumab (MYLOTARG), daunorubicin, methotrexate, vincristine, 6-mercaptopurine, idarubicin, mitoxantrone, etoposide, asparaginase, prednisone and/or cyclophosphamide. As another example, myeloma can be treated with a pharmaceutical composition comprising complexes of the invention in combination with dexamethasone. Preferably, the leukemia is chronic myeloid leukemia (CML), the Hsp-peptide complexes comprises hsp70-peptide complexes, and the therapeutic modality is imatinib mesylate or GLEEVEC™.

In another embodiment, melanoma can be treated with a pharmaceutical composition comprising complexes of the invention in combination with dacarbazine.

In another embodiment, colorectal cancer can be treated with a pharmaceutical composition comprising complexes of the invention in combination with irinotecan.

In another embodiment, lung cancer can be treated with a pharmaceutical composition comprising complexes of the invention in combination with paclitaxel, docetaxel, etoposide and/or cisplatin.

In another embodiment, non-Hodgkin's lymphoma can be treated with a pharmaceutical composition comprising complexes of the invention in combination with cyclophosphamide, CHOP, etoposide, bleomycin, mitoxantrone and/or cisplatin.

In another embodiment, gastric cancer can be treated with a pharmaceutical composition comprising complexes of the invention in combination with cisplatin.

In another embodiment, pancreatic cancer can be treated with a pharmaceutical composition comprising complexes of the invention in combination with gemcitabine.

According to the invention, the complexes of the invention can be administered prior to, subsequently, or concurrently with anti-cancer agent(s), for the prevention or treatment of cancer. Depending on the type of cancer, the subject's history and condition, and the anti-cancer agent(s) of choice, the use of the complexes of the invention can be coordinated with the dosage and timing of chemotherapy.

The use of the complexes of the invention can be added to a regimen of chemotherapy. In one embodiment, the chemotherapeutic agent is gemcitabine at a dose ranging from 100 to 1000 mg/m²/cycle. In one embodiment, the chemotherapeutic agent is dacarbazine at a dose ranging from 200 to 4000 mg/m²/cycle. In a preferred embodiment, the dose of dacarbazine ranges from 700 to 1000 mg/m²/cycle. In another embodiment, the chemotherapeutic agent is fludarabine at a dose ranging from 25 to 50 mg/m²/cycle. In another embodiment, the chemotherapeutic agent is cytosine arabinoside (Ara-C) at a dose ranging from 200 to 2000 mg/m²/cycle. In another embodiment, the chemotherapeutic agent is docetaxel at a dose ranging from 1.5 to 7.5 mg/kg/cycle. In another embodiment, the chemotherapeutic agent is paclitaxel at a dose ranging from 5 to 15 mg/kg/cycle. In yet another embodiment, the chemotherapeutic agent is cisplatin at a dose ranging from 5 to 20 mg/kg/cycle. In yet another embodiment, the chemotherapeutic agent is 5-fluorouracil at a dose ranging from 5 to 20 mg/kg/cycle. In yet another embodiment, the chemotherapeutic agent is doxorubicin at a dose ranging from 2 to 8 mg/kg/cycle. In yet another embodiment, the chemotherapeutic agent is epipodophyllotoxin at a dose ranging from 40 to 160 mg/kg/cycle. In yet another embodiment, the chemotherapeutic agent is cyclophosphamide at a dose ranging from 50 to 200 mg/kg/cycle. In yet another embodiment, the chemotherapeutic agent is irinotecan at a dose ranging from 50 to 75, 75 to 100, 100 to 125, or 125 to 150 mg/m²/cycle. In yet another embodiment, the chemotherapeutic agent is vinblastine at a dose ranging from 3.7 to 5.4, 5.5 to 7.4, 7.5 to 11, or 11 to 18.5 mg/m²/cycle. In yet another embodiment, the chemotherapeutic agent is vincristine at a dose ranging from 0.7 to 1.4, or 1.5 to 2 mg/m²/cycle. In yet another embodiment, the chemotherapeutic agent is methotrexate at a dose ranging from 3.3 to 5, 5 to 10, 10 to 100, or 100 to 1000 mg/m²/cycle.

In a preferred embodiment, the invention further encompasses the use of low doses of chemotherapeutic agents when administered as part of the combination therapy regimen. For example, initial treatment with the complexes of the invention increases the sensitivity of a tumor to subsequent challenge with a dose of chemotherapeutic agent, which dose is near or below the lower range of dosages when the chemotherapeutic agent is administered without complexes of the invention.

In one embodiment, complexes of the invention and a low dose (e.g., 6 to 60 mg/m²/day or less) of docetaxel are administered to a cancer patient. In another embodiment, complexes of the invention and a low dose (e.g., 10 to 135 mg/m²/day or less) of paclitaxel are administered to a cancer patient. In yet another embodiment, complexes of the invention and a low dose (e.g., 2.5 to 25 mg/m²/day or less) of fludarabine are administered to a cancer patient. In yet another embodiment, complexes of the invention and a low dose (e.g., 0.5 to 1.5 g/m²/day or less) of cytosine arabinoside (Ara-C) are administered to a cancer patient. In another embodiment, the chemotherapeutic agent is gemcitabine at a dose ranging from 10 to 100 mg/m²/cycle. In another embodiment, the chemotherapeutic agent is cisplatin, e.g., PLATINOL or PLATINOL-AQ (Bristol Myers), at a dose ranging from 5 to 10, 10 to 20, 20 to 40, or 40 to 75 mg/m²/cycle. In yet another embodiment, a dose of cisplatin ranging from 7.5 to 75 mg/m²/cycle is administered to a patient with ovarian cancer. In yet another embodiment, a dose of cisplatin ranging from 5 to 50 mg/m²/cycle is administered to a patient with bladder cancer. In yet another embodiment, the chemotherapeutic agent is carboplatin, e.g., PARAPLATIN (Bristol Myers), at a dose ranging from 2 to 4, 4 to 8, 8 to 16, 16 to 35, or 35 to 75 mg/m²/cycle. In yet another embodiment, a dose of carboplatin ranging from 7.5 to 75 mg/m²/cycle is administered to a patient with ovarian cancer. In another embodiment, a dose of carboplatin ranging from 5 to 50 mg/m²/cycle is administered to a patient with bladder cancer. In yet another embodiment, a dose of carboplatin ranging from 2 to 20 mg/m²/cycle is administered to a patient with testicular cancer. In yet another embodiment, the chemotherapeutic agent is docetaxel, e.g., TAXOTERE (Rhone Poulenc Rorer) at a dose ranging from 6 to 10, 10 to 30, or 30 to 60 mg/m²/cycle. In yet another embodiment, the chemotherapeutic agent is paclitaxel, e.g., TAXOL (Bristol Myers Squibb), at a dose ranging from 10 to 20, 20 to 40, 40 to 70, or 70 to 135 mg/kg/cycle. In yet another embodiment, the chemotherapeutic agent is 5-fluorouracil at a dose ranging from 0.5 to 5 mg/kg/cycle. In yet another embodiment, the chemotherapeutic agent is doxorubicin, e.g., ADRLAMYCIN (Pharmacia & Upjohn), DOXIL (Alza), RUBEX (Bristol Myers Squibb), at a dose ranging from 2 to 4, 4 to 8, 8 to 15, 15 to 30, or 30 to 60 mg/kg/cycle.

In another embodiment, complexes of the invention are administered in combination with one or more immunotherapeutic agents, such as antibodies and vaccines. In a preferred embodiment, the antibodies have in vivo therapeutic and/or prophylactic uses against cancer. In some embodiments, the antibodies can be used for treatment and/or prevention of infectious disease. Examples of therapeutic and prophylactic antibodies include, but are not limited to, MDX-010 (Medarex, NJ) which is a humanized anti-CTLA4 antibody currently in clinic for the treatment of prostate cancer; SYNAGIS® (Medimmune, MD) which is a humanized anti-respiratory syncytial virus (RSV) monoclonal antibody for the treatment of patients with RSV infection; HERCEPTIN® (Trastuzumab) (Genentech, CA) which is a humanized anti-HER2 monoclonal antibody for the treatment of patients with metastatic breast cancer. Other examples are a humanized anti-CD18 F(ab′)₂ (Genentech); CDP860 which is a humanized anti-CD18 F(ab′)₂ (Celltech, UK); PRO542 which is an anti-HIV gp120 antibody fused with CD4 (Progenics/Genzyme Transgenics); Ostavir which is a human anti Hepatitis B virus antibody (Protein Design Lab/Novartis); PROTOVIR™ which is a humanized anti-CMV IgG1 antibody (Protein Design Lab/Novartis); MAK-195 (SEGARD) which is a murine anti-TNF-α F(ab′)₂ (Knoll Pharma/BASF); IC14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); BEC2 which is a murine anti-idiotype (GD3 epitope) IgG antibody (InClone System); IMC-C225 which is a chimeric anti-EGFR IgG antibody (InClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DIAGNOSTIC REAGENT antibody (Techniclone); ABX-IL8 is a human anti-IL8 antibody (Abgenix); anti-CD11a is a humanized IgG1 antibody (Genentech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G.11 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); SCH55700 is a humanized anti-IL-5 IgG4 antibody (Celltech/Schering); SB-240563 and SB-240683 are humanized anti-IL-5 and IL-4 antibodies, respectively, (SmithKline Beecham); rhuMab-E25 is a humanized anti-IgE IgG1 antibody (Genentech/Norvartis/Tanox Biosystems); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β₂-integrin IgG antibody (LeukoSite); Anti-LFA-1 is a murine anti CD18 F(ab′)₂ (Pasteur-Merieux/Immunotech); CAT-152 is a human anti-TGF-β₂ antibody (Cambridge Ab Tech); and Corsevin M is a chimeric anti-Factor VII antibody (Centocor). The above-listed immunoreactive reagents, as well as any other immunoreactive reagents, may be administered according to any regimen known to those of skill in the art, including the regimens recommended by the suppliers of the immunoreactive reagents.

In another embodiment, complexes of the invention are administered in combination with one or more anti-angiogenic agents, including, but not limited to, angiostatin, thalidomide, kringle 5, endostatin, Serpin (Serine Protease Inhibitor) anti-thrombin, 29 kDa N-terminal and a 40 kDa C-terminal proteolytic fragments of fibronectin, 16 kDa proteolytic fragment of prolactin, 7.8 kDa proteolytic fragment of platelet factor-4, a 13-amino acid peptide corresponding to a fragment of platelet factor-4 (Maione et al., 1990, Cancer Res. 51:2077-2083), a 14-amino acid peptide corresponding to a fragment of collagen I (Tolma et al., 1993, J. Cell Biol. 122:497-511), a 19 amino acid peptide corresponding to a fragment of Thrombospondin I (Tolsma et al., 1993, J. Cell Biol. 122:497-511), a 20-amino acid peptide corresponding to a fragment of SPARC (Sage et al., 1995, J. Cell. Biochem. 57:1329-1334), or any fragments, family members, or variants thereof, including pharmaceutically acceptable salts thereof.

Other peptides that inhibit angiogenesis and correspond to fragments of laminin, fibronectin, procollagen, and EGF have also been described (see, e.g., Cao, 1998, Prog Mol Subcell Biol. 20:161-176). Monoclonal antibodies and cyclic pentapeptides, which block certain integrins that bind RGD proteins (i.e., possess the peptide motif Arg-Gly-Asp), have been demonstrated to have anti-vascularization activities (Brooks et al., 1994, Science 264:569-571; Hammes et al., 1996, Nature Medicine 2:529-533). Moreover, inhibition of the urokinase plasminogen activator receptor by receptor antagonists inhibits angiogenesis, tumor growth and metastasis (Min et al., 1996, Cancer Res. 56: 2428-33; Crowley et al., 1993, Proc Natl Acad. Sci. 90:5021-25). Use of such anti-angiogenic agents in combination with the complexes is also contemplated by the present invention.

In yet another embodiment, complexes of the invention are used in association with a hormonal treatment. Hormonal therapeutic treatments comprise hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), and antiandrogens (e.g., cyproterone acetate).

In yet another embodiment, complexes of the invention are used in association with a gene therapy program in the treatment of cancer. In one embodiment, gene therapy with recombinant cells secreting interleukin-2 is administered in combination with complexes of the invention to prevent or treat cancer, particularly breast cancer (See, e.g., Deshmukh et al., 2001, J Neurosurg. 94:287-92). In other embodiments, gene therapy is conducted with the use of polynucleotide compounds, including, but not limited to, antisense polynucleotides, ribozymes, RNA interference molecules, triple helix polynucleotides and the like, where the nucleotide sequence of such compounds are related to the nucleotide sequences of DNA and/or RNA of genes that are linked to the initiation, progression, and/or pathology of a tumor or cancer. For example, many are oncogenes, growth factor genes, growth factor receptor genes, cell cycle genes, DNA repair genes, and are well known in the art.

In another embodiment, complexes of the invention are administered in conjunction with a regimen of radiation therapy. For radiation treatment, the radiation can be gamma rays or X-rays. The methods encompass treatment of cancer comprising radiation therapy, such as external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J.B. Lippencott Company, Philadelphia. In preferred embodiments, the radiation treatment is administered as external beam radiation or teletherapy wherein the radiation is directed from a remote source. In various preferred embodiments, the radiation treatment is administered as internal therapy or brachytherapy wherein a radiaoactive source is placed inside the body close to cancer cells or a tumor mass. Also encompassed is the combined use of complexes of the invention with photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, complexes of the invention are administered in combination with at least one chemotherapeutic agent for a short treatment cycle to a cancer patient to treat cancer. The duration of treatment with the chemotherapeutic agent may vary according to the particular cancer therapeutic agent used. The invention also contemplates discontinuous administration or daily doses divided into several partial administrations. An appropriate treatment time for a particular cancer therapeutic agent will be appreciated by the skilled artisan, and the invention contemplates the continued assessment of optimal treatment schedules for each cancer therapeutic agent. The present invention contemplates at least one cycle, preferably more than one cycle during which a single therapeutic or sequence of therapeutics is administered. An appropriate period of time for one cycle will be appreciated by the skilled artisan, as will the total number of cycles, and the interval between cycles.

In another embodiment, complexes of the invention are used in combination with compounds that ameliorate the symptoms of the cancer (such as but not limited to pain) and the side effects produced by the complexes of the invention (such as but not limited to flu-like symptoms, fever, etc). Accordingly, many compounds known to reduce pain, flu-like symptoms, and fever can be used in combination or in admixture with complexes of the invention. Such compounds include analgesics (e.g, acetaminophen), decongestants (e.g., pseudoephedrine), antihistamines (e.g., chlorpheniramine maleate), and cough suppressants (e.g., dextromethorphan).

5.18.2 Treatment of Infectious Diseases

Infectious diseases that can be treated or prevented by the methods of the present invention are caused by infectious agents including, but not limited to, viruses, bacteria, fungi protozoa, helminths, and parasites. The invention is not limited to treating or preventing infectious diseases caused by intracellular pathogens. Many medically relevant microorganisms have been described extensively in the literature, e.g., see C. G. A Thomas, Medical Microbiology, Bailliere Tindall, Great Britain 1983, the entire contents of which is hereby incorporated by reference.

Combination therapy encompasses in addition to the administration of complexes of the invention, the uses of one or more modalities that aid in the prevention or treatment of infectious diseases, which modalities include, but is not limited to antibiotics, antivirals, antiprotozoal compounds, antifingal compounds, and antihelminthics. Other treatment modalities that can be used to treat or prevent infectious diseases include immunotherapeutics, polynucleotides, antibodies, cytokines, and hormones as described above.

Infectious viruses of both human and non-human vertebrates, include retroviruses, RNA viruses and DNA viruses. Examples of virus that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP, Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1: internally transmitted; class 2: parenterally transmitted (i.e. Hepatitis C); Norwalk and related viruses, and astroviruses).

Retroviruses that are contemplated include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyainwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Many examples of antiviral compounds that can be used in combination with the complexes of the invention are known in the art and include, but are not limited to: rifampicin, nucleoside reverse transcriptase inhibitors (e.g., AZT, ddI, ddC, 3TC, d4T), non-nucleoside reverse transcriptase inhibitors (e.g., Efavirenz, Nevirapine), protease inhibitors (e.g., lopinavir, amprenavir, indinavir, ritonavir, and saquinavir), idoxuridine, cidofovir, acyclovir, ganciclovir, zanamivir, amantadine, and Palivizumab. Other examples of anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime.

Bacterial infections or diseases that can be treated or prevented by the methods of the present invention can be caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include, but are not limited to, infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species. Specific examples of infectious bacteria include but are not limited to: Helicobacter pyloris, Borelia burgdorferi, Legionella, pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, Corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Antibacterial agents or antibiotics that can be used in combination with the complexes of the invention include, but are not limited to: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

Additional examples of antibacterial agents include, but are not limited to, Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carunonam Sodium; Cefaclor; Cefadroxil; Cefamandole; Cefarnandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmnenoxime Hydrochloride; Cefinetazole; Cefinetazole Sodium; Cefonicid Monosodium; Cefonicid Sodium; Cefoperazone Sodium; Ceforamide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafingin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Fungal diseases that can be treated or prevented by the methods of the present invention include, but not limited to, aspergilliosis, crytococcosis, sporotrichosis, coccidioidomycosis, paracoccidioidomycosis, histoplasmosis, blastomycosis, zygomycosis, and candidiasis.

Antifungal compounds that can be used in combination with the complexes of the invention include, but are not limited to: polyenes (e.g., amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include, but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefingin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochloride.

Parasitic diseases that can be treated or prevented by the methods of the present invention include, but are not limited to, amebiasis, malaria, leishmania, coccidia, giardiasis, cryptosporidiosis, toxoplasmosis, and trypanosomiasis. Also encompassed are infections by various worms, including, but not limited to, ascariasis, ancylostomiasis, trichuriasis, strongyloidiasis, toxoccariasis, trichinosis, onchocerciasis. filaria, and dirofilariasis. Also encompassed are infections by various flukes, such as but not limited to schistosomiasis, paragonimiasis, and clonorchiasis. Parasites that cause these diseases can be classified based on whether they are intracellular or extracellular. An “intracellular parasite” as used herein is a parasite whose entire life cycle is intracellular. Examples of human intracellular parasites include Leishimania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites.” These parasites may exist most of their lives or only a small portion of their lives in an extracellular environment, but they all have at least one obligate intracellular stage in their life cycles. This latter category of parasites includes Trypanosoma rhodesiense and Trypanosoma gambiense; Isospora spp., Cryptosporidium spp, Eirneria spp., Neospora spp., Sarcocystis spp., and Schistosoma spp.

Many examples of antiprotozoal compounds that can be used in combination with the complexes of the invention to treat parasitic diseases are known in the art and include, but are not limited to: quinines, chloroquine, mefloquine, proguanil, pyrimethamine, metronidazole, diloxanide furoate, tinidazole, amphotericin, sodium stibogluconate, trimoxazole, and pentamidine isetionate. Many examples of antiparasite drugs that can be used in combination with the complexes of the invention to treat parasitic diseases are known in the art and include, but are not limited to: mebendazole, levamisole, niclosamide, praziquantel, albendazole, ivermectin, diethylcarbamazine, and thiabendazole. Further examples of anti-parasitic compounds include, but are not limited to Acedapsone; Amodiaquine Hydrochloride; Amquinate; Arteflene; Chloroquine; Chloroquine Hydrochloride; Chloroquine Phosphate; Cycloguanil Pamoate; Enpiroline Phosphate; Halofantrine Hydrochloride; Hydroxychloroquine Sulfate; Mefloquine Hydrochloride; Menoctone; Mirincamycin Hydrochloride; Primaquine Phosphate; Pyrimethamine; Quinine Sulfate; and Tebuquine.

In one embodiment, the complexes of the invention can be used in combination with a non-hsp vaccine composition. Examples of such vaccines for humans are described in The Jordan Report 2000, Accelerated Development of Vaccines, National Institute of Health, which is incorporated herein by reference in its entirety. Many vaccines for the treatment of non-human vertebrates are disclosed in Bennett, K. Compendium of Veterinary Products, 3rd ed. North American Compendiums, Inc., 1995, which is incorporated herein by reference in its entirety.

5.18.3 Autologous Embodiment

The specific immunogenicity of hsps are derived not from the hsps per se, but from the antigenic proteins and/or peptides bound to them. In a preferred embodiment of the invention, the complexes in the compositions of the inventions for use as cancer vaccines are autologous complexes, thereby circumventing two of the most intractable hurdles to cancer immunotherapy. First is the possibility that human cancers, like cancers of experimental animals, are antigenically distinct. To circumvent this hurdle, in a preferred embodiment of the present invention, the hsps are complexed to antigenic proteins and peptides, and the complexes are used to treat the cancers in the same subject from which the proteins or peptides are derived. Second, most current approaches to cancer immunotherapy focus on determining the CTL-recognized epitopes of cancer cell lines. This approach requires the availability of cell lines and CTLs against cancers. These reagents are unavailable for an overwhelming proportion of human cancers. In an embodiment of the present invention directed to the use of autologous antigenic proteins and/or peptides, cancer immunotherapy does not depend on the availability of cell lines or CTLs nor does it require definition of the antigenic epitopes of cancer cells. These advantages make complexes of hsps bound to autologous antigenic proteins and/or peptides attractive immunogens against cancer.

In other embodiments, the antigenic peptides in the therapeutic or prophylactic complexes can be prepared from cancerous tissue of the same type of cancer from a subject allogeneic to the subject to whom the complexes are administered.

5.19 Kits, Dosage Regimens, Administration and Formulations

The complexes of antigenic proteins/peptides bound to heat shock proteins prepared by the methods of the invention can be administered to a patient in therapeutically effective doses to treat or ameliorate a cell proliferative disorder or infectious disease. A therapeutically effective dose refers to that amount of the complexes sufficient to result in amelioration of symptoms of such a disorder. The effective dose of the complexes may be different when another treatment modality is being used in combination. The appropriate and recommended dosages, formulation and routes of administration for treatment modalities such as chemotherapeutic agents, radiation therapy and biological/immunotherapeutic agents such as cytokines are known in the art and described in such literature as the Physician's Desk Reference (56^{th} ed., 2002).

5.19.1 Effective Dose

The compositions of the present invention, comprising an immunogenic, effective amount of complexes of a population of antigenic peptides with heat shock proteins can be administered to a subject in need of treatment against cancer or an infectious disease as a method of inducing an immune response against that cancer or infectious disease. Toxicity and therapeutic efficacy of such complexes can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Complexes that exhibit large therapeutic indices are preferred. While complexes that exhibit toxic side effects can be used, care should be taken to design a delivery system that targets such complexes to the site of affected tissue in order to minimize potential damage to uninfected cells and, thereby, reduce side effects.

In one embodiment, the data obtained from the cell culture assays and animal studies can be used in formulating a range of dosage for use in humans. The dosage of complexes lies preferably within a range of circulating concentrations that include the ED₅₀ with little or no toxicity. The dosage may vary within this range depending upon the dosage form employed and the route of administration utilized. For any complexes used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In another embodiment, hsp70- and/or gp96-antigenic molecule complexes, or a combination thereof, can be administered that is in the range of about 0.1 μg to about 600 μg, and preferably about 1 μg to about 60 μg for a human patient. The amount of hsp70- and/or gp96 complexes administered can be 0.1, 0.2, 0.3, 0.5, 0.7, 0.8, 1, 2, 5, 10, 15, 20, 25, 30, 35, 40, 45, 50, 60, 70, 80, 90, 100, 125, 150, 175, 200, 250, 300, 350, 400, 450, 500, 550 or 600 μg. Preferably, the amount is less than 100 μg. Most preferably, the amount of hsp70- and/or gp96 complexes administered is 5 μg, 251 μg, or 50 μg. The dosage for hsp-90 peptide complexes in a human patient provided by the present invention is in the range of about 5 to 5,000 μg. Preferably, the the amount of hsp90 complexes administered is 5, 10, 15, 20, 25, 30, 40, 50, 60, 70, 80, 90, 100, 150, 200, 250, 300, 400, 500, 750, 1000, 1500, 2000, 2500, 3000, 4000 or 5000 μg, the most preferred dosage being 100 μg. These doses are preferably administered intradermally or subcutaneously. These doses can be given once or repeatedly, such as daily, every other day, weekly, biweekly, or monthly. Preferably, the complexes are given once weekly for a period of about 4-6 weeks, and the mode or site of administration is preferably varied with each administration. Thus, by way of example and not limitation, the first injection can be given subcutaneously on the left arm, the second on the right arm, the third on the left belly, the fourth on the right belly, the fifth on the left thigh, the sixth on the right thigh, etc. The same site may be repeated after a gap of one or more injections. Also, split injections may be given. Thus, for example, half the dose may be given in one site and the other half in another site on the same day. Alternatively, the mode of administration is sequentially varied, e.g., weekly injections are given in sequence intradermally, intramuscularly, subcutaneously, intravenously or intraperitoneally. Preferably, the once weekly dose is given for a period of 4 weeks. After 4-6 weeks, further injections are preferably given at two-week intervals over a period of time of one or more months, or until supply of complexes is exhausted. Later injections may be given monthly. The pace of later injections can be modified, depending upon the patient's clinical progress and responsiveness to the immunotherapy. In a preferred example, intradermal administrations are given, with each site of administration varied sequentially.

Accordingly, the invention provides methods of preventing and treating cancer or an infectious disease in a subject comprising administering a composition which stimulates the immunocompetence of the host individual and elicits specific immunity against the preneoplastic and/or neoplastic cells or infected cells.

In a specific embodiment, during combination therapy, the hsp-peptide complexes are administered in a sub-optimal amount, e.g., an amount that does not manifest detectable therapeutic benefits when administered in the absence of the therapeutic modality, as determined by methods known in the art. In such methods, the administration of such a sub-optimal amount of hsp-peptide complexes to a subject receiving a therapeutic modality results in an overall improvement in effectiveness of treatment.

In a preferred embodiment, an hsp-peptide complex is administered in an amount that does not result in tumor regression or cancer remission or an amount wherein the cancer cells have not been significantly reduced or have increased when said hsp-peptide complex is administered in the absence of the therapeutic modality. In a preferred embodiment, the sub-optimal amount of hsp-peptide complexes is administered to a subject receiving a treatment modality whereby the overall effectiveness of treatment is improved. Included among the subjects being treated with hsp-peptide complexes are those receiving chemotherapy or radiation therapy. A sub-optimal amount can be determined by appropriate animal studies. Such a sub-optimal amount in humans can be determined by extrapolation from experiments in animals.

In certain specific embodiments, an hsp-peptide complex is administered to a subject already receiving a chemotherapeutic agent, such as GLEEVEC™ (e.g., 400-800 mg daily in capsule form, 400-600 mg doses administered once daily, or 800 mg dose administered daily in two doses of 400 mg each). GLEEVEC™ is used hereinbelow as a non-limiting example of a chemotherapeutic agent that can be used in combination. For many other chemotherapeutic agents, a similar dosing regime can be used. In such embodiments, the appropriate hsp-peptide complex is initially administered to a subject who has already been receiving GLEEVEC™ in the absence of the hsp-peptide complex for 2 days, 2 days to 1 week, 1 week to 1 month, 1 month to 6 months or 6 months to 1 year prior to administration of the hsp-peptide complex in addition to GLEEVEC™. In a specific embodiment, the hsp-peptide complex is administered to a subject wherein the subject showed resistance to treatment with GLEEVEC™ alone.

In other embodiments, the hsp-peptide complexes are initially administered to a subject concurrently with the initial administration of GLEEVEC™.

In yet other specific embodiments, GLEEVEC™ (e.g., 400-800 mg daily in capsule form) is administered to a subject already receiving treatment comprising administration of hsp-peptide complexes. In such embodiments, GLEEVEC™ is initially administered to a subject who has already been receiving hsp-peptide complexes in the absence of GLEEVEC™ for 2 days, 2 days to 1 week, 1 week to 1 month, 1 month to 6 months, 6 months to 1 year prior to administration of GLEEVEC™ in addition to administration of the hsp-peptide complexes.

In a specific embodiment, a chemotherapeutic agent such as GLEEVEC™ is administered orally. In another specific embodiment, the hsp-peptide complexes are administered intradermally.

In each of the methods contemplated above, the subject, by way of example, receives 50 mg to 100 mg, 100 mg to 200 mg, 200 mg to 300 mg, 300 mg to 400 mg, 400 mg to 500 mg, 500 mg to 600 mg, 600 mg to 700 mg, 700 mg to 800 mg, 800 mg to 900 mg, or 900 mg to 1000 mg of chemotherapeutic agents, such as GLEEVEC™, daily. In certain embodiments, the total daily dose is administered to a subject as two daily doses of 25 mg to 50 mg, 50 mg to 100 mg, 100 mg to 200 mg, 200 mg to 300 mg, 300 mg to 400 mg, or 400 mg to 500 mg.

5.19.2 Therapeutic Regimens

For any of the combination therapies described above for treatment or prevention of cancer and infectious diseases, the complexes of the invention can be administered prior to, concurrently with, or subsequent to the administration of the non-hsp based modality. The non-hsp based modality can be any one of the modalities described above for treatment or prevention of cancer or infectious disease.

In one embodiment, the complexes of the invention is administered to a subject at reasonably the same time as the other modality. This method provides that the two administrations are performed within a time frame of less than one minute to about five minutes, or up to about sixty minutes from each other, for example, at the same doctor's visit.

In another embodiment, the complexes of the invention and a modality are administered at exactly the same time. In yet another embodiment the complexes of the invention and the modality are administered in a sequence and within a time interval such that the complexes of the invention and the modality can act together to provide an increased benefit than if they were administered alone. In another embodiment, the complexes of the invention and a modality are administered sufficiently close in time so as to provide the desired therapeutic or prophylactic outcome. Each can be administered simultaneously or separately, in any appropriate form and by any suitable route. In one embodiment, the complexes of the invention and the modality are administered by different routes of administration. In an alternate embodiment, each is administered by the same route of administration. The complexes of the invention can be administered at the same or different sites, e.g. arm and leg. When administered simultaneously, the complexes of the invention and the modality can, but need not, be administered in admixture or at the same site of administration by the same route of administration.

In a preferred embodiment, the complexes of the invention are administered according to the regimen described in Section 5.19.1. In various embodiments, the complexes of the invention and the modality are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In other embodiments, the complexes of the invention and vaccine composition are administered 2 to 4 days apart, 4 to 6 days apart, 1 week a part, 1 to 2 weeks apart, 2 to 4 weeks apart, one moth apart, 1 to 2 months apart, or 2 or more months apart. In preferred embodiments, the complexes of the invention and the modality are administered in a time frame when both are still active. One skilled in the art would be able to determine such a time frame by determining the half life of each administered component.

In one embodiment, the complexes of the invention and the modality are administered within the same patient visit. In a specific preferred embodiment, the complexes of the invention is administered prior to the administration of the modality. In an alternate specific embodiment, the complexes of the invention is administered subsequent to the administration of the modality.

In certain embodiments, the complexes of the invention and the modality are cyclically administered to a subject. Cycling therapy involves the administration of the complexes of the invention for a period of time, followed by the administration of a modality for a period of time and repeating this sequential administration. Cycling therapy can reduce the development of resistance to one or more of the therapies, avoid or reduce the side effects of one of the therapies, and/or improve the efficacy of the treatment. In such embodiments, the invention contemplates the alternating administration of a complexes of the invention followed by the administration of a modality 4 to 6 days later, preferably 2 to 4 days, later, more preferably 1 to 2 days later, wherein such a cycle can be repeated as many times as desired. In certain embodiments, the complexes of the invention and the modality are alternately administered in a cycle of less than 3 weeks, once every two weeks, once every 10 days or once every week. In a specific embodiment, complexes of the invention are administered to a subject within a time frame of one hour to twenty four hours after the administration of a modality. The time frame can be extended further to a few days or more if a slow- or continuous-release type of modality delivery system is used.

5.19.3 Formulations and Use

Pharmaceutical compositions for use in accordance with the present invention can be formulated in a conventional manner using one or more physiologically acceptable carriers or excipients.

Thus, the complexes and their physiologically acceptable salts and solvates can be formulated for administration by inhalation or insufflation (either through the mouth or the nose) oral, buccal, parenteral, rectal, or transdermal administration. Non-invasive methods of administration are also contemplated.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration may take the form of, for example, solutions, syrups or suspensions, or they may be presented as a dry product for constitution with water or other suitable vehicles before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active complexes.

For buccal administration the compositions can take the form of tablets or lozenges formulated in conventional manner.

For administration by inhalation, the complexes for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the complexes and a suitable powder base such as lactose or starch.

The complexes can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and may contain formulatory agents such as suspending, stabilizing and/or dispersing agents. Alternatively, the active ingredient can be in powder form for constitution with a suitable vehicle, e.g., sterile pyrogen-free water, before use.

The complexes can also be formulated in rectal compositions such as suppositories or retention enemas, e.g., containing conventional suppository bases such as cocoa butter or other glycerides.

In addition to the formulations described previously, the complexes can also be formulated as a depot preparation. Such long acting formulations can be administered by implantation (for example subcutaneously or intramuscularly) or by intramuscular injection. Thus, for example, the complexes can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Also encompassed is the use of adjuvants in combination with or in admixture with the complexes of the invention. Adjuvants contemplated include, but are not limited to, mineral salt adjuvants or mineral salt gel adjuvants, particulate adjuvants, microparticulate adjuvants, mucosal adjuvants, and immunostimulatory adjuvants, such as those described in Section 5.18. Adjuvants can be administered to a subject as a mixture with complexes of the invention, or used in combination with the complexes as described in Section 5.19.2.

Also contemplated is the use of adenosine diphosphate (ADP) in combination with or in admixture with the complexes of the invention, preferably gp96 complexes.

5.19.4 Kits

The invention also provides kits for carrying out the methods and/or therapeutic regimens of the invention.

In one embodiment, such kits comprise in one or more containers protein preparations comprising antigenic proteins and peptides for combining with hsp that are provided in a second container. In another embodiment, such kits comprise in one or more containers digested peptides comprising antigenic peptides for combining with hsps that are provided in a second container. Alternatively, proteins and/or peptides can be supplied in one or more containers for complexing to hsps isolated from a specific patient for autologous administration. Optionally, a purified HSP for complexing to proteins and peptides is further provided in a second container.

In another embodiment, such kits can comprise in one or more containers therapeutically or prophylactically effective amounts of the complexed proteins/peptides to hsps, preferably purified, in pharmaceutically acceptable form. The kits optionally further comprise in a second container sensitized APCs, preferably purified.

In another embodiment, the kits can comprise a hsp-peptide complex in one container, an oligomerizing agent for oligomerizing said complex in a second container and instructions for preparation of the oligomerized complex. In another embodiment, the kits can comprise a container comprising an antigenic peptide, another container comprising a hsp, a third container comprising an oligomerizing agent for oligomerizing the hsp of the kit and instructions for preparation of an oligomerized hsp-peptide complex.

The hsp-peptide complexes in a container of a kit of the invention can be in the form of a pharmaceutically acceptable solution, e.g., in combination with sterile saline, dextrose solution, or buffered solution, or other pharmaceutically acceptable sterile fluid. Alternatively, the hsp-peptide complexes can be lyophilized or desiccated; in this instance, the kit optionally further comprises in a container a pharmaceutically acceptable solution (e.g., saline, dextrose solution, etc.), preferably sterile, to reconstitute the hsp or the hsp-containing complexes to form a solution for injection purposes.

In another embodiment, a kit of the invention further comprises a needle or syringe, preferably packaged in sterile form, for injecting the hsp-peptide complex, and/or a packaged alcohol pad. Instructions are optionally included for administration of hsp-peptide complexes by a clinician or by the patient.

Kits are also provided for carrying out the combination therapies of the present invention. In one embodiment, a kit comprises a first container containing one or more purified hsp-peptide complexes and a second container containing a non-HSP based therapeutic modality for treatment of cancer. Preferably, the cancer is CML, the hsp-peptide complexes comprises hsp70-peptide complexes, and the therapeutic modality is GLEEVEC™. In a specific embodiment, the second container contains imatinib mesylate. In another specific embodiment, the imatinib mesylate is purified.

In a specific embodiment, a kit comprises a first container containing one or more purified hsp-peptide complexes in an amount ineffective to treat a disease or disorder when administered alone; and a second container containing a non-hsp based treatment modality in an amount that, when administered before, concurrently with, or after the administration of the hsp-peptide complexes in the first container, is effective to improve overall treatment effectiveness over the effectiveness of the administration of each component alone. In another specific embodiment, a kit comprises a first container containing one or more purified hsp-peptide complexes in an amount ineffective to treat a disease or disorder when administered alone; and a second container containing one or more non-hsp based treatment modalities in an amount that, when administered before, concurrently with, or after the administration of the hsp-peptide complexes in the first container, is effective to improve overall treatment effectiveness over the effectiveness of the administration of the hsp-peptide complexes administered alone or the treatment modalities administered alone. In yet another specific embodiment, a first container containing one or more purified hsp-peptide complexes in an amount ineffective to treat a disease or disorder when administered alone; and a second container and third container, each containing a non-hsp based treatment modality in an amount that, when administered before, concurrently with, or after the administration of the hsp-peptide complexes in the first container, is effective to improve overall treatment effectiveness over the effectiveness of the administration of hsp-peptide complexes administered alone or treatment modalities administered alone. In a preferred specific embodiment, the invention provides a kit comprising in a first container, one or more purified hsp-peptide complexes comprising a population of noncovalent hsp-peptide complexes of the invention; in a second container, a composition comprising an anti-cancer agent; and in a third container, a composition comprising a cytokine or an adjuvant.

The kit can for example comprise metal or plastic foil, such as a blister pack. The kit can be accompanied by one or more reusable or disposable device(s) for administration (e.g, syringes, needles, dispensing pens) and/or instructions for administration.

5.20 Monitoring of Effects During Immunotherapy

The effect of immunotherapy with hsp-antigenic molecule complexes on development and progression of neoplastic diseases can be monitored by any methods known to one skilled in the art, including but not limited to measuring: a) delayed hypersensitivity as an assessment of cellular immunity; b) activity of cytolytic T-lymphocytes in vitro; c) levels of tumor specific antigens, e.g., carcinoembryonic (CEA) antigens; d) changes in the morphology of tumors using techniques such as a computed tomographic (CT) scan; e) changes in levels of putative biomarkers of risk for a particular cancer in individuals at high risk, and f) changes in the morphology of tumors using a sonogram.

5.20.1 Delayed Hypersensitivity Skin Test

Delayed hypersensitivity skin tests are of great value in the overall immunocompetence and cellular immunity to an antigen. Inability to react to a battery of common skin antigens is termed anergy (Sato, T., et al, 1995, Clin. Immunol. Pathol., 74:35-43).

Proper technique of skin testing requires that the antigens be stored sterile at 4 C, protected from light and reconstituted shorted before use. A 25- or 27-gauge needle ensures intradermal, rather than subcutaneous, administration of antigen. Twenty-four and 48 hours after intradermal administration of the antigen, the largest dimensions of both erythema and induration are measured with a ruler. Hypoactivity to any given antigen or group of antigens is confirmed by testing with higher concentrations of antigen or, in ambiguous circumstances, by a repeat test with an intermediate test.

5.20.2 In Vitro Activation of Cytotoxic T-Cells

8×10⁶ peripheral blood derived T lymphocytes isolated by the Ficoll-Hypaque centrifugation gradient technique, are restimulated with 4×104 mitomycin C treated tumor cells in 3 ml RPMI medium containing 10% fetal calf serum. In some experiments, 33% secondary mixed lymphocyte culture supernatant or IL-2, is included in the culture medium as a source of T-cell growth factors.

In order to measure the primary response of cytolytic T-lymphocytes after immunization, T-cells are cultured without the stimulator tumor cells. In other experiments, T-cells are restimulated with antigenically distinct cells. After six days, the cultures are tested for cytotoxicity in a 4 hour 51Cr-release assay. The spontaneous 51Cr-release of the targets should reach a level less than 20%. For the anti-MHC class I blocking activity, a tenfold concentrated supernatant of W6/32 hybridoma is added to the test at a final concentration of 12.5% (Heike M., et al., J. immunotherapy, 15:165-174).

5.20.3 Levels of Tumor Specific Antigens

Although it may not be possible to detect unique tumor antigens on all tumors, many tumors display antigens that distinguish them from normal cells. The monoclonal antibody reagents have permitted the isolation and biochemical characterization of the antigens and have been invaluable diagnostically for distinction of transformed from nontransformed cells and for definition of the cell lineage of transformed cells. The best-characterized human tumor-associated antigens are the oncofetal antigens. These antigens are expressed during embryogenesis, but are absent or very difficult to detect in normal adult tissue. The prototype antigen is carcinoembryonic antigen (CEA), a glycoprotein found on fetal gut and human colon cancer cells, but not on normal adult colon cells. Since CEA is shed from colon carcinoma cells and found in the serum, it was originally thought that the presence of this antigen in the serum could be used to screen patients for colon cancer. However, patients with other tumors, such as pancreatic and breast cancer, also have elevated serum levels of CEA. Therefore, monitoring the fall and rise of CEA levels in cancer patients undergoing therapy has proven useful for predicting tumor progression and responses to treatment.

Several other oncofetal antigens have been useful for diagnosing and monitoring human tumors, e.g., alpha-fetoprotein, an alpha-globulin normally secreted by fetal liver and yolk sac cells, is found in the serum of patients with liver and germinal cell tumors and can be used as a marker of disease status.

5.20.4 Computed Tomographic (CT) Scan

CT remains the choice of techniques for the accurate staging of cancers. CT has proved more sensitive and specific than any other imaging techniques for the detection of metastases.

5.20.5 Measurement of Putative Biomarkers

The levels of a putative biomarker for risk of a specific cancer are measured to monitor the effect of hsp noncovalently bound to peptide complexes. For example, in individuals at enhanced risk for prostate cancer, serum prostate-specific antigen (PSA) is measured by the procedure described by Brawer, M. K., et. al., 1992, J. Urol., 147:841-845, and Catalona, W. J., et al., 1993, JAMA, 270:948-958; or in individuals at risk for colorectal cancer, CEA is measured as described above; and in individuals at enhanced risk for breast cancer, 16-hydroxylation of estradiol is measured by the procedure described by Schneider, J. et al., 1982, Proc. Natl. Acad. Sci. ISA, 79:3047-3051.

5.20.6 Sonogram

A sonogram remains an alternative choice of technique for the accurate staging of cancers.

### 6. Example: Correlation of Presence of Dimer with In Vitro Bioactivity

The following example provides the basis for the invention:

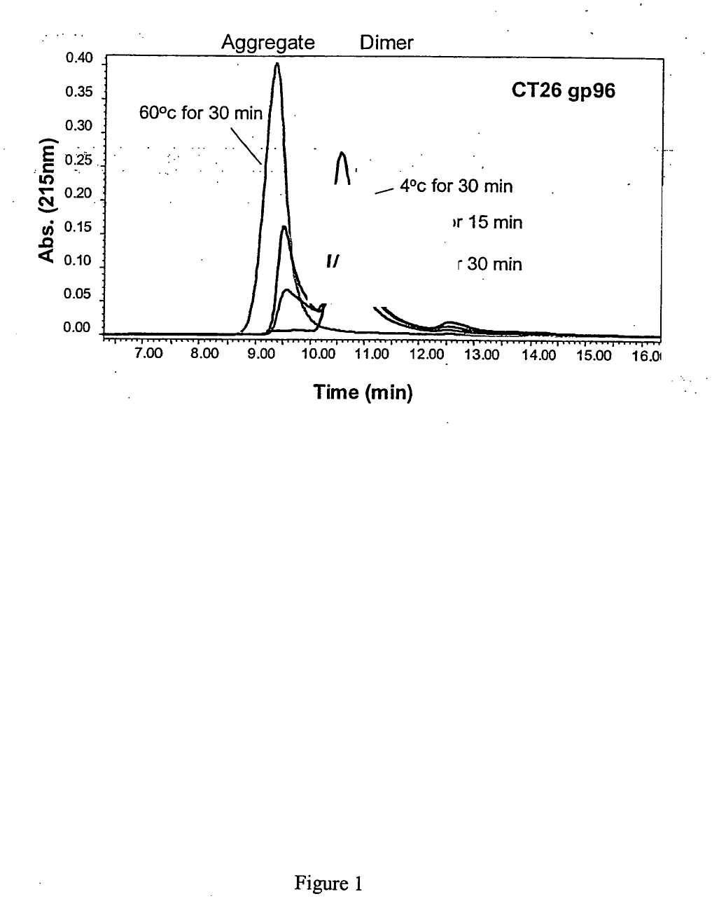

Gp96-peptide complex was isolated from murine CT-26 tumors. Separate aliquots of each preparation were either maintained at 4° C. (untreated) or subjected to mild heat treatment to denature the complex. FIG. 1 shows SEC chromatograms, which show that under the indicated conditions, the protein rapidly converts to higher molecular weight aggregates.

The same samples were then analyzed for antigen re-presentation and T-cell activation as measured by IFN-γ expression. The results of each of three replicates are presented in Table 2, below, which shows that with the thermally induced transition from dimer to aggregate, there was a concomitant loss of antigen re-presentation and T-cell activation.

TABLE 2

| | | | |
|---|---|---|---|
| | | Specific NECA | Tumor specific T- |
| | % | activity | cell activation |
| | Dimer by | (μg NECA bound | (IFN-γ release) |
| Conditions | SEC | gp96/μg gp96) | (pg/ml) |

Experiment 1

| | | | |
|---|---|---|---|
| 4° C. | 75.8 | 3.27 | 102600 |
| 60° C. at 60 min | 0 | 0.08 | 9350 |
| 60° C. at 120 min | 0 | 0.10 | 8100 |

Experiment 2

| | | | |
|---|---|---|---|
| 4° C. | 84.44 | 1.68 | 19600 |
| 49.5° C. at 30 min | 0 | 0.12 | 3150 |
| 60° C. at 30 min | 0 | 0.04 | 50 |

Experiment 3

| | | | |
|---|---|---|---|
| 4° C. | 87 | 1.96 | 8000 |
| 49° C. at 15 min | 74 | 1.97 | 0 |
| 49° C. at 25 min | 54 | 1.80 | 0 |
| 60° C. at 30 min | 0 | 0.07 | 0 |

Dimer content also correlates with the ability of gp96 to bind NECA. Using the gp96-peptide complex isolated from CT-26 tumors, aliquots of each preparation were also analyzed for NECA ligand binding. The results of each of three relicates are presented in Table 2. With the thermally induced transition from dimer to aggregate, there was a concomitant loss of NECA binding activity.

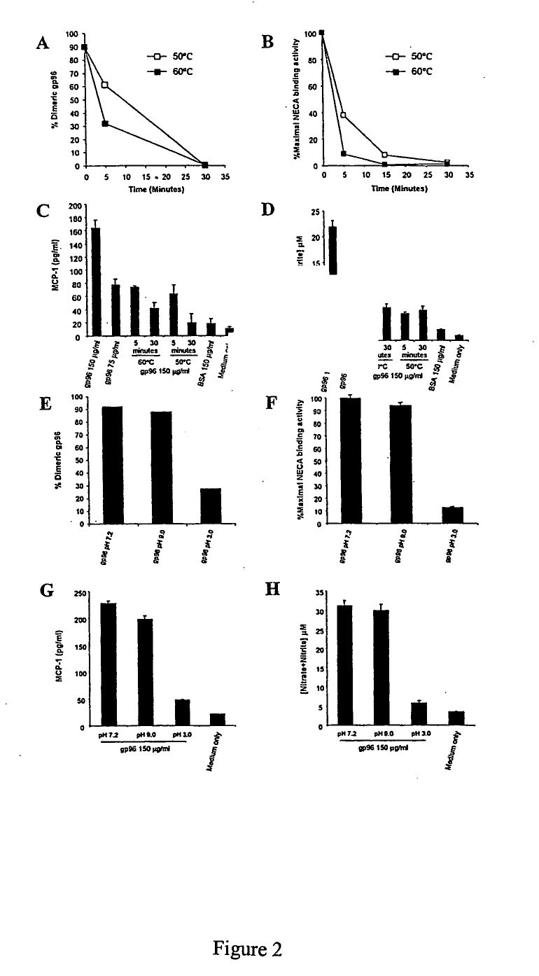

Further experiments indiate that dimer content correlates with the ability of gp96 to stimulate chemokine/nitric oxide release by antigen presenting cells. Gp96 purified from the Meth-A murine fibrosarcoma tumors, subjected to both thermal and pH perturbations. This material was analyzed by SEC and NECA binding for its ability to induce murine antigen presenting cells to secrete MCP-1 and nitric oxide. The results are summarized in FIG. 2. Heat treatment resulted in the conversion of gp96 dimers to higher order aggregates that are believed to be inactive due to denaturation or other conformational changes. NECA ligand binding and both MCP-1 and NO production decreased concomitantly with the loss of dimer. A similar trend was observed upon exposure of gp96 to pH 3, but not pH 7 or 9. These results are consistent with a perceived positive relationship between the structural and biochemical properties of the molecule and biological responses.

### 7. Example: Correlation of Presence of Dimer with ATPase Activity

The results of the following experiments confirm that ATPase activity (measured as a rate of ATP hydrolysis per mg of protein) of purified gp96 complex, directly correlates with the presence of dimer.

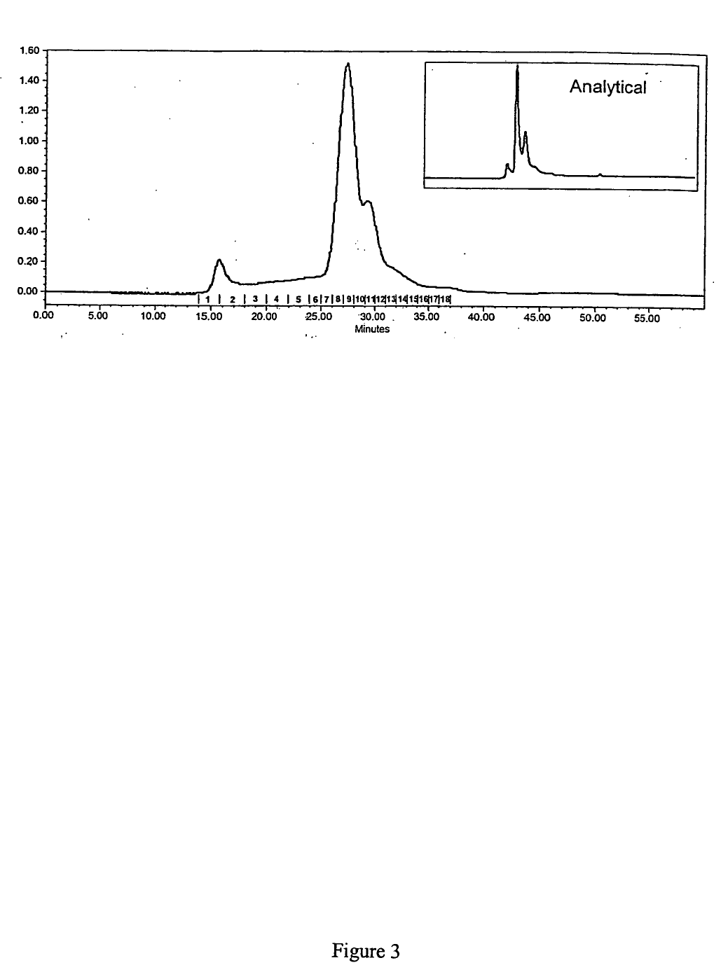

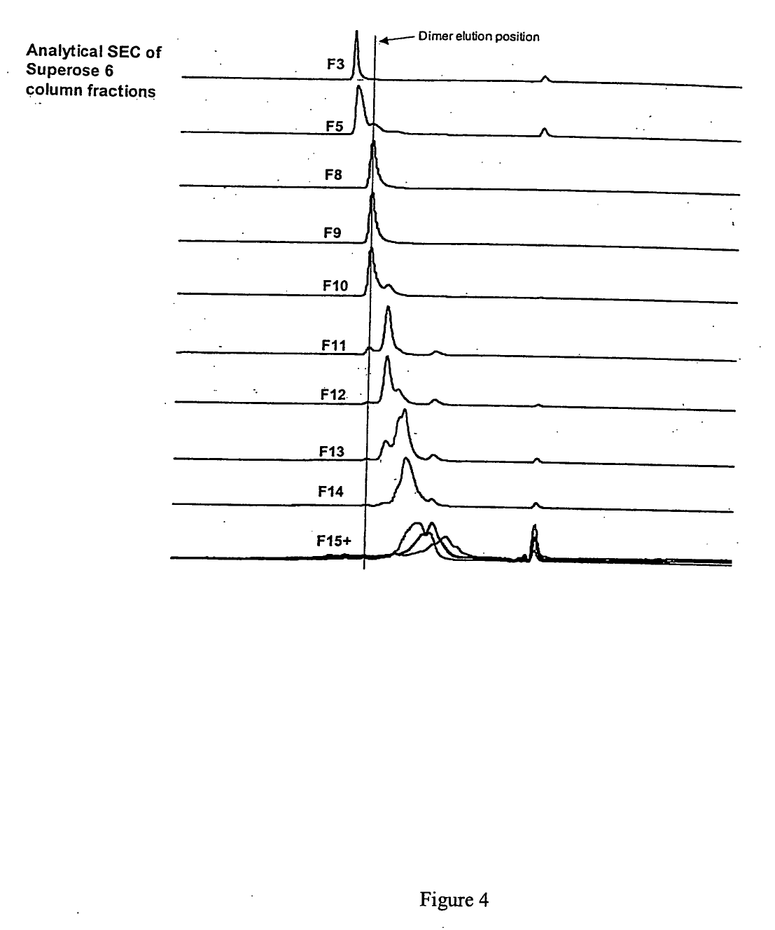

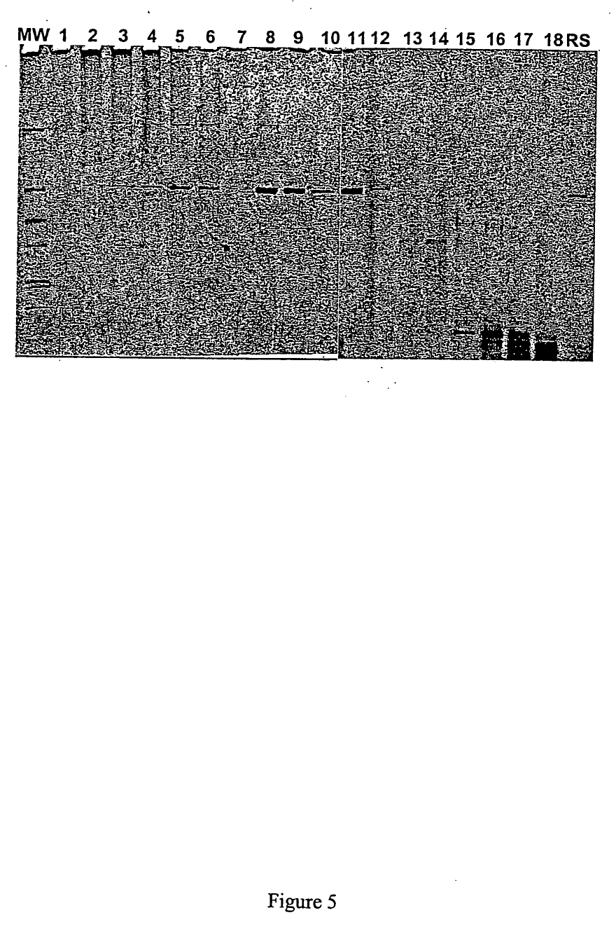

Two sets of experiments were performed with human gp96-peptide complex to determine which of the gp96 isoforms possess ATPase activity. The first set of experiments (FIGS. 3-6) utilized human ovarian tumor derived gp96-peptide complex. FIG. 3 shows the preparative and analytical SEC profile for this preparation. Fractions were collected as indicated and subjected to further analyses. FIG. 4 shows analytical SEC chromatograms (the dotted line indicates the elution position of the dimeric form). FIG. 5 shows a silver stained SDS-PAGE gel of the individual fractions. Although most of the fractions contain predominantly a polypeptide at 96 kDa under denaturing conditions, they clearly resolved into different molecular weight species under non-denaturing conditions on SEC. Fractions 2-5 contain mostly higher molecular weight aggregates, while fractions 8-10 represent the dimer. Fractions 11 and 12 are believed to contain the monomeric species based on the SDS-PAGE analysis. Fractions 13 and beyond contain degradation products and lower molecular weight species.

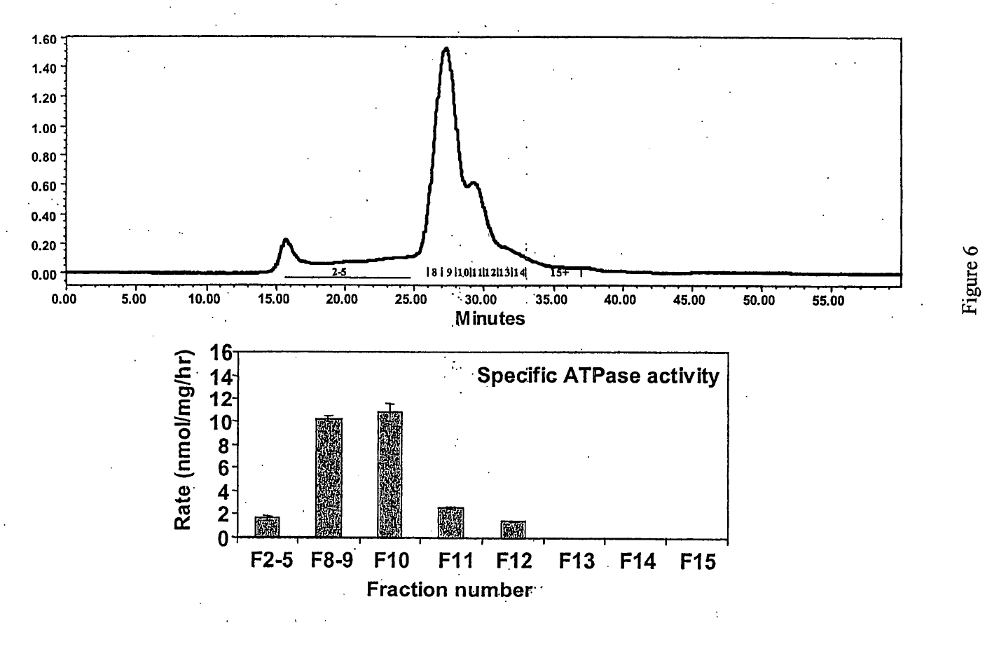

Finally, ATPase activity of each fraction was determined based on the loss of ATP using a luciferin-luciferase luminescence assay. Values are reported as the rate of ATP hydrolysis per mg protein. As shown in FIG. 6, ATPase activity directly correlates with the presence of dimer. The ATPase activity was in the dimer containing fractions with minimal activity in the high molecular weight aggregate and monomer fractions. These results indicate that dimeric gp96 is necessary for the enzymatic hydrolysis of ATP.

These experiments were repeated with a sample of renal tumor derived gp96-peptide complex. Results were entirely consistent with those described above and reinforce the conclusion that dimeric gp96-peptide complex possesses ATPase activity and not monomeric gp96 or the high molecular weight aggregates produced during purification (in the absence of cross-linking agents) which are believed to be inactive due to denaturation or other conformational changes.

### 8. Example: Correlation of ATPase Activity with Antigen Re-Presentation

In an effort to demonstrate the relevance of ATPase activity as a biological measure of potency, a set of experiments were initiated with human gp96 to correlate ATPase activity with antigen re-presentation. These experiments were intended to build on the data linking dimeric gp96 with antigen re-presentation in the mouse model and the data linking dimeric gp96 with ATPase activity in a human derived complex.

For this purpose, a hybrid antigen re-presentation bioassay system was developed in which the mouse antigen, AH1 peptide, for which a mouse CD8 T-cell line is available, was exchanged in vitro onto human tumor derived hsp. The gp96-peptide complex was then tested for its ability to facilitate antigen re-presentation and antigen-specific T-cell stimulation. Presented below are the results of three experiments with two human tumor types which show a correlation between the assay results for ATPase activity, antigen re-presentation and % dimer by SEC. These results add to our existing knowledge base and provide further support that ATPase activity relates in a meaningful way to other critical biological and structural attributes of hsps and hsp-peptide complexes that are important for their biological activity.

Gp96 preparations were made from human endometrial or renal cell carcinoma tumor samples. Briefly, human tumor tissue was homogenized, clarified by centrifugation and subjected to a 50% ammonium sulfate precipitation. The resulting supernatant was further purified using Con A and DEAE chromatography. Purified protein was stored at −80° C. until use.

The antigen presenting cell line, RAW264.7 (ATCC #TIB-71), is an Abelson murine leukemia virus transformed macrophage cell line, which originated from the BALB/c strain (H-2^{d}). The CTL line specific for the murine CT26 tumor derived AH1 epitope was obtained from P. Srivastava (U. of Conn. Medical Center). They were re-stimulated on a weekly basis with the AH1 peptide plus irradiated BALB/c splenocytes. The 9 amino acid AH1 peptide epitope (SPSYVYHQF) and the 19mer peptide (RVTYHSPSYVYHQFERRAK) which contained this epitope were obtained from Sigma Genosys 1442 lake Front Circle, The Woodlands, Tex. 77380-3600.

AH1 19mer peptide was added to purified gp96 preparations at a ratio of 50:1 peptide to protein and the mixture incubated at 37° C. for 30 mm. Unbound peptide was removed by 4 cycles of spin dialysis with 10 volumes of PBS using 30K MWCO Centricon spin filter units (Millipore). Total protein was determined by the Bradford assay.

The in vitro antigen re-presentation assay has been described. Briefly, 10⁴ AH1 peptide-specific CTLs (day 8 post stimulation) were co-cultured with 10⁴ RAW264.7 cells in 96-well flat bottom plates. In vitro loaded gp96 samples at 10 μg/mL were analyzed neat or diluted with unloaded gp96 at the indicated amounts in order to maintain constant protein concentration. Protein samples and controls were added to the wells and incubated at 37° C. for 18 hr. Following centrifugation, the supernatants were harvested for analysis of IFN-γ levels by ELISA (R&D Systems). Reported values represent the average of six replicates. AH1 9mer peptide served as a positive control and unloaded gp96, AH1 19mer peptide and media alone as negative controls. Additional negative controls were provided by applying identical sets of samples to plates that contained either CTLs or APCs only.

ATPase activity was measured as the loss of ATP following 4 hr. of incubation at 37° C. using a luciferin-luciferase luminescence assay. Values are reported as the rate of ATP hydrolysis per mg protein and represent only the activity that is geldanamycin inhibitable to ensure specificity for the hsp90 family of heat shock proteins.

The assay is performed as follows. Gp96 samples (2 μL, 500 μg/mL in PBS) were mixed with 2 μL of a solution comprising a 10 fold molar excess ATP in PBS containing 12.5 mM MgCl₂, and either 20% (v/v) DMSO or DMSO containing 400 M geldanamycin. This mixture was incubated at 37° C. for 4 hr. Samples were then diluted to 100 μL with PBS and the ATP contained in 50 μL of each sample was quantified using the ROCHE CLSII bioluminescence ATP assay kit. Bioluminescence was measured using a Molecular Devices Lmax luminometer. The difference in ATP concentration between uninhibited and Geldanamycin-inhibited measurements reflects the amount of ATP hydrolysis specific to gp96. Rates were calculated from this differential and expressed as nmoles of ATP hydrolyzed per mg protein per hr.

HPLC size exclusion chromatography is currently being optimized and has been run under a few different formats. The column was either a TSK 3000SWXL (Toso Haus) or a Superose 6 (Amersham Pharmacia) equilibrated in 10 mM phosphate buffer, pH 7.1, with 300 mM NaCl. All experiments were run at room temperature on a Waters Alliance or HP 1100 HPLC systems at a flow rate of 0.5 mL/min. The following results for human tumor derived gp96 (reported in Table 3, below) were all analyzed on the Superose 6 column. % dimer values represent the area percent of the dimer peak relative to total peak area.

TABLE 3

| | | Tumor | Antigen | | ATPase | | | |
|---|---|---|---|---|---|---|---|---|
| | | type | Re-presentation | | (Nmol/mg/hr) | | % Dimer | |
| Exp | Date | (prep date) | untreated | heated | untreated | heated | untreated | heated |
| 1 | Oct. 12, 2001 | human | Pos. | Neg. | 8.5 ± 0.3 | 0 | 86 | 0 |
| | | endometrial | | | | | | |
| | | (Oct. 10, 2001) | | | | | | |
| 2 | Oct. 19, 2001 | human | Pos. | Neg. | 9.4 ± 0.5 | 0 | 83 | 0 |
| | | endometrial | | | | | | |
| | | (Oct. 10, 2001) | | | | | | |
| 3 | Oct. 19, 2001 | human renal | Pos. | Neg. | 10.3 ± 0.1 | 0 | 84 | 0 |
| | | (Oct. 17, 2001) | | | | | | |
| Summary of antigen re-presentation results, ATPase and % Dimer by size exclusion chromatography for human tumor derived gp96 preparations. Gp96 samples were loaded with AH1 19mer peptide, | | | | | | | | |
| # washed to remove unbound peptide then divided into two aliquots, one of which was heated at 60° C. for 10 min. Untreated and heat treated samples were evaluated in respective assays. “Positive” | | | | | | | | |
| # means detection of IFN-γ release, and “negative” means no detection of IFN-γ release. | | | | | | | | |

Heat treated samples were incubated at 60° C. for 10 minutes in a thermocycler or circulating water bath prior to analysis.

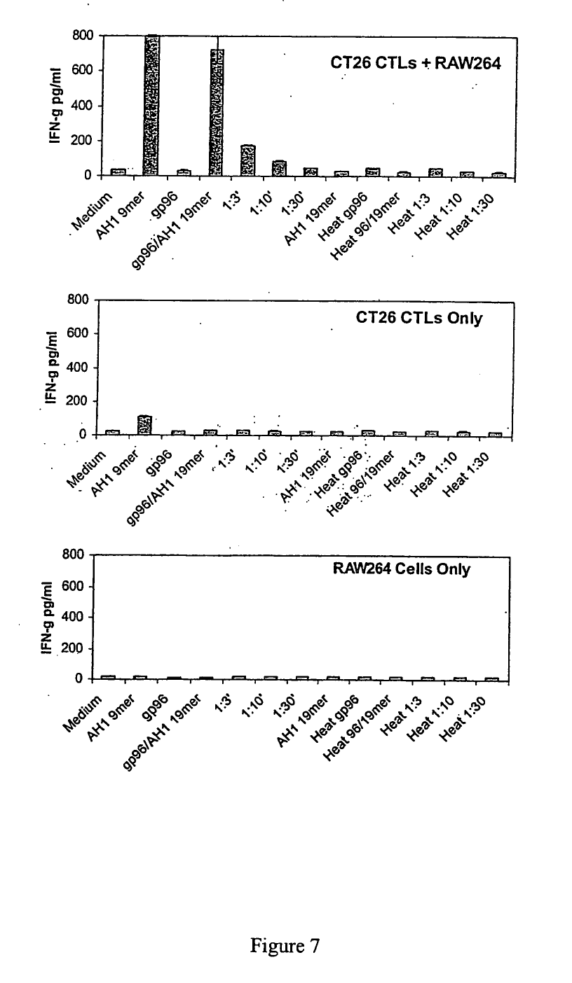

The hybrid re-presentation assay measures the ability of human gp96, loaded with AH1 19mer peptide, to deliver antigen to mouse APCs to facilitate re-presentation leading to simulation of antigen specific T-cells. The results of an experiment using human endometrial derived gp96 are presented in FIG. 7. The top panel shows IFN-γ levels for samples applied to both APCs and CTLs. The second and third panels are controls for CTLs and APCs alone. Each data set contains media alone, unloaded gp96 and AH1 19mer as negative controls and AH1 9mer peptide, which can exchange directly onto surface MHC I molecules, as the positive control. Gp96/AH1 19mer complex was applied at 10 μg/mL or diluted with unloaded gp96 at 1:3, 1:10 or 1:30. The results show a dose dependent IFN-γ response and no stimulation by unloaded gp96. Controls indicate that the activity is specific for AH1 19mer-loaded gp96. When the protein-peptide complex samples were heated at 60° C. for 10 min, conditions known to cause complete aggregation of the protein, the ability to stimulate T-cells was abolished. The experiment was repeated with the same material and identical results were obtained.

The same experimental protocol was also repeated using gp96 protein prepared from a human renal cell carcinoma specimen. Again, identical results were obtained in which there was specific and dose dependent stimulation of T-cells that was abrogated by heat treatment.

Gp96-peptide complex samples from the re-presentation assays were further analyzed for ATPase activity and % dimer by SEC. The results are summarized in Table 3. Samples that were positive in the antigen re-presentation assay possessed ATPase activity and contained predominantly dimer by SEC. Following heat treatment, all ATPase activity was lost and all dimeric protein was converted to high molecular weight aggregates. These results indicate a positive correlation between enzymatic activity measured by ATPase activity, structural attributes measured by SEC, and in vitro biological activity.

### 9. Example: ATPase Activity is Stability Indicating

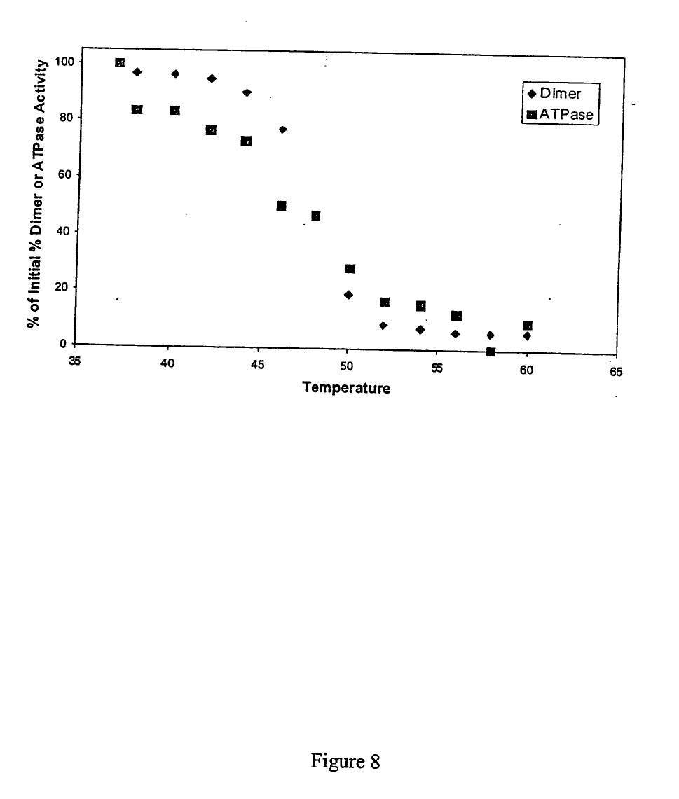

The data presented herein indicate that ATPase activity is a stability-indicating assay. Accelerated stability studies with the dimeric form of gp96 have resulted in consistent, concomitant loss in ATPase activity. These experiments have included mild heat treatments to force aggregation of gp96 as well as proteolytic degradation to generate breakdown products. FIG. 8 shows the results of an experiment in which samples of recombinant gp96 were incubated at the indicated temperatures for 10 minutes, then analyzed for ATPase activity and % dimer by SEC. There was a direct correlation between the loss of ATPase activity and dimeric gp96 as a result of temperature induced denaturation. Similar results were obtained using human gp96-peptide complex, where mild heat treatment both abolished ATPase activity and converted dimeric protein to high molecular weight aggregates.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for detecting the biological activity of heat shock protein-peptide complex comprising using the ATPase activity of the heat shock protein-peptide complex as an indicator of the biological activity of the heat shock protein-peptide complex.

2. A method for detecting the biological activity gp96-peptide complex comprising using the ATPase activity of the gp96-peptide complex as an indicator of the biological activity of the gp96-peptide complex.

3. A method for detecting the biological activity of heat shock protein-peptide complex comprising using the presence of the dimeric form of the heat shock protein-peptide complex as an indicator of the biological activity of the heat shock protein-peptide complex.

4. A method for detecting the biological activity gp96-peptide complex comprising using the presence of the dimeric form of the gp96-peptide complex as an indicator of the biological activity of the gp96-peptide complex.

5. A method for screening a compound that modulates the biological activity of a heat shock protein-peptide complex, comprising:
(A) measuring the ATPase activity of the heat shock protein-peptide complex in the absence of a compound;
(B) contacting the heat shock protein-peptide complex with a compound;
(C) comparing the ATPase activity of the heat shock protein-peptide complex not contacted with the compound with the ATPase activity of the heat shock protein-peptide complex contacted with the compound; and
(D) using any difference in the ATPase activity of the heat shock protein-peptide complex contacted with the compound and the heat shock protein-peptide complex not contacted with the compound as an indicator that the compound modulates the biological activity.

6. The method of claim 5, wherein the ATPase activity is determined by ion exchange chromatography, bioluminescence assay, HPLC, radioisotopic assays or an immunoaffinity assay.

7. The method of claim 5, further comprising determining the ATPase activity in the presence of an inhibitor of nucleotide-hsp binding, wherein the ATPase activity that is inhibited by the presence of the inhibitor is used as an indicator of the biological activity.

8. The method of claim 7, wherein the inhibitor of nucleotide-hsp binding is geldanamycin or NECA.

9. The method of claim 5, further comprising determining the mass of the heat shock protein-peptide complex, such that a specific activity on mass basis of the heat shock protein-peptide complex is provided.

10. A method for screening a compound that modulates the biological activity of a heat shock protein-peptide complex, comprising:
(A) measuring the amount of dimeric form of the heat shock protein-peptide complex in the absence of a compound;
(B) contacting the heat shock protein-peptide complex with a compound;
(C) comparing the amount of dimeric form of the heat shock protein-peptide complex not contacted with the compound with the amount of dimeric form of the heat shock protein-peptide complex contacted with the compound; and
(D) using any difference in the amount of dimeric form of the heat shock protein-peptide complex contacted with the compound and the heat shock protein-peptide complex not contacted with the compound as an indicator that the compound modulates the biological activity.

11. The method of claim 10, wherein the amount of the dimeric form of the heat shock protein-peptide complex is determined by size exclusion chromatography, gel electrophoresis, immunoassay, a filter, light scattering assay, gradient centrifugation or analytical ultracentrifugation.

12. A method for diagnosing a condition in a subject that is due in part to the proper functioning of the immune system, said method comprising using the ATPase activity of heat shock protein-peptide complex or the presence of the dimeric form of heat shock protein-peptide complex obtained from the subject as an indicator of a biological activity of the heat shock protein-peptide complex, wherein the biological activity is associated with one or more immune functions in the subject, whereby a change in ATPase activity or the amounts of dimeric form indicates a change in the condition.

13. A method for determining the prognosis of a cancer or an infectious disease in a subject comprising using the ATPase activity of heat shock protein-peptide complex or the presence of the dimeric form of heat shock protein-peptide complex obtained from the subject as an indicator of a biological activity of the heat shock protein-peptide complex, wherein the biological activity is associated with one or more immune functions that is responsive to the cells of the cancer or the agents that cause the infectious disease, whereby a change in ATPase activity or the amount of dimeric form indicates a change in the prognosis.

14. The method of claim 5, 10, 12 or 13, wherein the hsp in the hsp-peptide complex is gp96.

15. The method of claim 1, 2, 12 or 13, further comprising determining the ATPase activity of the heat shock protein-peptide complex.

16. The method of claim 15, wherein the ATPase activity is determined by a bioluminescence assay, ion exchange chromatography, or an immunoaffinity assay.

17. The method of claim 3, 4, 12 or 13, further comprising determining the amount of the dimeric form of the heat shock protein-peptide complex.

18. The method of claim 17, wherein the amount of the dimeric form of the heat shock protein-peptide complex is determined by size exclusion chromatography, gel electrophoresis, immunoassay, a filter, gradient centrifugation or analytical ultracentrifugation.

19. The method of claim 15, further comprising determining the ATPase activity in the presence of an inhibitor of nucleotide-hsp binding, wherein the ATPase activity that is inhibited by the presence of the inhibitor is used as an indicator of the biological activity.

20. The method of claim 19, wherein the inhibitor of nucleotide-hsp binding is geldanamycin or NECA.

21. The method of claim 15, further comprising determining the mass of the heat shock protein-peptide complex, such that a specific activity on mass basis of the heat shock protein-peptide complex is provided.

22. The method of claim 12 or 13, further comprising isolating and/or purifying the heat shock protein-peptide complex obtained from the subject.

23. A kit comprising a composition comprising heat shock protein-peptide complex wherein the ATPase activity or the amount of the dimeric form of the heat shock protein-peptide complex is used as an indicator of the biological activity of the heat shock protein-peptide complex, and instruction for determining the ATPase activity or the amount of the dimeric form of the heat shock protein-peptide complex.

24. The method of claim 1, 2, 3, 4, 5, 10, 12 or 3 wherein the biological activity is an immunologic activity.

25. The method of claim 24, wherein the immunological activity is antigen re-presentation or T-cell activation.

26. The kit of claim 23, wherein the biological activity is an immunologic activity.

27. The kit of claim 23, wherein the immunological activity is antigen re-presentation or T-cell activation.

28. The method of claim 1, 2, 3, 4, 5, 10, 12 or 13, wherein the biological activity is selected from the group consisting of binding and releasing an antigenic molecule;
inducing MCP-1 production; inducing nitric oxide production; and binding of CD91 or CD36.

29. The kit of claim 23, wherein the biological activity is selected from the group consisting of binding and releasing an antigenic molecule; inducing MCP-1 production; inducing nitric oxide production; and binding of CD91 or CD36.

30. The method of claim 1, 3, 5, 10, 12 or 13, wherein the hsp in the hsp-peptide complex is a member of an hsp family selected from the group consisting of the hsp 70 family, the hsp90 family and the hsp60 family.

31. The kit of claim 23, wherein the hsp in the hsp-peptide complex is a member of an hsp family selected from the group consisting of the hsp 70 family, the hsp90 family and the hsp60 family.

32. The method of claim 1, 3, 5, 10, 12 or 13, wherein the hsp in the hsp-peptide complex is selected from the group consisting of hsp90, gp96 (grp94), hsp104, hsp 70 and hsp 60.

33. The kit of claim 23, wherein the hsp in the hsp-peptide complex is selected from the group consisting of hsp90, gp96 (grp94), hsp104, hsp 70 and hsp 60.

34. A purified complex comprising an oligomerized, immunoactive heat shock protein and an antigenic molecule; wherein the heat shock protein has been oligomerized by contact with an oligomerizing agent, with the proviso that the oligomerizing agent is not a lectin, 4,4′-dianilino-1,1′-binaphthyl-5,5′-disulfonic acid (“bis-ANS”), glutaraldehyde or sulfosuccinimidyl (4-azidosalicylamido) hexanoate (“SASD”).

35. A purified complex comprising an oligomerized, immunoactive heat shock protein and an antigenic molecule; wherein the heat shock protein has been oligomerized by covalent binding to an oligomerizing agent, with the proviso that the oligomerizing agent with the proviso that the oligomerizing agent is not bis-ANS, glutaraldehyde or SASD.

36. The complex of claim 34, wherein the heat shock protein is non-covalently bound to the oligomerizing agent.

37. The complex of claim 34 or 35, wherein the heat shock protein is gp96 or hsp90.

38. The complex of claim 34 or 35, wherein the heat shock protein and antigenic molecule are isolated as a complex from a cell lysate.

39. The complex of claim 26, wherein the cell lysate is from a cancerous cell or a cell infected with an agent displaying the antigenicity of an infectious disease agent.

40. A population of purified oligomerized complexes, each complex in said population comprising an immunoactive heat shock protein and an antigenic molecule, wherein the complex has been oligomerized by contact with an oligomerizing agent, with the proviso that the oligomerizing agent is not a lectin, bis-ANS, glutaraldehyde or SASD, and wherein at least one complex in said population comprises an antigenic molecule that is different from the antigenic molecule of another complex in said population.

41. A population of purified oligomerized complexes, each complex in said population comprising an immunoactive heat shock protein and an antigenic molecule, wherein the complex has been oligomerized by covalent binding to an oligomerizing agent, with the proviso that the oligomerizing agent is not bis-ANS, glutaraldehyde or SASD and wherein at least one complex in said population comprises an antigenic molecule that is different from the antigenic molecule of another complex in said population.

42. The population of complexes of claim 40 or 41, wherein the heat shock protein is gp96 or hsp90.

43. The population of complexes of claim 40 or 41, wherein the heat shock protein and antigenic molecule are isolated as a complex from a cell lysate.

44. The population of complexes of claim 43, wherein the cell lysate is from a cancerous cell or a cell infected with an agent displaying the antigenicity of an infectious disease agent.

45. A pharmaceutical composition comprising a pharmaceutically acceptable carrier and a purified complex, said complex comprising an oligomerized, immunoactive heat shock protein and an antigenic molecule, wherein the heat shock protein has been oligomerized by contact with an oligomerizing agent, with the proviso that the oligomerizing agent is not a lectin, bis-ANS, glutaraldehyde or SASD.

46. A pharmaceutical composition comprising a pharmaceutically acceptable carrier and a purified complex, said complex comprising an oligomerized, immunoactive heat shock protein and an antigenic molecule, wherein the heat shock protein has been oligomerized by covalent binding to an oligomerizing agent, with the proviso that the oligomerizing agent is not bis-ANS, glutaraldehyde or SASD.

47. A pharmaceutical composition comprising a pharmaceutically acceptable carrier and therapeutically effective dose of a purified complex, said complex comprising an oligomerized, immunoactive heat shock protein and an antigenic molecule, wherein the heat shock protein has been oligomerized by covalent binding to an oligomerizing agent.

48. The pharmaceutical composition of claim 45, 46 or 47, wherein the heat shock protein is gp96 or hsp90.

49. The pharmaceutical composition of claim 45, 46 or 47, wherein the complex is present in an amount effective for treatment or prevention of cancer or an infectious disease.

50. The pharmaceutical composition of claim 45, 46 or 47, wherein the heat shock protein and antigenic molecule are isolated as a complex from a cell lysate.

51. The pharmaceutical composition of claim 51, wherein the cell lysate is from a cancerous cell or a cell infected with an agent displaying the antigenicity of an infectious disease agent.

52. A kit comprising a purified oligomerized, immunoactive heat shock protein and an antigenic molecule; wherein the heat shock protein has been oligomerized by contact with an oligomerizing agent, with the proviso that the oligomerizing agent is not a lectin, bis-ANS, glutaraldehyde or SASD.

53. A kit comprising a purified oligomerized, immunoactive heat shock protein and an antigenic molecule; wherein the heat shock protein has been oligomerized by covalent binding to an oligomerizing agent, with the proviso that the oligomerizing agent is not bis-ANS, glutaraldehyde or SASD.

54. The kit of claim 52 or 53, wherein the heat shock protein is gp96 or hsp90.

55. The kit of claim 52 or 53, wherein the antigenic molecule displays the antigenicity of a cancer or of an agent of an infectious disease.

56. The kit of claim 55, wherein the heat shock protein and antigenic molecule are isolated as a complex from a cell lysate.

57. The kit of claim 56, wherein the cell lysate is from a cancerous cell or a cell infected with an agent displaying the antigenicity of an infectious disease agent.

58. A method of enhancing the antigenicity or immunogenicity of a complex comprising an immunoactive heat shock protein and an antigenic molecule by contacting the complex with an amount of an oligomerizing agent sufficient to cause oligomerization of the complex, with the proviso that the oligomerizing agent is not a lectin, bis-ANS, glutaraldehyde or SASD.

59. A method of enhancing the antigenicity or immunogenicity of a complex comprising an immunoactive heat shock protein and an antigenic molecule by contacting the complex with an amount of an oligomerizing agent sufficient to cause oligomerization of the complex; wherein the heat shock protein is covalently bound to the oligomerizing agent.

60. The method of claim 58 or 59, wherein the immunoactive heat shock protein is complexed with the antigenic molecule via a non-covalent bond.

61. The method of claim 58 or 59, wherein the antigenic molecule is a peptide.

62. The method of claim 58 or 59, wherein the complex comprising the immunoactive heat shock protein and the antigenic molecule is isolated from a cell lysate.

63. The method of claim 58 or 59, wherein the cell lysate is from a cancerous cell or a cell infected with an agent displaying the antigenicity of an infectious disease agent.

64. The method of claim 58 or 59, wherein the heat shock protein is gp96 or hsp90.

65. A method of treating or preventing a cancer or an infectious disease comprising administering to a subject in need of such treating or preventing, a therapeutically effective amount of a purified complex, wherein the complex comprises an oligomerized, immunoactive heat shock protein and an antigenic molecule; wherein the heat shock protein has been oligomerized by contact with an oligomerizing agent, with the proviso that the oligomerizing agent is not a lectin, bis-ANS, glutaraldehyde or SASD, and wherein the antigenic molecule displays the antigenicity of a tumor-specific or tumor-associated antigen of said cancer or of an agent of said infectious disease, respectively.

66. A method of treating or preventing a cancer or an infectious disease comprising administering to a subject in need of such treating or preventing, a therapeutically effective amount of a purified complex, wherein the complex comprises an oligomerized, immunoactive heat shock protein and an antigenic molecule; wherein the heat shock protein has been oligomerized by covalent binding to an oligomerizing agent, and wherein the antigenic molecule displays the antigenicity of a tumor-specific or tumor-associated antigen of said cancer or of an agent of said infectious disease, respectively.

67. The method of claim 65 or 66, wherein the heat shock protein is gp96 or hsp90.

68. The method of claim 65 or 66, wherein the complex comprising the immunoactive heat shock protein and the antigenic molecule is isolated from a cell lysate.

69. The method of claim 68, wherein the cell lysate is from a cancerous cell or a cell infected with an agent displaying the antigenicity of an infectious disease agent.

70. The method of claim 68, wherein the cell is obtained from said subject.

71. A method of treating or preventing a cancer or an infectious disease comprising administering to a subject in need of such treating or preventing, a therapeutically effective amount of a pharmaceutical composition comprising a pharmaceutically acceptable carrier and a purified complex, said complex comprising an oligomerized, immunoactive heat shock protein and an antigenic molecule, wherein the heat shock protein has been oligomerized by contact with an oligomerizing agent, with the proviso that the oligomerizing agent is not a lectin, bis-ANS, glutaraldehyde or SASD, and wherein the antigenic molecule displays the antigenicity of a tumor-specific or tumor-associated antigen of said cancer or of an agent of said infectious disease, respectively.

72. A method of treating or preventing a cancer or an infectious disease comprising administering to a subject in need of such treating or preventing, a therapeutically effective amount of a pharmaceutical composition comprising a pharmaceutically acceptable carrier and a purified complex, said complex comprising an oligomerized, immunoactive heat shock protein and an antigenic molecule, wherein the heat shock protein has been oligomerized by covalent binding to an oligomerizing agent, and wherein the antigenic molecule displays the antigenicity of a tumor-specific or tumor-associated antigen of said cancer or of an agent of said infectious disease, respectively.

73. The method of claim 71 or 72, wherein the heat shock protein is gp96 or hsp90.

74. The method of claim 71 or 72, wherein the complex comprising the immunoactive heat shock protein and the antigenic molecule is isolated from a cell lysate.

75. The method of claim 74, wherein the cell lysate is from a cancerous cell or a cell infected with an agent displaying the antigenicity of an infectious disease agent.

76. The method of claim 74, wherein the cell is obtained from said subject.

77. A method of making a pharmaceutical composition comprising:
(a) contacting a complex with an amount of an oligomerizing agent sufficient to cause oligomerization of the complex,
wherein the complex comprises an immunoactive heat shock protein and an antigenic molecule, with the proviso that the oligomerizing agent is not a lectin, bis-ANS, glutaraldehyde or SASD; and
(b) combining the oligomerized complex with a pharmaceutically acceptable carrier.

78. A method of making a pharmaceutical composition comprising:
(a) contacting a complex with an amount of an oligomerizing agent sufficient to cause oligomerization of the complex,
wherein the complex comprises an immunoactive heat shock protein and an antigenic molecule, and wherein the oligomerizing agent is covalently bound to the oligomerizing agent, with the proviso that the oligomerizing agent is not bis-ANS, glutaraldehyde or SASD; and
(b) combining the oligomerized complex with a pharmaceutically acceptable carrier.

79. The method of claim 77 or 78, wherein the heat shock protein and antigenic molecule are isolated as a complex from a cell lysate.

80. The method of claim 79, wherein the cell is a cancerous cell or a cell infected with an agent displaying the antigenicity of an infectious disease agent.

81. The method of claim 77 or 78, wherein the heat shock protein is gp96 or hsp90.
